SEQUENCE LISTING (22 sequences)

SEQ ID NO: 1 - DNA, 20 bp, synthetic construct
  source 1..20: mol_type = other DNA
atgccacattcctacgatta

SEQ ID NO: 2 - DNA, 19 bp, synthetic construct
  source 1..19: mol_type = other DNA
ttaaaacaggcggtttaaa

SEQ ID NO: 3 - DNA, 67 bp, synthetic construct
  source 1..67: mol_type = other DNA
cctctgttcccaaggggagaagccgaaacataaaaaggaggtcttttatgccacattcctacgatta

SEQ ID NO: 4 - DNA, 19 bp, synthetic construct
  source 1..19: mol_type = other DNA
ttaaaacaggcggtttaaa

SEQ ID NO: 5 - DNA, 40 bp, synthetic construct
  source 1..40: mol_type = other DNA
attaagaaggagatatacatatgaccaaggcaagtgtggc

SEQ ID NO: 6 - DNA, 41 bp, synthetic construct
  source 1..41: mol_type = other DNA
tctccccttgggaacagaggtcatgcctccacgctcaggag

SEQ ID NO: 7 - DNA, 39 bp, synthetic construct
  source 1..39: mol_type = other DNA
tcctgagcgtggaggcatgacctctgttcccaaggggag

SEQ ID NO: 8 - DNA, 41 bp, synthetic construct
  source 1..41: mol_type = other DNA
cgttgtacttttcggccttctcaaaaaagccggttcaggcc

SEQ ID NO: 9 - DNA, 42 bp, synthetic construct
  source 1..42: mol_type = other DNA
gcctgaaccggcttttttgagaaggccgaaaagtacaacgac

SEQ ID NO: 10 - DNA, 52 bp, synthetic construct
  source 1..52: mol_type = other DNA
gccacacttgccttggtcatatgtatatctccttcttaattaagcatgcgga

SEQ ID NO: 11 - DNA, 20 bp, synthetic construct
  source 1..20: mol_type = other DNA
tactccgggtacctgtgcgg

SEQ ID NO: 12 - DNA, 22 bp, synthetic construct
  source 1..22: mol_type = other DNA
ctacttgcagccgggcagaccc

SEQ ID NO: 13 - DNA, 22 bp, synthetic construct
  source 1..22: mol_type = other DNA
gaacgacggatgggagttctgg

SEQ ID NO: 14 - DNA, 27 bp, synthetic construct
  source 1..27: mol_type = other DNA
acatgtgagcaaaaggccagcaaaagg

SEQ ID NO: 15 - DNA, 15 bp, synthetic construct
  source 1..15: mol_type = other DNA
tgtgcgggctctaac

SEQ ID NO: 16 - DNA, 19 bp, synthetic construct
  source 1..19: mol_type = other DNA
ttaaaacaggcggtttaaa

SEQ ID NO: 17 - DNA, 38 bp, synthetic construct
  source 1..38: mol_type = other DNA
ctggccttttgctcacatgttactccgggtacctgtgc

SEQ ID NO: 18 - DNA, 35 bp, synthetic construct
  source 1..35: mol_type = other DNA
gacgtgttagagcccgcacactacttgcagccggg

SEQ ID NO: 19 - DNA, 35 bp, synthetic construct
  source 1..35: mol_type = other DNA
gtctgcccggctgcaagtagtgtgcgggctctaac

SEQ ID NO: 20 - DNA, 39 bp, synthetic construct
  source 1..39: mol_type = other DNA
agaactcccatccgtcgttcttaaaacaggcggtttaaa

SEQ ID NO: 21 - DNA, 39 bp, synthetic construct
  source 1..39: mol_type = other DNA
tttaaaccgcctgttttaagaacgacggatgggagttct

SEQ ID NO: 22 - DNA, 42 bp, synthetic construct
  source 1..42: mol_type = other DNA
ccgcacaggtacccggagtaacatgtgagcaaaaggccagca

(19) United States
(12) Patent Application Publication
(10) Pub. No.: US 2026/0117265 A1
(43) Pub. Date: Apr. 30, 2026

(54) ELECTRO-BIODIESEL EMPOWERED BY CO-DESIGN OF MICROORGANISM AND ELECTROCATALYSIS

(71) Applicant: THE TEXAS A&M UNIVERSITY SYSTEM, COLLEGE STATION, TX (US)

(72) Inventors: YUAN DAI, ST. LOUIS, MO (US); SHUHUA YUAN, ST. LOUIS, MO (US); KAINAN CHEN, ST. LOUIS, MO (US)

(21) Appl. No.: 19/375,922

(22) Filed: Oct. 31, 2025

Related U.S. Application Data
(60) Provisional application No. 63/714,207, filed on Oct. 31, 2024.

(51) Int. Cl. C12P 5/02; B01J 19/08; C10L 1/04; C12N 9/02; C12N 9/10; C25B 3/26; C25B 11/046

(52) U.S. Cl. CPC C12P 5/02; B01J 19/087; C10L 1/04; C12N 9/0008; C12N 9/0036; C12N 9/1029; C12N 9/104; C12Y 102/0101; C12Y 106/01001; C12Y 203/0102; C25B 3/26; C25B 11/046; C10L 2200/0476

(57) ABSTRACT

The subject invention pertains to environmentally-friendly compositions, methods and systems for the production of diesel biofuels. More specifically, the subject invention integrates electrocatalysis and microbial bioconversion to generate lipids from carbon dioxide in a way that increases energy efficiency over known biomass-derived diesel production and significantly reduces land use.

### CROSS-REFERENCE TO RELATED APPLICATION

This application claims the benefit of U.S. Provisional Application Ser. No. 63/714,207, filed Oct. 31, 2024, the disclosure of which is hereby incorporated by reference in its entirety, including all figures, tables and amino acid or nucleic acid sequences.

### STATEMENT REGARDING FEDERALLY SPONSORED RESEARCH

This invention was made with government support under NSF 2229160 and 2330245 awarded by the U.S. National Science Foundation. The government has certain rights in the invention.

### STATEMENT REGARDING SEQUENCE LISTING

The Sequence Listing for this application is labeled “Seq-List.xml” which was created on Oct. 29, 2025 and is 20,590 bytes. The entire content of the sequence listing is incorporated herein by reference in its entirety.

### BACKGROUND OF THE INVENTION

Efficient and sustainable energy production is essential for climate change mitigation, yet current approaches like biofuels or electro-fuels have limitations in efficiency and product profile. We advanced a new electro-biodiesel route via integrating electrocatalysis and bioconversion to produce lipids from CO₂ for biodiesel. We first revealed bioenergetic and metabolic limits in C2+ intermediate utilization through simulations and metabolomics, guiding the synthetic biology design to achieve reductant balance, more ATP production, efficient lipid conversion, and higher lipid yield. Furthermore, we discovered specific ratios of ethanol and acetate to achieve co-substrate synergy, empowering bimetallic catalyst design to improve bioconversion efficiency. The microbial and catalyst co-design achieved a solar-energy-to-molecule conversion efficiency of 4.5% for CO₂-to-lipid conversion. Electro-biodiesel leverages the high efficiency of electrocatalysis and longer-carbon-chain products from microbial lipid synthesis, overcoming the limitations for both electrocatalysis and bioconversion. Electro-biodiesel achieved 45-times-less land usage than soybean biodiesel, competitive economics, and substantial carbon emission reduction.

Long-range heavy-duty vehicles and industries such as mining, drilling, shipping, and construction heavily rely on energy-dense diesel fuels, which cannot be defossilized by electric vehicles. Biofuels can fill the gap, vet biodiesel productivity is limited by low photosynthesis efficiency. It is unrealistic to defossilize U.S. diesel consumption by a large percentage with biodiesel, considering the substantial land usage. A similar challenge exists to a broader scope, as the fundamental limit in human civilization lies in the efficient utilization of renewable energy to produce sufficient fuels, chemicals, materials, and food with limited natural resources. Herein, we developed a new electro-biodiesel concept to manufacture diesel from CO₂ by integrating electrocatalytic CO₂ reduction reaction (CO₂RR) to produce biocompatible C2+ intermediates with subsequent microbial conversion of these intermediates into lipids as biodiesel feedstock. The new concept can be broadly applied to the circular economy to manufacturing emission-negative fuels, chemicals, materials, and food ingredients at a much higher efficiency than photosynthesis and lower carbon emissions than petrochemicals. We have systemically addressed the challenges in electro-biomanufacturing by identifying the metabolic and biochemical limits of C2 utilization and overcame these limits by balancing the reducing equivalent and enhancing ATP production. Furthermore, we have revealed co-substrate effects for ethanol and acetate in engineered strains and co-designed copper-zinc bimetallic catalysts to produce an optimal ratio of acetate and ethanol to achieve higher bioconversion efficiency. The synergistic microbial and catalyst design empowered electro-biodiesel to achieve 4.5% solar-to-molecule efficiency for converting CO₂ to lipid, which is much more efficient than biodiesel and other competing platforms. Technoeconomic and life cycle analyses revealed competitive minimal selling prices, substantial carbon emissions, and dozens of times less land use compared to current biodiesels. The new concept can be broadly applied to chemical, material, and fuel manufacturing to create a circular carbon economy for mitigating global climate change. The electro-bioconversion of CO₂ thus could alleviate the biodiesel feedstock shortage and transform broad renewable fuel, chemical, and material manufacturing by defossilizing the sectors that are challenging to defossilize.

Most fuels are generated from sunlight by photosynthesis, either via directly converting plant products from CO₂ (e.g. soybean biodiesel and corn ethanol) or via processing the petroleum generated from photosynthesis millions of years ago (e.g. fossil fuels). The fundamental limits of human civilization lie in the energy-efficient CO₂ conversion into sufficient chemicals, fuels, materials, and food, to achieve fossil-fuel independence. In particular, it is critical to advance new routes to convert CO₂ into long-range heavy-duty vehicle fuels and aviation fuels, as both are challenging to defossilize with electric vehicles.¹ However, the current platforms all have their limitations.

Biodiesel has been explored as an alternative to fossil diesel. Biofuel production can effectively harness complex biological pathways to produce diverse chemicals for different types of fuels, including long-carbon-chain products like lipids for diesel (FIG. 1). However, the overall solar-to-chemical energy conversion efficiency of biofuels remains constrained by photosynthesis efficiency (FIG. 1).² Photosynthesis efficiency is typically limited to around 1% in terrestrial plants, which makes it impossible to fulfill the transportation needs with biofuels on limited arable lands.³ To fulfill the current U.S. diesel demand of 3.7 million barrels with soybean biodiesel, it will need 747 million acres of arable land, accounting for two times the U.S. arable land and 37% of the total land area of the continental 48 states of the U.S. (Table 2). While algal biodiesel has been proposed as an alternative with better land-use efficiency, algal biofuel faces commercialization challenges due to the inherent limitation on light penetration and harvesting cost.⁴ Energy-efficient pathways need to be developed to substantially surpass the photosynthesis energy conversion efficiency for the production of renewable diesel and aviation fuels.

Considering the biofuel limitations, electro-fuels and solar fuels have been proposed as alternative approaches. However, electrocatalysis and photocatalysis have inherent limits in producing longer carbon chain products, hindering the direct production of sustainable aviation and diesel fuels.^{5,6} Electrocatalysis has achieved high efficiency in CO₂RR (CO₂ Reduction Reactions) to produce C1 products like CO, formate, and methanol.⁷⁻⁹ Recent advancements have substantially improved Faraday efficiency for CO₂RR to C2 products like ethanol, acetate, and ethylene and even C4 products like butanol.¹⁰⁻¹⁴ However, achieving high catalyst selectivity, product yield, and titer for longer carbon chain products relevant to the diesel and aviation fuel series remains challenging.⁵

Here, we present an electro-biodiesel approach to efficiently produce biodiesel precursor, lipids, from CO₂, leveraging the integration of electrocatalysis and bioconversion and overcoming all aforementioned challenges (FIG. 1). Electro-biodiesel combines the high efficiency and conversion rate of electrocatalysis and the diverse product profile of microbial conversion.¹⁵ Early work has explored the possibility of integrating electrocatalytic CO₂RR with downstream bioconversion using C1 intermediates (formate, methanol, CO and such).¹⁶ However, the C1 intermediates (formate and methanol) and gas intermediates (CO and hydrogen) have limited microorganism selection for downstream bioconversion. It causes cell toxicity in many microbial species, needs multiple steps to enter primary metabolism, and often has gas-to-liquid transfer limitations, all of which translate to low conversion efficiency, rate, and titer. Recent breakthroughs leveraged advancements in electrocatalysis to generate C2+ intermediates for bioconversion, overcoming the challenges of inefficient intermediates for chem-bio interfacing and achieving the record productivity of bioplastics.¹⁵ Despite the progress, a substantial fundamental understanding of bioenergetic and metabolic limits is needed to achieve efficient conversion of CO₂ into broader products, including lipids. First, can C2 compounds fulfill the energy and carbon requirements for lipid biosynthesis? Second, what are the molecular and systems-level metabolic responses of microorganisms to these C2 compounds? Especially, are there any metabolic repressions and bioenergetic limits associated with their utilization? Third, is there a co-substrate effect of ethanol and acetate that enables more efficient lipid production? In this study, we conducted a comparative modeling simulation and systems biology analysis of a model oleaginous bacterium Rhodococcus jostii RHA1 to reveal the bioenergetic, metabolic, and biochemical limits for bioconversion of C2 intermediates to lipids. The fundamental understanding guided microbial design to overcome these limitations and leverage our previously discovered pivotal fatty acid synthase I (FASI) mechanism to achieve rapid lipid conversion. 17 Using the designed strain, we also discovered new co-substrate synergy, guiding the co-design of microorganisms and catalysts to achieve an even more efficient conversion of intermediates into lipids. This co-design approach enabled the electro-biodiesel system to achieve superior lipid productivity and energy conversion efficiency compared to prior studies.

### BRIEF SUMMARY OF THE INVENTION

The subject invention provides environmentally-friendly compositions, methods and systems for the improved production of diesel fuels. More specifically, the subject invention provides compositions, methods and systems for biodiesel feedstock production that integrate electrocatalysis and microbial bioconversion to generate long-chain lipids from carbon dioxide. Advantageously, in certain embodiments, the subject invention provides solutions that improve the environmental sustainability of producing and consuming biodiesel by, for example, increasing energy efficiency and productivity and reducing land use change, compared with production of other biomass-derived diesels.

In certain embodiments, the subject invention provides a composition for use in the production of diesel fuel lipid feedstock from CO₂, wherein the composition comprises an oleaginous microorganism and an electrocatalyst. The composition may further comprise a culture medium. The medium can, in some embodiments, comprise ethanol and/or acetate as the sole carbon source, in addition to sources of electrolytes and other medium components known in the art.

Preferably, the oleaginous microorganism is capable of expressing one or more lipid biosynthesis enzymes, either naturally or through genetic engineering, which contribute to enhanced ability to convert CO₂ into lipids. In certain embodiments, the microorganism is a Rhodococcus sp., for example, a strain of R. opacus and/or R. jostii. In a specific exemplary embodiment, the microbe is Rhodoccocus jostii RHA1.

In certain embodiments, the oleaginous microorganism expresses or overexpresses, either naturally or through genetic engineering, one or more of the following enzymes, which contribute to enhanced ability to convert CO₂ into lipids over wild-type strains: type 1 fatty acid synthase (FASI, encoded by fasI), diacylglycerol acyltransferase (DGAT, encoded by atf2), soluble pyridine nucleotide transhydrogenase (encoded by sthA) and/or acetaldehyde dehydrogenase (encoded by dmpF).

In one embodiment, the microorganism comprises, at least, fasI and atf2. In one embodiment, the microorganism comprises, at least, sthA and dmpF. In one embodiment, the microorganism's genome has been engineered to contain each of fasI, atf2, sthA and dmpF.

In certain embodiments, the composition comprises an electrocatalyst comprising low-microbe toxicity metallic species. Preferably, the electrocatalyst comprises Cu doped with Zn, and has the formula CuₓZn_{y}, where X and Y can each be any positive integer between 1-10. Even more preferably, the electrocatalyst is Cu₆Zn₁.

The subject invention further provides methods for producing a biodiesel feedstock wherein the methods comprise converting CO₂ into CO₂ intermediates using an electrocatalytic CO₂ reduction reaction (CO₂RR); and converting the CO₂ intermediates into lipids using an oleaginous microorganism as described herein. The method can then further comprise generating biodiesel from the lipids using known methods.

In preferred embodiments, the CO₂ intermediates contain two or more carbons and include, for example, ethanol and/or acetate. In further preferred embodiments, the method generates both ethanol and acetate at a ratio of 0.5 to 2.0, acetate/ethanol.

Advantageously, the methods of the subject invention can increase lipid productivity significantly over methods utilizing, for example, lipogenic algae.

Further provided herein is a system for continuous production of lipids from CO₂, which uses the compositions and methods of the subject invention to integrate electrocatalysis and bioconversion. In certain embodiments, the system comprises a two-chamber bioconversion unit (i.e., a bioreactor) and a CO₂ electrolysis unit (i.e., an electrolyzer) containing the bimetallic electrocatalyst.

To operate the system, the electrolyzer, and thus, the electrocatalyst, is connected to an electrical supply and a CO₂ supply. In certain preferred embodiments, electricity is supplied to the system using solar power.

Growth medium is circulated between the CO₂ electrolyzer and the first chamber of the bioconversion unit to accumulate CO₂ intermediates such as ethanol, acetate, propanol and formate, resulting from reduction reactions between the CO₂ and the electrocatalyst. The microbes are kept in the second chamber of the bioconversion unit, and separated from the first chamber using a non-selective filter membrane. The filter membrane allows the CO₂RR products to diffuse from the first chamber to the second chamber, while efficiently blocking microbes from entering the first chamber and the electrolyzer. The oleaginous microbe is thus provided access to the CO₂ intermediates, allowing for growth and production of lipids into the culture medium of the second chamber.

The subject invention provides a highly productive, efficient, cost-friendly, and reduced-carbon footprint electro-biodiesel production route that directly uses CO₂ as feedstock to fulfill industrial demands for diesel with significantly reduced land usage. Such land usage is not limited to arable lands, thus substantially alleviating food-energy competition and the shortage of biodiesel feedstock. Thus, the subject invention has the potential to revolutionize diesel-dependent sectors such as mining, drilling, shipping, and construction, which face substantial challenges in transitioning from fossil fuels.

### BRIEF DESCRIPTION OF THE DRAWINGS

The patent or application file contains at least one drawing executed in color. Copies of this patent or patent application publication, with color drawing(s), will be provided by the Office upon request and payment of the necessary fee.

FIG. 1. The schematic of routes to produce different types of diesel and fuels, with the comparisons in energy conversion efficiency, fuel carbon chain length, and land use. The arrows indicate the energy conversion direction. The values along the arrows represent the energy conversion efficiency (EE), which is referenced from literature. ^{†}The values are based on calculation by multiplying the efficiencies of each individual stage of the energy conversion processes. ^{§} The indicators refer to the carbon chain length of the primary fatty acids composing different fuels. ^{#} The values denote the land area required to sustain the annual total diesel consumption in the USA using these fuel types (Table 2). Illustration is created with BioRender.com.

FIG. 2. Computational simulation and experimental results of lipid fermentation using R. jostii RHA1 strain with ethanol or acetate as sole carbon source

(A) Stimulated FA biosynthesis fluxes of the R. jostii RHA1 GSM model under various carbon uptake rates for ethanol and acetate.

(B) Reactions that exhibit flux differences larger than 3 mmol/gDCW/h between ethanol and acetate in the FA biosynthesis simulation. The “rxn” stands for reaction, and the number in each well represents the magnitude of the flux of the reaction. The negative sign “-” indicates the reaction goes the reverse direction. A higher absolute value is masked by darker color, indicating a higher reaction flux. OAA: oxaloacetate; PEP: phosphoenolpyruvate; PP: pentose phosphate. The FBA analysis was conducted under the carbon uptake rate at 25 mmol/g DCW/h.

(C) Comparison of experimental and simulated FA accumulation of R. jostii RHA1 strain using ethanol or acetate as sole carbon source. C2 uptake: experimental C2 uptake rate (mg/gDCW/h)=C2 consumption (mg/L)/DCW titer (g/L)/time (h); Actual FA (mg/gDCW/h)=Lipid content (mg/gDCW)/time (h); Simulated FA: simulated FA accumulation rate (mg/gDCW/h) with experimental C2 uptakes, using the simulation curve in (A). Data used for the calculation can be found in FIG. 9. The legends are shared with D-F.

(D-H) Experimental lipid fermentation results using R. jostii RHA1 strain with ethanol or acetate as sole carbon source; (D) Cellular Acetyl-CoA level; (E) Cellular ATP level; (F) Cellular NADPH level. (G) Cellular NADH level; (H) The pH of culture after fermentation; RAU means relative absorbance units, RLU means relative luminescence units, and RFU means relative fluorescence units. All the data was collected with biological triplicates. All the values are presented in the form of mean±standard error of the mean. T-test was used to assess the significance of difference between the two groups. The * denotes a significant difference with p<0.05. ** for p<0.01, *** for p<0.001, and **** for p<0.0001.

FIG. 3. Visualization of the metabolic pathways based on differential primary metabolites and R. jostii RHA1 strains between the ethanol and acetate conditions. The metabolomic data was mapped onto Rhodococcus jostii RHA1 genome using the Pathway tools in Biocyc to get the metabolic pathways. The intermediates in the pathways are represented as circles, where gray circles specifically indicate phosphate intermediates. Arrows indicate the metabolic reactions; Dashed lines mean uncertain or simplified reactions. The reactions in green indicate potential metabolism involved in cellular acidification response, and the reactions in yellow indicate potential metabolism involved in combating reducing equivalent imbalance. GIP: glucose 6-phosphate; G6P: glucose 6-phosphate; F6P: fructose 6-phosphate; R5P: ribose 5-phosphate; Ru5P: ribulose 5-phosphate; S7P: sedoheptulose 7-phosphate; LG: levoglucosan; MI: myo-inositol; CBE: conduritol-beta-epoxide; EP: ethanol phosphate; BP: biphenyl; DEA: diethanolamine; EA: ethanolamine; PEA: phosphoethanolamine; C17FA: cis-10-heptadecenoic acid; C19FA: nonadecanoic acid; C21FA: behenic acid; Sph: D-erythro-sphingosine; G3P: glucose 3-phosphate; UDP-glu: uridine diphosphate glucose; UDP-GlcNAc: uridine diphosphate N-acetylglucosamine; 3PG: 3-Phosphoglycerate; DHAP: dihydroxyacetone phosphate; Gly3P: glycerol 3-phosphate; CDP-DAG: cytidine diphosphate diacylglycerol; TAG: triacylglycerol; Cer: ceramides; DMAPP: dimethylallyl pyrophosphate; Ser: serine; SerOP: Phosphoserine; Trp: tryptophan; Tyr: tyrosine; Phe: phenylalanine; Lys: lysine; Gln: glutamine; Glu: glutamate; Ala: alanine; Asp: aspartate; PP pathway: pentose phosphate pathway; FAS: fatty acid synthesis; TCA cycle: tricarboxylic acid cycle; VB5: pantothenic acid; MIA: 1-methyladenosine; CMP: cytidine-monophosphate; PsU: pseudouridine; Nam: nicotinamide; NAD: nicotinamide adenine dinucleotide; CTP: cytidine triphosphate; UMP: uridine monophosphate; MOB: 3-methyl-2-oxobutanoate; MTHF: 5,10)-methylenetetrahydrofolate. All metabolite levels were measured in biological triplicates and normalized for comparison. Fold change analysis and unpaired two-samples Wilcoxon test were used for analysis. T-test was used to assess the significance of difference between the two groups. The * denotes metabolites significantly different with p<0.05.

FIG. 4. Genetic engineering to enhance lipid biosynthesis from ethanol in R. jostil RHA1.

(A) Schematic of the lipid biosynthetic pathway from ethanol. Genes of fasI, atf2, dmpF, and sthA were overexpressed with the recombinant plasmid method. dmpF, a gene coding acetaldehyde dehydrogenase: sthA, coding soluble pyridine nucleotide transhydrogenase; fasI, coding type I fatty acid synthase; atf2, coding diacylglycerol acyltransferase. Arrows in color indicate the reactions that are upregulated by genetic manipulation. Illustration is created with BioRender.com.

(B-F) Experimental lipid fermentation results using the WT and engineering R. jostii RHA1 strains with ethanol as sole carbon source. The legends are shared. (B) Ethanol consumption; (C) Lipid content in DCW; (D) Cellular NADPH level; (E) Cellular ATP level; (F) Cellular NADH level. RAU means relative absorbance units, RFU means relative fluorescence units, RLU means relative luminescence units.

(G) The actual fatty acid (actual FA) accumulation rate based experimental data. Actual FA (mg/gDCW/h)=Lipid content (mg/gDCW)/time. WT:ethanol indicates WT strain using ethanol as sole carbon source; fads:ethanol indicates fads strain using ethanol as sole carbon source; WT:acetate indicates WT strain using acetate as sole carbon source; fads:acetate indicates fads strain using acetate as sole carbon source. Data used for the calculation can be found in FIG. 9 and FIGS. 5A and B.

Data was collected using biological triplicates, and all values are presented as mean±standard error of the mean. Turkey's multiple comparisons test was used to assess the significance of difference between the two groups. The * denotes a significant difference with p<0.05, ** for p<0.01, *** for p<0.001, and **** for p<0.0001. Only the significant difference in mean is shown in the figure.

FIG. 5. Bi-metallic design for optimal ratio of C2 substrates in RHA1 fads lipid production.

(A) The pH of the culture media.

(B-D) lipid contents (B), lipid titers (C), and C2 consumption (D) of the engineered fads strain when using ethanol, acetate, and mixed C2 as carbon sources with total concentration of 180 mmol/L. Ethanol means ethanol as used as the sole carbon source; Acetate/Ethanol=x denotes a mixed C2 carbon source with an x:1 mole ratio of acetate and ethanol; acetate indicates a sole acetate carbon source. The legends are shared with B, C, and D. The fads cells were prepared in LB broth to reach OD₆₀₀ at 1.0 and then transferred into the Rhodococcus media with the C2 as the sole carbon source for lipid production (see Methods).

(E) Bimetallic Cu/Zn catalysts for producing ethanol and acetate as main soluble C2 products.

(F) Schematic illustration of the customized three-chamber flow electrolyzer. The microbial cultural solution was used as the electrolyte solution to elute the generated soluble C2+ products.

(G) Faradaic efficiencies of soluble C2+ products (left y-axis, the bar chart with black confidence interval) and the acetate to ethanol ratios (right y-axis, blue dot chart with confidence interval) from Cu and Cu—Zn catalysts in cultural medium. The electroreduction was conducted under a total current of 500 mA, with electrode area 4 cm².

All data was collected using triplicates, and values are presented as mean #standard error of the mean. Turkey's multiple comparisons test was used to assess the significance of difference among the groups. The * denotes a significant difference with p<0.05. ** for p<0.01, *** for p<0.001. Only the significant difference in mean is shown in the figure.

FIG. 6. System integration, performance evaluation, energy efficiency estimation, and economic assessment.

(A) Schematic illustration of the integrated electro-biofuel system mainly consists of a CO₂ supplier, electrolyzer, pump, and bioreactor (Asymmetric dual-chamber with a 15 mL left chamber and a 45 mL right chamber). CO₂RR products include ethanol, acetate, propanol, and formate.

(B) The biocompatibility and stability of the integrated system demonstrated by the cell growth of WT RHA1 and the performance of the copper catalyst. The WT RHA1 was inoculated into the system to achieve rapid cell growth or biomass generation from CO₂. The blue curve in the upper box indicates the voltage of CO₂ electrocatalysis and the curves in the lower box indicate cell growth, concentrations of carbon substrates, and lipid titer during fermentation. The lipid contents in the inoculated and harvested RHA1 cells are 0.13 g/g DCW and 0.28 g/g DCW, respectively.

(C) Lipid production of different combinations of catalysts and strains in the integrated system. High cell density of RHA1 cells, with an OD₆₀₀ of approximately 4 to 5, is inoculated to serve as the whole cell catalyst for lipid production from CO₂. The Cu+WT represents a combination of copper-based catalyst and WT RHA1 strain. The Cu+fads represents a combination of copper-based catalyst and engineered strain fads. The Cu₆Zn₁+fads represents a combination of copper/zinc bimetallic catalyst and engineered strain fads. Cell density was indicated by dry cell weight and lipid production was calculated by deducting the initial lipid accumulation from the final lipid titer. Data was collected using biological triplicates, and all values are presented as mean±standard error of the mean.

(D) Comparison of lipid productivity of the electro-biofuel system with high lipid producing algae and microalgae studies. High lipid productivity cases were extracted from three recent reviews that focused on lipid-producing microalgae and algae to assess our electro-biofuel system. WT RHA1+Cu indicates the integrated system operates with wide-type RHA1 as lipid producer and copper catalyst for CO₂RR reduction; RHA1 fads+Cu indicates the integrated system operates with fads strain as lipid producer and copper catalyst for CO₂RR reduction; RHA1 fads+Cu₆Zn₁ indicates the integrated system operates with fads strain as lipid producer and Cu₆Zn₁ catalyst for CO₂RR reduction. Data on the lipid productivity of algae were reported in high lipid productivity cases that were obtained from three recent reviews focused on lipid-producing microalgae and algae.

(E) Calculation of overall energy efficiency of the electro-bio-fuel system. Data was collected using biological triplicates, and all values are presented as mean±standard error of the mean.

(F) Economic contribution of process stages to an upscaled electro-biodiesel system for lipid production at a scale of 8000 tons/year.

FIG. 7. Comparison of theoretical fatty acid yields from ethanol, acetate and glucose as sole carbo source. The previously reported calculation method, which is based on the stoichiometry of glycolysis, ethanol and acetate metabolism, tricarboxylic acid cycle and fatty acid synthesis (8 acetyl-CoA+7 ATP+14 NADPH→C16 palmitic acid), was adopted to calculate the yield.

FIG. 8. Comparison of cell growth among Rhodococcus species and strains. Each strain was inoculated in the minimal medium supplemented with 45 mmol/L ethanol or acetate as the sole carbon source per 12 hours to cultivate the growth. The OD₆₀₀ data was collected at 48 hours after inoculation using biological triplicates. All values are presented as mean±standard error of the mean.

FIG. 9. Experimental cell growth and lipid accumulation of R. jostii RHA1 strain with C2 substrates. The RHA1 strain was inoculated in the minimal medium with 225 mmol/L ethanol or acetate as sole carbon source in a 54-hour cell growth assay. (A) DCW production: (B) Lipid content in DCW: (C) C2 consumption. All the data was collected with biological triplicates. All the values are presented in the form of mean±standard error of the mean. T-test was used to assess the significance of difference between the two groups. The * denotes metabolites significantly different with p<0.05, ** for p<0.01, *** for p<0.001, and **** for p<0.0001.

FIG. 10. Visualization of the differential (t-test, p<0.05) lipid species of R. jostii RHA1 strains between different comparisons. Categorization and comparison of complex lipid species accumulated in R. jostii RHA1 in the conditions with ethanol or acetate as the sole carbon source. (A) comparison between ethanol and acetate conditions with WT (Wild-Type) strain (WT_ethanol vs WT_acetate); (B) comparison between WT and fads strains under ethanol condition (WT_ethanol vs fads_ethanol); (C) comparison between WT and fads strains under acetate condition (WT_acetate vs fads_acetate); (D) comparison between ethanol and acetate conditions with fads strain (fads_ethanol vs fads_acetate). WT indicates the wild type RHA1 strain. The fads represents fasI-atf2-dmpF-sthA strain in which genes of dmpF, sthA, fasI, and atf2 were overexpressed. On the left side of each plot, the lipid category is indicated. The color of the bars represents different strains or conditions, and the number within each bar indicates the counts of the significantly higher lipids under that condition. TAG: triacylglycerols: FA: Fatty Acids and Conjugates: GPE: Glycerophosphoethanolamines: GPI: Glycerophosphoinositols: GroG: Glycerophosphoglycerols; DAG: Diacylglycerols; GroGroG: Glycerophosphoglycerophosphoglycerols; FAcyl: Fatty Acyls; FAE: Fatty esters; GPCho: Glycerophosphocholines; Cer: Ceramides; GSL: Glycosphingolipids; GPL: Glycerophospholipids; GL: Glycerolipids. All the data was collected with biological triplicates.

FIG. 11. Categorization of the 70 primary metabolites showing significantly higher levels in the WT RHA1 cells on ethanol substrate compared to acetate substrate.

FIG. 12. The impact of metabolomics-identified metabolites on cell growth and pH of WT RHA1 culture.

(A) Cell growth of RHA1 on 45 mmol/L/12 h ethanol as carbon source with supplementation of different metabolites with a rate of 0.1 g/L/12 h. (B) The pH of culture media after 48-hour culturing.

FIG. 13. Cell growth, lipid production, and physiological assay of RHA1 strains on ethanol substrate with and without pH adjustment.

(A) Lipid and biomass in total DCW (Dry Cell Weight); (B) Cellular ATP level; (C) C2 consumption; (D) Cellular NAD(P)H level. Data was collected with biological triplicates, and all values are presented as mean±standard error of the mean. T-test was used to assess the significance of difference between the two groups. The * denotes metabolites significantly different with p<0.05, ** for p<0.01, *** for p<0.001, and **** for p<0.0001.

FIG. 14. The pH changes of the culture media (A) and the acetate ambulation-to-ethanol consumption ratio of WT and engineered RHA1 strains (B) when using ethanol as the sole carbon source.

WT indicates the wild type RHA1 strain. The dmpF-sthA indicates the RHA1 strain with gene overexpression of dmpF and sthA. The fasI-atf2 indicates the RHA1 strain with gene overexpression of fasI and atf2. The dmpF-sthA-fasI-atf2 indicates the RHA1 strain with gene overexpression of dmpF, sthA, fasI, and atf2. Data was collected using biological triplicates, and all values are presented as mean±standard error of the mean. Turkey's multiple comparisons test was used to assess the significance of difference between the two groups. The * denotes metabolites significantly different with p<0.05, ** for p<0.01, *** for p<0.001, and **** for p<0.0001. Only the significant difference in mean is shown in the figure.

FIG. 15. Experimental lipid fermentation results using the WT and fasI-atf2 strains with acetate as the sole carbon source.

(A) Lipid content in DCW: (B) Lipid titer: (C) Cellular ATP level; (D) Cellular NADPH level. (E) Cellular NADH level. RAU means relative absorbance units, RLU means relative luminescence units, and RFU means relative fluorescence units. Data was collected with biological triplicates, and all values are presented as mean±standard error of the mean. T-test was used to assess the statistical significance of the difference between the two groups. The * denotes metabolites significantly different with p<0.05, ** for p<0.01. *** for p<0.001, and **** for p<0.0001.

FIG. 16. Visualization of the differential (Abslog 2 (FC)>1) primary metabolites of RHA1 strains between the ethanol and acetate conditions. The involved metabolic pathways based on the differential metabolite profile of the R. jostii RHA1 strains between conditions with ethanol or acetate as sole carbon source. The order of the box in the legend bar stands for 4 groups: (1) WT strain in ethanol (WT_ethanol): (2) WT strain in acetate (WT_acetate): (3) fads strain in ethanol (fads_ethanol); and (4) fads strain in acetate (fads_acetate). The color of each box indicates the metabolite's relative level according to the scale in the upright conner. The intermediates in the pathways are represented as circles, where gray circles specifically indicate phosphate intermediates. Carbohydrate metabolisms were highlighted in yellow color, amino acids biosynthesis pathways were highlighted in pink color, tRNA charging reactions were highlighted in green color, metabolism related with nucleosides were highlighted in blue color. Glc-6P: galactose 6-phosphate: ADP-glu: ADP-a-D-glucose: Glc-IP: galactose 1-phosphate; GlgE-Gly: GlgE-Glycogen; UDP-GlcNAc: UDP-N-acetyl-α-D-glucosamine; GlcNAc: N-acetyl-α-D-glucosamine: G6P: D-glucopyranose 6-phosphate: PPP: pentose phosphate pathway; Ru5P: D-ribulose 5-phosphate: F6P: β-D-fructose-6-phosphate; UDP-Glu: UDP-α-D-glucose; NAD: Nicotinamide adenine dinucleotide; CMP: cytidine-monophosphate; D-threo-ict: D-threo isocitrate; asp: aspartate; lys: lysine; thr: threonine; ile: isoleucine; met: methionine; asn: asparagine; ala: alanine; ser: serine; L-hse: L-homoserine; D-ala: D-alanine; phe: phenylalanine; val: valine; arg: arginine; glu: glutamate; gln: glutamine; MOB: 3-methyl-2-oxobutanoate: 10-CH2-THF: 5,10-methylenetetrahydrofolate; PyrN: pyrimidine ribonucleotides; PRPP: 5-phospho-α-D-ribose 1-diphosphate; CTP: cytidine triphosphate; IMP: inosine-5′-phosphate; AIR: 5-amino-1-(5-phospho-β-D-ribosyl) imidazole; dATP: deoxyadenosine triphosphate; CoA: Coenzyme A; tRNA: transfer ribonucleic acid; sp: superpathway. All the metabolite levels were measured in biological triplicates and normalized for comparison. The tool Pathway Collages in website METACYC was used to visualize the pathways.

FIG. 17. Co-substrate effect of different ratios of ethanol and acetate on lipid production (A) and C2 consumption (B) of WT RHA1. Ethanol: Ethanol as the sole carbon source: Acetate: Acetate as the sole carbon source: Mix: Mixed carbon source of ethanol and acetate. The carbon ratios in the mixed conditions are indicated alongside each annotation. For example, Mix_4:1: Mole ratio of ethanol to acetate is 4:1 in the mixed carbon source. Rhodococcus media with C2 as sole carbon source was used to grow the RHA1 strain from an initial OD₆₀₀ at about 0.2 for 54 hours. All data were collected with biological triplicates, analyzed with GraphPad Prism 9.0.0 and presented as mean±standard error of mean.

FIG. 18. X-ray photoelectron spectroscopy (XPS) analysis for Cu₆Zn₁ catalyst. (A) survey, (B) Cu 20, and (C) Zn 2p.

FIG. 19. SEM and EDS elemental mapping of Cu₆Zn₁ catalyst. (A and D) Low-magnification SEM image of the Cu and Cu₆Zn₁ catalyst. (B and E) Overall scan of EDS mapping. (C, F, and G) Images by Cu or Zn elements. Bottom: table of measured relative composition of Cu and Zn elements of Cu₆Zn₁ catalyst.

FIG. 20. Faradic efficiency of C2+ oxygenates on catalysts with various Cu/Zn atomic ratios at different current densities.

FIG. 21. Faradic efficiency of generated products over the catalysts with different Zn loading amounts.

FIG. 22. CO₂RR product profile and stability test of Cu₆Zn₁ catalyst. (A) Faradic efficiency of CO₂RR products of Cu₆Zn₁ at various total current densities. Partial current densities of each product, as J_{product}=Jₜₒₜₐₗ×FE_{product}. (B) The ¹H NMR spectra for the liquid products after the reaction. (C) 72-hour stability test with the phosphate buffer used as both the electrolyte and the Rhodococcus media.

FIG. 23. Electro-microbial integration setup (A) and the two-electrode electrolyzer configuration (B).

FIG. 24. Effect of formate and propanol as carbon sources on cell growth of WT RHA1. (A) Cell growth of RHA1 when using each component of soluble CO₂RR products as sole carbon source. (B) Substrate consumption of RHA1 when growing on each component of soluble CO₂RR products. (C) Cell growth of RHA1 when C2 substrate supplementing with formate or propanol. (D) Substrate consumption of RHA1 when growing on C2 substrate supplementing with formate or propanol. Each substrate is provided at a 45 mmol/L per 12 hours.

FIG. 25. Effect of formate and propanol as carbon source on lipid production of WT RHA1. (A) Lipid content of RHA1 when with C2 supplementing with formate or propanol. (B) Substrate consumption of RHA1 when growing on each component of soluble CO₂RR products. The C2 indicate a mixture of ethanol and acetate, which were supplemented with 60 mmol/L/12 hour and 30 mmol/L/12 hour, respectively. “F” indicates formate, and “P” indicates propanol. They are supplemented at 50 mmol/L and 2.2 mmol/L every 12 hours, based on their ratios in the CO₂RR product profile.

FIG. 26. The boundary of the electro-biodiesel system in LCA analysis.

FIG. 27. Scenario analysis for CO₂RR-Lipid system. Scenarios 1 and 2 assumed the system used electricity from the U.S. Natural gas combined cycle (NGCC) power plants with Carbon Capture and Storage (CCS) equipment. Scenarios 3 and 4 assume the system is powered by renewable electricity without extra GHG emissions. Scenarios 2 and 4 made an additional assumption that the byproducts from the system can displace conventional products and offset the GHG emissions from conventional sources.

FIG. 28. Sensitivity analysis for the minimum selling price of lipids ($/kg) based on the electro-biodiesel system. The baseline scenario (vertical line) was calculated using the second number in each label as the input data, which results in a minimal selling price of lipids at $2.36/kg. The optimistic and pessimistic scenario analysis was then made assuming the current analyzed parameter taking the optimistic assumption (the first number in the label) or the pessimistic assumption (the third number in the label), but all other parameters taking the baseline assumptions (the second number in the labels).

FIG. 29. Relationship of OD₆₀₀ and DCW of R. jostii RHA1 strain.

### DETAILED DISCLOSURE OF THE INVENTION

The subject invention provides environmentally-friendly compositions, methods and systems for the improved production of diesel biofuel feedstock.

As used herein, each of the following terms have the meanings associated with it as specified below. Unless defined otherwise, all technical and scientific terms used herein generally have the same meaning as commonly understood by one of ordinary skill in the art to which this invention belongs.

The term “biofuel” is a fuel source produced from renewable organic material. The organic material is typically referred to as “feedstock.”

The term “alteration” refers to a change (increase or decrease) in the expression levels or activity of a gene or polypeptide as detected by standard art known methods such as those described herein. As used herein, an alteration includes at least a 10% change in expression levels, preferably at least a 25% change, more preferably at least a 40% change, and most preferably at least a 50% or greater change in expression levels.

The term “host cell” refers to a cell into which a nucleic acid of interest is to be transformed.

The term “transformation” refers to a permanent or transient genetic change, preferably a permanent genetic change, induced in a cell following incorporation of non-host nucleic acid sequences. “Transformed” can be used interchangeably with “engineered” herein.

The term “vector” generally refers to a polynucleotide that can be propagated and/or transferred between organisms, cells, or cellular components. Vectors include viruses, bacteriophage, pro-viruses, plasmids, phagemids, transposons, and artificial chromosomes, that are able to replicate autonomously or can integrate into a chromosome of a host cell. A vector can also be a naked RNA polynucleotide, a naked DNA polynucleotide, a polynucleotide composed of both DNA and RNA within the same strand, a poly-lysine-conjugated DNA or RNA, a peptide-conjugated DNA or RNA, a liposome-conjugated DNA, or the like, that are not episomal in nature, or it can be an organism which comprises one or more of the above polynucleotide constructs such as an agrobacterium.

The term “promoter” refers to a minimal nucleic acid sequence sufficient to direct transcription of a nucleic acid sequence to which it is operably linked. The term “promoter” is also meant to encompass those promoter elements sufficient for promoter-dependent gene expression controllable for cell-type specific expression or inducible by external signals or agents: such elements may be located in the 5′ or 3′ regions of the naturally-occurring gene. In some embodiments, the engineered microorganisms disclosed herein comprise fasI, atf2, sthA and/or dmpF genes that are under the control of a constitutive promoter. The constitutive promoter can be used to control each gene individually or any combination of fasI, atf2, sthA and/or dmpF genes.

An engineered or modified microorganism can also include in the alternative or in addition to the introduction of a genetic material into a host or parental microorganism, the disruption, deletion, or knocking out of a gene or polynucleotide to alter the cellular physiology and biochemistry of the microorganism (e.g., disruption, deletion, or knocking out of fasI, atf2, sthA and/or dmpF genes that naturally occur in the genetic material of a host or parental microorganism). Through the reduction, disruption or knocking out of a gene or polynucleotide the microorganism acquires new or improved properties (e.g., the ability to produce a new or greater quantities of an intracellular metabolite, improve the flux of a metabolite down a desired pathway, and/or reduce the production of undesirable by-products).

By “fragment” is meant a portion of a polypeptide or nucleic acid molecule. This portion contains, preferably, at least 10%, 20%, 30%, 40%, 50%, 60%, 70%, 80%, 90%, 95%, or 99% of the entire length of the reference nucleic acid molecule or polypeptide. A fragment may contain 10, 20, 30, 40, 50, 60, 70, 80, 90, 100, 200, 300, 400, 500, 600, 700, 800, 900, or 1000 nucleotides or amino acids, or more.

By “gene” is meant a locus (or region) of DNA that encodes a functional RNA or protein product.

By “overexpression” of a gene is meant expression of the gene at higher levels than normal, resulting in a greater production of its corresponding protein. The increase in expression can be at least a 0.25%, 0.5%, 1%, 5%, 10%, 15%, 20%, 25%, 30%, 35%, 40%, 45%, 50%, 55%, 60%, 65%, 70%, 75%, 80%, 85%, 90%, 95% or 100% increase in the expression of a given gene relative to the normal expression of the gene in a reference organism (e.g., overexpression of a gene in a genetically modified microorganism is compared to the expression of the same gene in a reference microorganism that does not contain the genetic modification and both the genetically modified microorganism and reference microorganism are of the same genus, species, and, optionally, the same strain). In some embodiments, overexpression of a gene can be caused by the use of promoters, such as constitutive promoters controlling a gene (e.g., fasI, atf2, sthA and/or dmpF genes). In other embodiments, overexpression of the gene can be caused by engineering the microorganism to contain and express multiple copies of a gene (e.g., fasI, atf2, sthA and/or dmpF genes) under the control of a native promoter (e.g., a native constitutive promoter) or a heterologous promoter (e.g., a heterologous constitutive promoter).

By “modulate” is meant alter (increase or decrease). Such alterations are detected by standard art known methods such as those described herein.

Nucleic acids include but are not limited to: deoxyribonucleic acid (DNA), ribonucleic acid (RNA), double-stranded DNA (dsDNA), single-stranded DNA (ssDNA), messenger RNA (mRNA), ribosomal RNA (rRNA), transfer RNA (tRNA), micro RNA (miRNA), and small interfering RNA (siRNA).

By “reference” is meant a standard or control condition.

A “reference sequence” is a defined sequence used as a basis for sequence comparison or a gene expression comparison. A reference sequence may be a subset of or the entirety of a specified sequence: for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 40 nucleotides, preferably at least about 60 nucleotides, more preferably at least about 75 nucleotides, and even more preferably about 100 nucleotides or about 300 or about 500 nucleotides or any integer thereabout or there between.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% or more identical at the amino acid level or nucleic acid level to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine: valine, isoleucine, leucine: aspartic acid, glutamic acid, asparagine, glutamine: serine, threonine: lysine, arginine; and phenylalanine, tyrosine. In an exemplary approach to determining the degree of identity, a BLAST program may be used, with a probability score between e-3 and e-100 indicating a closely related sequence.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

As used herein, “reduction” means a negative alteration and “increase” means a positive alteration, wherein the positive or negative alteration is at least 0.25%, 0.5%, 1%, 5%, 10%, 15%, 20%, 25%, 30%, 35%, 40%, 45%, 50%, 55%, 60%, 65%, 70%, 75%, 80%, 85%, 90%, 95% or 100%.

The transitional term “comprising,” which is synonymous with “including,” or “containing,” is inclusive or open-ended and does not exclude additional, un-recited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention. Use of the term “comprising” contemplates other embodiments that “consist” or “consist essentially of” the recited component(s).

Unless specifically stated or obvious from context, as used herein, the term “or” is understood to be inclusive. Unless specifically stated or obvious from context, as used herein, the terms “a,” “and” and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”. The transitional terms/phrases (and any grammatical variations thereof) “comprising”, “comprises”, “comprise”, “consisting essentially of”, “consists essentially of”, “consisting” and “consists” can be used interchangeably.

The term “and/or” as used herein refers to and encompasses any and all possible combinations of one or more of the associated listed items. For example, the phrase “A, B, and/or C” includes A alone, B alone, C alone, the combination of A and B, the combination of A and C, the combination of B and C, and the combination of A, B, and C. Also, the term “or” is used in its inclusive sense (and not in its exclusive sense) so that when used, for example, to connect a list of items, the term “or” means one, some, or all of the items in the list. Conjunctive language such as the phrase “at least one of X, Y, and Z,” unless specifically stated otherwise, is otherwise understood with the context as used in general to convey that an item, term, etc. may be either X, Y, or Z: X and Y: X and Z: Y and Z: or X, Y, and Z (i.e., any combination of X, Y, and Z).

All references cited herein, including patents, patent applications, and non-patent literature, are hereby incorporated by reference in their entirety.

### Compositions

In certain embodiments, the subject invention provides a composition for use in the production of diesel fuel lipid feedstock from CO₂, wherein the composition comprises an oleaginous microorganism and an electrocatalyst.

In some embodiments, the composition may further comprise a culture medium. In one embodiment, the culture medium comprises one or more sources of carbon. The carbon source can be a carbohydrate, such as glucose, dextrose, sucrose, lactose, fructose, trehalose, mannose, mannitol, and/or maltose: organic acids such as acetic acid, fumaric acid, citric acid, propionic acid, malic acid, malonic acid, and/or pyruvic acid; alcohols such as ethanol, propanol, butanol, pentanol, hexanol, isobutanol, and/or glycerol: fats and oils such as canola oil, madhuca oil, soybean oil, rice bran oil, olive oil, corn oil, sunflower oil, sesame oil, and/or linseed oil: powdered molasses, etc. These carbon sources may be used independently or in a combination of two or more. In certain embodiments the carbon sources are acetate, ethanol, propanol and/or formate.

In one embodiment, the culture medium comprises a nitrogen source. The nitrogen source can be, for example, yeast extract, potassium nitrate, ammonium nitrate, ammonium sulfate, ammonium phosphate, ammonia, urea, and/or ammonium chloride. These nitrogen sources may be used independently or in a combination of two or more.

In one embodiment, one or more inorganic salts may also be included in the culture medium. Inorganic salts can include, for example, potassium dihydrogen phosphate, monopotassium phosphate, dipotassium hydrogen phosphate, disodium hydrogen phosphate, potassium chloride, magnesium sulfate, magnesium chloride, iron sulfate, iron chloride, manganese sulfate, manganese chloride, zinc sulfate, lead chloride, copper sulfate, calcium chloride, calcium carbonate, calcium nitrate, magnesium sulfate, sodium phosphate, sodium chloride, and/or sodium carbonate. These inorganic salts may be used independently or in a combination of two or more.

In one embodiment, growth factors and trace nutrients for microorganisms are included in the culture medium. Inorganic nutrients, including trace elements such as iron, zinc, copper, manganese, molybdenum and/or cobalt may also be included in the medium. Furthermore, sources of vitamins, essential amino acids, proteins and microelements can be included, for example, corn flour, peptone, yeast extract, potato extract, beef extract, soybean extract, banana peel extract, and the like, or in purified forms. Amino acids such as, for example, those useful for biosynthesis of proteins, can also be included. Other components suitable for the growth of microorganisms are known in the art and can be readily ascertained by the skilled artisan.

The subject disclosure provides genetically modified microorganisms having advantageous properties related to the conversion of CO₂ to lipids. In some embodiments, the microorganism can be utilized at a cell density of about 0.5 to 10 g/L, or about 1 to 8 g/L, or about 2 to 7 g/L, or about 3 to 6 g/L, or about 4 to 5 g/L.

In one embodiment, the subject invention pertains to the genetic transformation of host cells (e.g., Gram positive or Gram negative bacteria) so as to provide these bacteria with enhanced ability to convert CO₂ to lipids. Thus, the subject invention allows the use of recombinant strains of Gram positive and/or Gram negative bacteria for the conversion of CO₂ to lipids and/or the use of these engineered strains as described herein.

Preferably, the oleaginous microorganism of the subject invention is capable of expressing one or more lipid biosynthesis enzymes, either naturally or through genetic engineering, which contribute to enhanced ability to convert CO₂ into lipids.

The host cell may be, selected from, for example, Gluconobacter oxydans, Gluconobacter asaii, Achromobacter delmarvae, Achromobacter viscosus, Achromobacter lacticum, Agrobacterium tumefaciens, Agrobacterium radiobacter, Alcaligenes faecalis, Arthrobacter citreus, Arthrobacter tumescens, Arthrobacter paraffineus, Arthrobacter hydrocarboglutamicus, Arthrobacter oxydans, Aureobacterium saperdae, Azotobacter indicus, Brevibacterium ammoniagenes, divaricatum, Brevibacterium lactofermentum, Brevibacterium flavum, Brevibacterium globosum, Brevibacterium fuscum, Brevibacterium ketoglutamicum, Brevibacterium helcolum, Brevibacterium pusillum, Brevibacterium testaceum, Brevibacterium roseum, Brevibacterium immariophilium, Brevibacterium linens, Brevibacterium protopharmiae, Corynebacterium acetophilum, Corynebacterium glutamicum, Corynebacterium callunae, Corynebacterium acetoacidophilum, Corynebacterium acetoglutamicum, Enterobacter aerogenes, Erwinia amylovora, Erwinia carotovora, Erwinia herbicola, Erwinia chrysanthemi, Flavobacterium peregrinum, Flavobacterium fucatum, Flavobacterium aurantinum, Flavobacterium rhenanum, Flavobacterium sewanense, Flavobacterium breve, Flavobacterium meningosepticum, Micrococcus sp. CCM825, Morganella morganii, Nocardia opaca, Nocardia rugosa, Planococcus eucinatus, Proteus rettgeri, Propionibacterium shermanii, Pseudomonas synxantha, Pseudomonas azotoformans, Pseudomonas fluorescens, Pseudomonas ovalis, Pseudomonas stutzeri, Pseudomonas acidovolans, Pseudomonas mucidolens, Pseudomonas testosteroni, Pseudomonas aeruginosa, Rhodococcus erythropolis, Rhodococcus opacus, Rhodcoccus jostii, Rhodococcus rhodochrous, Rhodococcus sp. ATCC 15592, Rhodococcus sp. ATCC 19070, Sporosarcina ureae, Staphylococcus aureus, Vibrio metschnikovii, Vibrio tyrogenes, Actinomadura madurae, Actinomyces violaceochromogenes, Kitasatosporia parulosa, Streptomyces coelicolor, Streptomyces flavelus, Streptomyces griseolus, Streptomyces lividans, Streptomyces olivaceus, Streptomyces tanashiensis, Streptomyces virginiae, Streptomyces antibioticus, Streptomyces cacaoi, Streptomyces lavendulae, Streptomyces viridochromogenes, Aeromonas salmonicida, Bacillus pumilus, Bacillus circulans, Bacillus thiaminolyticus, Bacillus coagulans, Escherichia freundii, Microbacterium ammoniaphilum, Serratia marcescens, Salmonella typhimurium, Salmonella schottmulleri, Xanthomonas citri, Thermotoga martima, Geobacillus sterothermophilus and so forth.

In certain embodiments, the host cell is a Rhodococcus sp., for example, a strain of R. opacus and/or R. jostii.

In certain embodiments, the oleaginous microorganism expresses or overexpresses, either naturally or through genetic engineering, one or more enzymes that perform any of the following functions as part of a bioconversion process: condensation and elongation of fatty acids; biosynthesis of triacylglycerols (TAGs); increase of CO₂ intermediate uptake by the expressing microorganism; decrease of acetic acid accumulation in culture medium; increase of NADPH supply; conversion of acetaldehyde into acetyl-CoA; mitigation of acetaldehyde-to-acetic acid oxidation; increase of ATP levels; supplying of NADH for NADPH production; and mitigation of medium acidification.

In certain embodiments, the oleaginous microorganism expresses or overexpresses, either naturally or through genetic engineering, one or more of the following enzymes, which contribute to enhanced ability to convert CO₂ into lipids over wild-type strains:

TABLE 1
Enzymes for Enhanced Lipid Biosynthesis from C2 Intermediates

| Name | Gene/operon | Source |
|---|---|---|
| FASI (type 1 fatty acid | fasI | BRENDA:EC 2.3.1.85 |
| synthase) | | GenBank Accession No. EHI42729.1 |
| DGAT (diacylglycerol | atf2 | BRENDA:EC 2.3.1.20 |
| acyltransferase) | | See also Hernandez et al., (2013) Appl |
| | | Microbiol Biotechnol. 97: 2119-2130. |
| soluble pyridine nucleotide | sthA | BRENDA:EC 1.6.1.1 |
| transhydrogenase | | |
| acetaldehyde dehydrogenase | dmpF | BRENDA:EC 1.2.1.10 |
| | | See also DmpF protein in Pseudomonas sp |
| | | CF₆₀₀ (accession no: CAA43226) (Shingler et |
| | | al. (1992) J. Bacteriol. 174: 71 1-24), and its |
| | | homologous MphF protein in E. coli (accession |
| | | no: NP_414885) (Ferrandez et al. (1997) J. |
| | | Bacteriol. 179: 2573-2581). |

All citations to patent- and non-patent literature, including the references and accession numbers mentioned above, are incorporated herein by reference in their entirety, including any nucleic acid and/or amino acid sequences described therein.

In one aspect of the subject invention Gram negative and/or Gram positive organisms are engineered to express or overexpress, at least, fasI and atf2. Advantageously, this combination of enzymes can, for example, significantly improve ethanol uptake by the microorganism, decrease acetic acid accumulation in the culture medium, and/or improve overall lipid accumulation.

In another aspect of the subject invention Gram negative and/or Gram positive organisms are engineered to express or overexpress, at least, sthA and dmpF. Advantageously, this combination of enzymes can, for example, increase NADPH supply and ATP levels to boost lipid production, and/or mitigate the acidification of the culture medium, thus reducing the oxidation of ethanol into acetic acid.

In one embodiment, the microorganism has been transformed to express and/or overexpress each of fasI, atf2, sthA and dmpF. In certain embodiments, each of these genes is overexpressed, optionally under the control of a constitutive promoter. The promoter may be native or heterologous. In certain embodiments, the promoter is a heterologous constitutive promoter.

The host microorganisms that are transformed may, or may not, contain a naturally occurring fasI, atf2, sthA and/or dmpF. In some embodiments, the transformed organism lacks a naturally occurring fasI, atf2, sthA and/or dmpF gene. In other embodiments, the Gram negative and/or Gram positive organisms are engineered such that endogenous or naturally occurring fasI, atf2, sthA and/or dmpF genes are inactivated or not expressed (e.g., the genes are deleted or contain insertions such that enzymatic activity of the gene products is reduced or eliminated).

In a specific exemplary embodiment, the subject invention provides a strain of Rhodoccocus jostii RHA1 that been engineered to contain each of fasI, atf2, sthA and dmpF. In one embodiment, each of these genes is overexpressed. This microbe is particularly well-suited for maximizing lipid production when utilized in combination with the electrocatalyst according to the subject invention.

To impart to a microorganism the ability to produce one or more of the enzymes disclosed herein, a single nucleic acid comprising any or all of the encoding genes can be provided to a bacterial cell via transformation or any other means (e.g., chromosomal integration). These elements may be used for the direct production of the desired enzymes. Alternatively, individual nucleic acids (e.g., genes) can be used to transform the host cell. Thus, a single nucleic acid molecule according to the subject invention can contain one or any combination of fasI, atf2, sthA and dmpF genes. The individual nucleic acids can be incorporated into a plasmid or other genetic construct that is used to transform a host organism and controlled by a native or heterologous promoter. In certain embodiments, the promoter is a constitutive promoter or an inducible promoter. In some embodiments, control of one or any combination of the fasI, atf2, sthA and dmpF genes is under the control of a constitutive promoter. Constitutive and/or inducible promoters suitable for use within the context of this disclosure are known to those skilled in the art.

Also within the scope of the subject instant invention are vectors or expression cassettes containing genetic constructs as set forth herein or polynucleotides encoding the enzymes, set forth supra, operably linked to regulatory elements, such as native or heterologous promoters (e.g., inducible or constitutive promoters). The vectors and expression cassettes may contain additional transcriptional control sequences as well. The vectors and expression cassettes may further comprise selectable markers.

The expression cassette will include in the 5′-3′ direction of transcription, a transcriptional and translational initiation region, a DNA sequence of the invention, and a transcriptional and translational termination regions. The transcriptional initiation region, the promoter, may be native or analogous, or foreign or heterologous, to the host cell. By “heterologous” or “foreign” is intended that the transcriptional initiation region (e.g., a promoter) is not found in the organism into which the transcriptional initiation region is introduced.

The subject invention also provides for the expression of a polypeptide, peptide, fragment, or variant encoded by a polynucleotide sequence disclosed herein comprising the culture of a host cell transformed with a gene of the subject invention under conditions that allow for the expression of the polypeptide and, optionally, recovering the expressed polypeptide.

In certain embodiments, the subject composition further comprises a metallic electrocatalyst, which aides in the production of soluble CO₂ intermediates that the oleaginous microorganisms can utilize for the production of lipids.

An electrocatalyst is a type of catalyst that functions at electrode surfaces, or as an electrode surface itself, to modify and increase the rate of electrochemical reactions through interaction with reagents to alter the reaction pathways and decrease the activation barrier.

In certain embodiments, the electrocatalyst comprises one or more of the fourth, fifth, and/or sixth period transition metals (Sc, Ti, V, Cr, Mn, Fe, Co, Ni, Cu, Zn, Y, Zr, Nb, Mo, Tc, Ru, Rh, Pd, and Hf) and/or their alloys. In some embodiments, a electrocatalyst can be doped with at least one naturally occurring element to improve the activity of the catalyst.

In certain embodiments, the electrocatalyst comprises low-microbe toxicity metallic species. Preferably, the electrocatalyst comprises Cu doped with Zn.

The electrocatalyst can be expressed using the formula CuₓZn_{y}, where X and Y can each be any positive integer between 1-10. In certain preferred embodiments, the electrocatalyst is Cu₆Zn₁.

Advantageously, use of the subject electrocatalyst can facilitate the tuning of the soluble CO₂ intermediate profile to optimize lipid production. For example, in certain embodiments, the Cu₆Zn₁ electrocatalyst can surprisingly generate optimal acetate/ethanol ratios, i.e., higher acetate-to-ethanol ratio, for lipid production using an engineered Gram negative and/or Gram positive organisms as described herein (e.g., Rhodoccocus jostii RHA1 expressing or overexpressing each of fasI, atf2, sthA and dmpF).

### Methods and Systems for Lipid Production

The subject invention further provides methods for producing a lipid biodiesel feedstock wherein the methods comprise an electrocatalysis step and a bioconversion step, said electrocatalysis step comprising converting CO₂ into CO₂ intermediates using an electrocatalytic CO₂ reduction reaction (CO₂RR); and said bioconversion step comprising converting the CO₂ intermediates into lipids using an oleaginous microorganism as described herein.

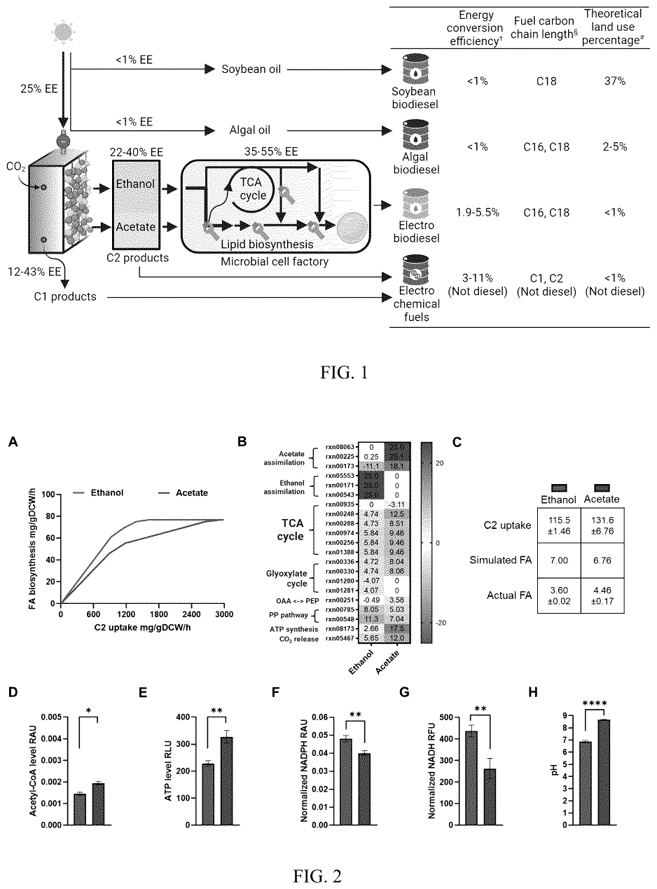

More specifically, in certain embodiments, the electrocatalysis step comprises supplying electricity and CO₂ to an electrocatalyst according to the subject invention in the presence of an alkaline medium, thereby generating CO₂ intermediates, such as, for example, ethanol and/or acetate, via a reduction reaction (see, for example, FIG. 1 or FIG. 6E). In further preferred embodiments, the method generates both ethanol and acetate at a ratio of 0.5 to 2.0, acetate/ethanol.

The electrocatalyst can operate at, for example, a current density of about 50 to 150 mA/cm², or about 75 to 125 mA/cm², or about 100 to 115 mA/cm².

The bioconversion step then comprises inoculating a growth medium with an oleaginous microorganism according to the subject invention, and supplying the growth medium with the CO₂ intermediates produced using the electrocatalyst, thus allowing the microorganism to grow and synthesize lipids from the CO₂ intermediates into the growth medium.

The microbial inoculant according to the subject methods preferably comprises cells and/or propagules of the desired microorganism, which can be prepared using any known fermentation method. The inoculant can be pre-mixed with water and/or a liquid growth medium.

The bioconversion step can further involve providing aeration to the growing culture. One embodiment utilizes slow motion of air to remove low oxygen-containing air and introduce oxygenated air. The oxygenated air may be ambient air supplemented daily through mechanisms including impellers for mechanical agitation of the liquid, and air spargers for supplying bubbles of gas to the liquid for dissolution of oxygen into the liquid.

The pH of the culture should maintained at neutral to alkaline values, preferably greater than 5.0, even more preferably greater than or equal to 7.0. Buffers, and pH regulators, such as carbonates and phosphates, may be used to stabilize pH near a preferred value: however, in certain embodiments, the engineered microorganism can modulate the pH of the growth medium due to expression of the dmpF and/or sthA genes. In certain embodiments, a base solution is used to adjust the pH of the culture to a favorable level, e.g., a phosphate. The base solution can be included in the growth medium and/or it can be fed into the reactor during cultivation to adjust the pH as needed.

In one embodiment, the bioconversion is carried out at about 5° to about 100° C., about 15° to about 60° C., about 20° to about 45° C., about 22° to about 35° C., or about 24° to about 30° C. In one embodiment, the bioconversion may be carried out continuously at a constant temperature. In another embodiment, the bioconversion may be subject to changing temperatures.

In certain embodiments, the method can then further comprise extracting the lipids from the growth medium and generating biodiesel from the lipids using known methods. For example, in some embodiments, the lipids can be subjected to transesterification to produce a biodiesel component comprising mostly fatty acid methyl esters. In certain embodiments, the biofuel product, e.g., fatty acid methyl esters, can be mixed with a carrier, such as water or a fossil fuel, to produce a finished biofuel product. The biofuel products can be utilized as biodiesel-diesel mixtures, biodiesel-water mixtures, pure biodiesels, and other suitable formulations for powering standard engines, diesel engines, marine engines, heaters, jet engines, generators, and heavy machinery.

Further provided herein is a system for continuous production of lipids from CO₂, which uses the compositions and methods of the subject invention to integrate electrocatalysis and bioconversion (see, for example, FIGS. 1, 6A, 6E and/or 23). In certain embodiments, the system comprises a two-chamber bioconversion unit (i.e., a bioreactor) and a CO₂ electrolysis unit (i.e., an electrolyzer) containing the bimetallic electrocatalyst.

During electrocatalysis, the electrolyzer, and thus, the electrocatalyst, is connected to an electrical supply and a CO₂ supply. In certain preferred embodiments, electricity is supplied to the system using solar power.

The electrocatalyst can be affixed to a surface or suspended in a fluid. Growth medium is circulated between the CO₂ electrolyzer and the first chamber of the bioconversion unit to accumulate CO₂ intermediates such as ethanol, acetate, propanol and formate, resulting from reduction reactions between the CO₂ and the electrocatalyst. The microbes are kept in the second chamber of the bioconversion unit, and separated from the first chamber using a non-selective filter membrane (e.g., a filter membrane having a pore size between about 0.1 μm and about 1 μm, about 0.1 μm to about 0.45 μm, or a filter membrane having a pore size of about 0.22 μm or about 0.3 μm). The filter membrane allows the CO₂RR products to diffuse from the first chamber to the second chamber during bioconversion, while efficiently blocking microbes from entering the first chamber and the electrolyzer. The oleaginous microbe is thus provided access to the CO₂ intermediates, allowing for growth and production of lipids into the culture medium of the second chamber.

The bioconversion reactor used according to the subject invention can be any fermenter or cultivation reactor for industrial use. As used herein, the term “reactor,” “bioreactor,” “fermentation reactor” or “fermentation vessel” includes a fermentation device consisting of one or more vessels and/or towers or piping arrangements. Examples of such reactor includes, but are not limited to, the Continuous Stirred Tank Reactor (CSTR), Immobilized Cell Reactor (ICR), Trickle Bed Reactor (TBR), Bubble Column, Gas Lift Fermenter, Static Mixer, or other vessel or other device suitable for gas-liquid contact.

In one embodiment, the reactor may have functional controls/sensors or may be connected to functional controls/sensors to measure important factors in the cultivation process, such as pH, oxygen, pressure, temperature, agitator shaft power, humidity, viscosity and/or microbial density and/or metabolite concentration.

In some embodiments, the chambers of the bioconversion reactor can each have a liquid loading volume of about 1 L to about 100 L, or about 5 L to about 50 L, or about 10 L to about 35 L, or about 15 L to about 25 L.

In preferred embodiments, the filter membrane of the bioconversion reactor comprises a 0.1 to 0.45 micron filter comprised of non-woven or woven textile, glass/quartz wool, fiber (cellulose, glass quartz, plastics, resins), honeycomb structured cellulose, polymer, metal, ceramic, activated or porous carbon, natural fibers (e.g., cellulose, cotton), resin materials (plastics) such as polypropylenes (PP), polyethylenes (e.g., polyethylene [PE], polyethylene terephthalates (PET), polytetrafluoroethylenes (PTFE), polyvinylidene fluorides, polyimides and polyamide-imides, perfluoralkoxy polymer resins, fluorinated ethylene propylene (FEP), ethylene tetrafluoroethylene (ETFE), and the like.

### Reduced Carbon Footprint Biofuel Production

The subject invention provides a highly productive, efficient, cost-friendly, and reduced-carbon footprint electro-biodiesel route that directly uses CO₂ as feedstock to fulfill industrial demands for diesel with significantly reduced land usage. Such land usage does not have to be arable lands, thus substantially alleviating food-energy competition and the shortage of biodiesel feedstock. Thus, the subject invention has the potential to revolutionize diesel-dependent sectors such as mining, drilling, shipping, and construction, which face substantial challenges in transitioning from fossil fuels.

In certain embodiments, the subject invention provides methods for reducing the carbon footprint of producing biofuels, particularly biodiesel, wherein a composition, method and/or system described herein is utilized in the production of the biofuels.

A “carbon footprint” may be defined as a measure of the total amount of carbon dioxide (CO₂) and other GHGs emitted directly or indirectly by a human activity or accumulated over the full life cycle of a product or service. As just one example, a product that requires transportation over many miles by truck (e.g., a fossil fuel) may have a larger carbon footprint than an alternative product that does not require transportation (e.g., a locally-produced biofuel).

Carbon footprints can be calculated using a Life Cycle Assessment (LCA) method, or can be restricted to the immediately attributable emissions from energy use of fossil fuels. A life cycle assessment (LCA, also known as life cycle analysis, ecobalance, and cradle-to-grave analysis) is the investigation and valuation of the environmental impacts of a given product or service caused or necessitated by its existence. The life cycle concept of the carbon footprint means that it is all-encompassing and includes all possible causes that give rise to GHG emissions. In other words, all direct (on-site, internal) and indirect emissions (off-site, external, embodied, upstream, downstream) need to be taken into account.

Normally, a carbon footprint is expressed as a CO₂ equivalent. Carbon dioxide equivalency is a quantity that describes, for a given mixture and amount of GHG, the amount of CO₂ that would have the same global warming potential (GWP), when measured over a specified timescale (generally, 100 years). Carbon dioxide equivalency thus reflects time-integrated radiative forcing. The carbon dioxide equivalency for a gas is obtained by multiplying the mass and the GWP of the gas. The following units are commonly used:
a) By the UN climate change panel IPCC: billion metric tons of CO₂ equivalent (GtCO₂ eq):
b) In industry: million metric tons of carbon dioxide equivalents (MMTCDE):
c) For vehicles: g of carbon dioxide equivalents/km (gCDE/km).

For example, the GWP for methane is 21 and for nitrous oxide 310. This means that emissions of 1 million metric tons of methane and nitrous oxide, respectively, is equivalent to emissions of 21 and 310 million metric tons of carbon dioxide.

Various methods exist in the art for calculating or estimating carbon footprints and may be employed in the practice of the subject invention.

Advantageously, in preferred embodiments, the subject invention can be useful for reducing the carbon footprint of producing biofuels, which includes reducing the carbon footprint of producing plant-based biofuel feedstock, forage-based, fodder-based and/or grain-based feed for animal-based feedstock, and transportation thereof.

A “reduced carbon footprint” means a negative alteration in the amount of carbon dioxide and/or other GHGs emitted per unit time over the full life cycle of producing feedstock and producing biofuels with said feedstock, through and until a biofuel is ultimately used by human consumers. The negative alteration in CO₂ and/or other GHG emissions can be, for example, at least 0.25%, 0.5%, 1%, 5%, 10%, 15%, 20%, 25%, 30%, 35%, 40%, 45%, 50%, 55%, 60%, 65%, 70%, 75%, 80%, 85%, 90%, 95% or 100%.

In some embodiments, the term “carbon footprint” is interchangeable herein with the terms “carbon intensity” and “emission intensity.” Emission intensity is the measure of the emission rate of a given GHG relative to the “intensity” of a specific activity or industrial process (e.g., burning of fuel, production of livestock animals, production of dishwashers). The emissions intensity can include the amount of emissions relative to, for example, the amount of fuel combusted, number of livestock animals produced, the amount of an industrial product produced, the total distance traveled, and/or the number of economic units generated.

Emissions intensity is measured across the entire life cycle of a product. For example, the emissions intensity of fuels is calculated by compiling all of the GHG emissions emitted along the supply chain for a fuel, including all the emissions emitted in exploration, mining, collecting, producing, transporting, distributing, dispensing and burning the fuel.

In certain embodiments, methods of the subject invention further comprise conducting measurements to assess the effect of the method on reducing the generation of carbon dioxide and/or other deleterious atmospheric gases, and/or precursors thereof (e.g., nitrogen and/or ammonia), using standard techniques in the art.

In certain embodiments, assessing GHG generation can take the form of measuring GHG emissions from a site. Gas chromatography and electron capture are commonly used for testing samples in a lab setting. Measuring GHG emissions can also comprise other forms of direct emissions measurement and/or analysis of fuel input. Direct emissions measurements can comprise, for example, identifying polluting operational activities (e.g., fuel-burning automobiles) and measuring the emissions of those activities directly through Continuous Emissions Monitoring Systems (CEMS). Fuel input analysis can comprise calculating the quantity of energy resources used (e.g., amount of electricity, fuel, wood, biomass, etc., consumed) determining the content of, for example, carbon, in the fuel source, and applying that carbon content to the quantity of the fuel consumed to determine the amount of emissions.

The application also provides the following non-limiting embodiments:

1. A composition for use in the production of diesel fuel lipid feedstock from CO₂, wherein the composition comprises an oleaginous microorganism, an electrocatalyst and a growth medium.

2. The composition of embodiment 1, wherein the oleaginous microorganism expresses or overexpresses one or more of the following enzymes: type 1 fatty acid synthase (FASI), diacylglycerol acyltransferase (DGAT), soluble pyridine nucleotide transhydrogenase and/or acetaldehyde dehydrogenase.

3 The composition of embodiment 1, wherein the oleaginous microorganism's genome comprises fasI and atf2.

4. The composition of embodiment 1, wherein the oleaginous microorganism's genome comprises sthA and dmpF.

5. The composition of embodiment 1, wherein the microorganism's genome comprises each of fasI, atf2, sthA and dmpF.

6. The composition of embodiment 1, wherein the microorganism is a Rhodococcus sp. selected from R. opacus and R. jostii.

7. The composition of embodiment 1, wherein the microorganism is Rhodoccocus jostii RHA1, and wherein the microorganism's genome is engineered to comprise each of fasI, atf2, sthA and dmpF.

8. The composition of embodiment 1, wherein the growth medium comprises ethanol and acetate as sole carbon sources.

9 The composition of embodiment 1, wherein the electrocatalyst has a formula expressed as CuₓZn_{y}, wherein X and Y are each any positive integer between 1-10.

10. The composition of embodiment 9, wherein the electrocatalyst is Cu₆Zn₁.

11. A method for producing a lipid biodiesel feedstock, the method comprising an electrocatalysis step, said electrocatalysis step comprising supplying electricity and CO₂ to an electrocatalyst to produce CO₂ intermediates via a reduction reaction between the CO₂ and the electrocatalyst; and a bioconversion step, said bioconversion step comprising inoculating a growth medium with an oleaginous microorganism, supplying the growth medium with the CO₂ intermediates produced using the electrocatalyst, and allowing the microorganism to grow and synthesize lipids from the CO₂ intermediates into the growth medium, wherein oleaginous microorganism that expresses or overexpresses one or more of the following enzymes: type 1 fatty acid synthase (FASI), diacylglycerol acyltransferase (DGAT), soluble pyridine nucleotide transhydrogenase and acetaldehyde dehydrogenase.

12. The method of embodiment 11, wherein the electrocatalyst comprises a formula CuₓZn_{y}, wherein X and Y are each any positive integer between 1-10.

13. The method of embodiment 12, wherein the electrocatalysis operates at a current density of 100 to 125 mA/cm².

14. The method of embodiment 11, wherein CO₂ intermediates comprise ethanol, acetate, propanol and/or formate.

15. The method of embodiment 14, wherein the ethanol and acetate are generated at a ratio of 0.5 to 2.0, acetate/ethanol.

16. The method of embodiment 11, wherein the microorganism's genome comprises each of fasI, atf2, sthA and dmpF.

17. The method of embodiment 11, wherein the oleaginous microorganism is Rhodoccocus jostii RHA1, and wherein the microorganism's genome is engineered to comprise each of fasI, atf2, sthA and dmpF.

18. The method of embodiment 11, wherein the growth medium is inoculated with the microorganism at a cell density of 4 to 5 g/L.

19. The method of embodiment 11, further comprising extracting the lipids from the growth medium and generating a biodiesel from the lipids.

20. A system for continuous production of lipids from CO₂, the system comprising a two-chamber bioconversion unit and a CO₂ electrolysis unit containing an electrolyzer and an electrocatalyst.

21. The system of embodiment 20, wherein the CO₂ electrolysis unit is connected to an electrical supply and a CO₂ supply.

22. The system of embodiment 21, wherein the electrical supply is solar powered.

23. The system of embodiment 20, wherein the two-chamber bioconversion unit comprises a first chamber and a second chamber separated by a 0.1 to 0.45 micron filter membrane, and wherein the first chamber is connected to the electrolysis unit.

24. The system of embodiment 23, wherein the first and second chambers are bioreactors having a liquid loading volume of 1 to 100 L each.

25. The system of embodiment 20, wherein the second chamber comprises a growth medium and an oleaginous microorganism, and wherein the filter membrane prevents the microorganism from entering the first chamber and the electrolysis unit.

26. The system of embodiment 25, wherein the system comprises an oleaginous microorganism that expresses or overexpresses one or more of the following enzymes: type 1 fatty acid synthase (FASI), diacylglycerol acyltransferase (DGAT), soluble pyridine nucleotide transhydrogenase and acetaldehyde dehydrogenase.

27. The system of embodiment 26, wherein the system comprises an oleaginous microorganism that expresses or overexpresses the following enzymes: type 1 fatty acid synthase (FASI), diacylglycerol acyltransferase (DGAT), soluble pyridine nucleotide transhydrogenase and acetaldehyde dehydrogenase.

28. The composition or method of any preceding embodiment, wherein the oleaginous microorganism expresses or overexpresses one or more of the following enzymes: type 1 fatty acid synthase (FASI), diacylglycerol acyltransferase (DGAT), soluble pyridine nucleotide transhydrogenase and acetaldehyde dehydrogenase.

29. The composition or method of embodiment 28, wherein the oleaginous microorganism that overexpresses the following enzymes: type I fatty acid synthase (FASI), diacylglycerol acyltransferase (DGAT), soluble pyridine nucleotide transhydrogenase and acetaldehyde dehydrogenase.

### Materials and Methods

### Rhodococcus Growth Under Ethanol and Acetate Conditions and Physiological Assay

A single colony of Rhodococcus strain on an LB agar plate was inoculated into a 10 mL LB medium and incubated overnight at 30° C. and 180 rpm. The cells were washed twice with phosphate buffered saline (PBS) and transferred to 50 mL Rhodococcus growth medium with an initial OD₆₀₀ at about 0.26 to carry out the cell growth assay. The Rhodococcus growth medium contains (per liter of deionized water): 1.7 g KH₂PO₄, 9.8 g Na₂HPO₄, 0.1 g MgSO₄, 0.95 mg FeSO₄·7H₂O, 10.75 mg MgO, 2.0 mg/L CaCO₃, 1.2 mg ZnSO₄, 0.2 mg CuSO₄, 0.15 mg CoSO₄·7H₂O, 0.06 mg H₃BO₄, and 51.3 ml HCl.⁵⁶ 24 mmol/L (NH4)₂SO₄ was added for the nitrogen source supply. To investigate the potential of C2 substrates for lipid production, ethanol or acetate were added into the Rhodococcus growth medium as the sole carbon sources. Considering high concentration of ethanol or acetate can be toxic to microorganisms^{57.58} and to mimic the electro-bio system, a feeding method of 45 mmol/L ethanol or acetate per 12 hours was adopted to supply carbon source. The cell culture was incubated at 30° C. and 180 rpm for 54 hours. The OD₆₀₀ and pH of the Rhodococcus culture were measured every 12 hours to monitor the cell growth and pH change. At the end-point of cell growth, cells were harvested from the culture by centrifugation. The cell pellets are then lyophilized for 24 hours for dry cell weight (DCW) and lipid measurement. The supernatant from the culture was collected for soluble CO₂RR substrate concentration measurement by ¹H NMR (Bruker AVANCE NEO 400) with D₂O as the solvent and DMSO as the internal standard.¹⁵

For lipid production, the washed R. jostii RHA1 cells were transferred into Rhodococcus growth medium with an OD₆₀₀ about 1.0. A feeding method of 90 mmol/L ethanol or acetate per 12 hours was adopted to supply carbon source with a limited 2 mmol/L (NH4)₂SO₄ supplementation to enhance lipid accumulation²⁶. The fermentation process was carried out for 36 hours followed by cell harvest and lyophilization.

As for the ATP, NAD(P)H, and acetyl-CoA assay measurement, R. jostii RHA1 cells suspended in the PBS were adjusted to the concentration with OD₆₀₀ at about 1.0, and then inoculated into 10 mL of the Rhodococcus growth medium supplemented with 45 mmol/L ethanol or acetate and 24 mmol/L (NH4)₂SO₄, at a 1% (v/v) inoculation ratio. The culture was incubated at 30° C. and 180 rpm. When OD₆₀₀ reached about 0.4, the cells were sampled for ATP, NAD(P)H, and acetyl-CoA assay.

### Genome-Scale Metabolic (GSM) Model and Flux Balance Analysis (FBA)

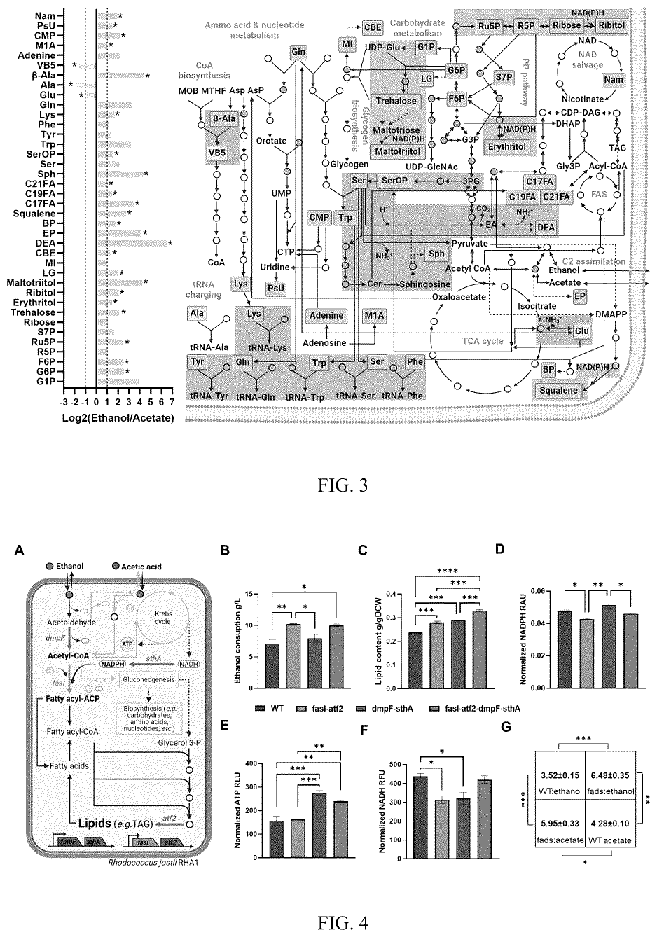

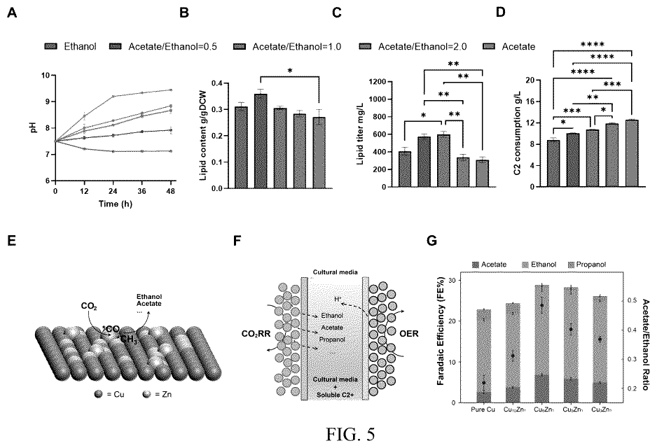

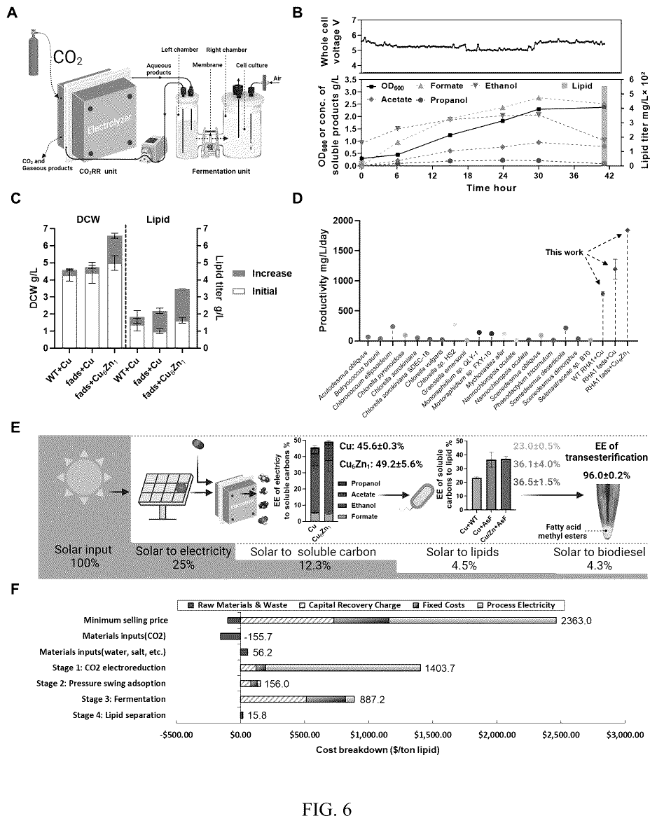

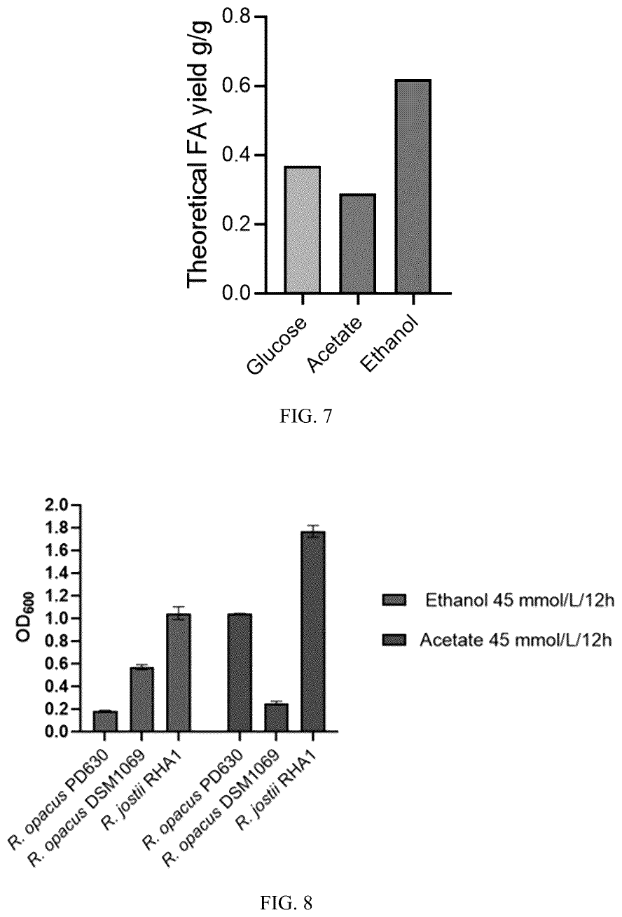

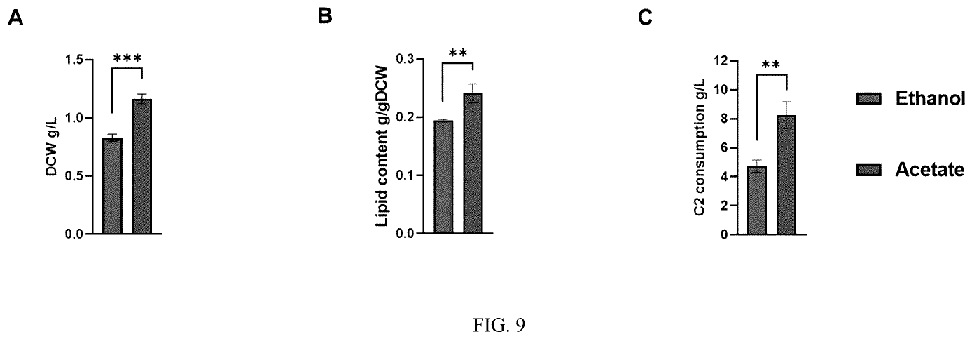

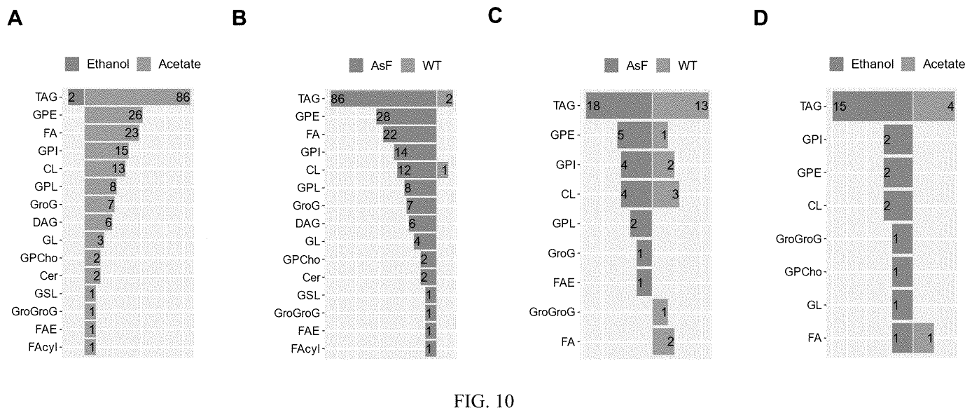

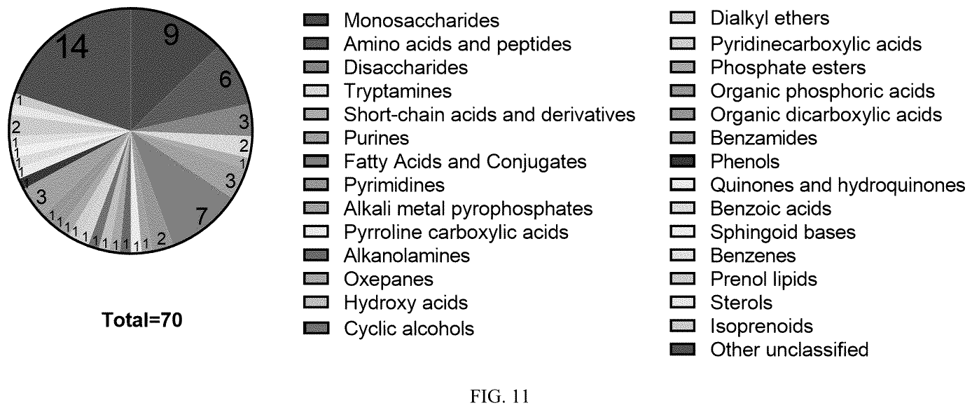

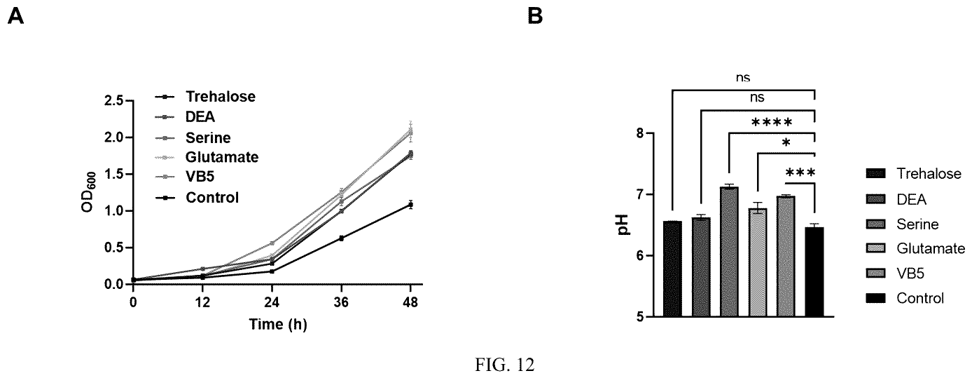

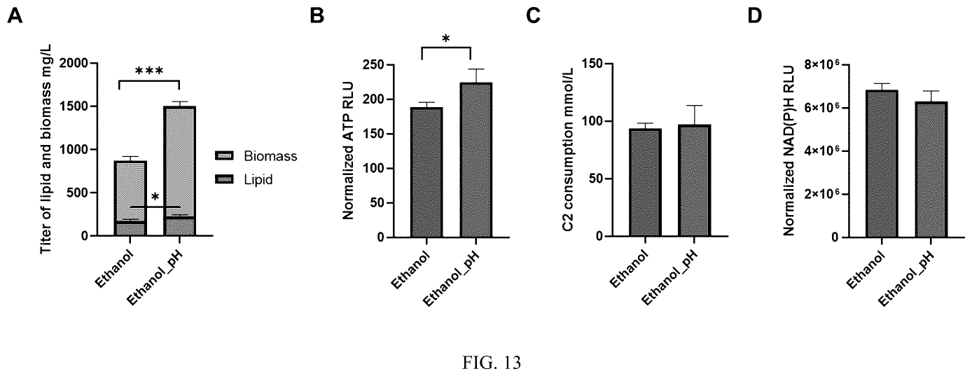

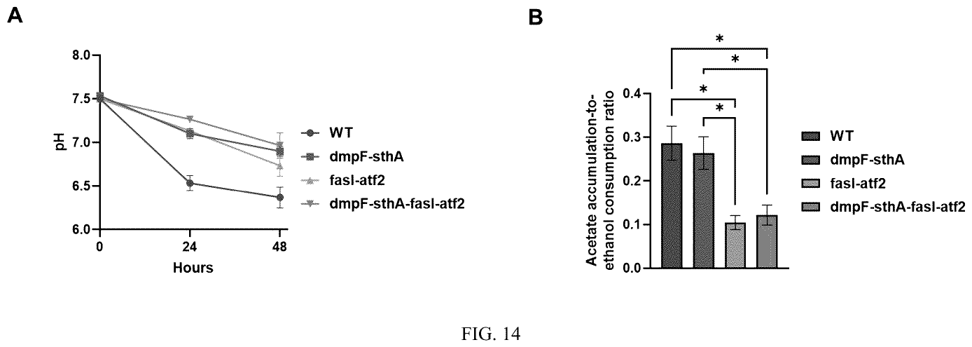

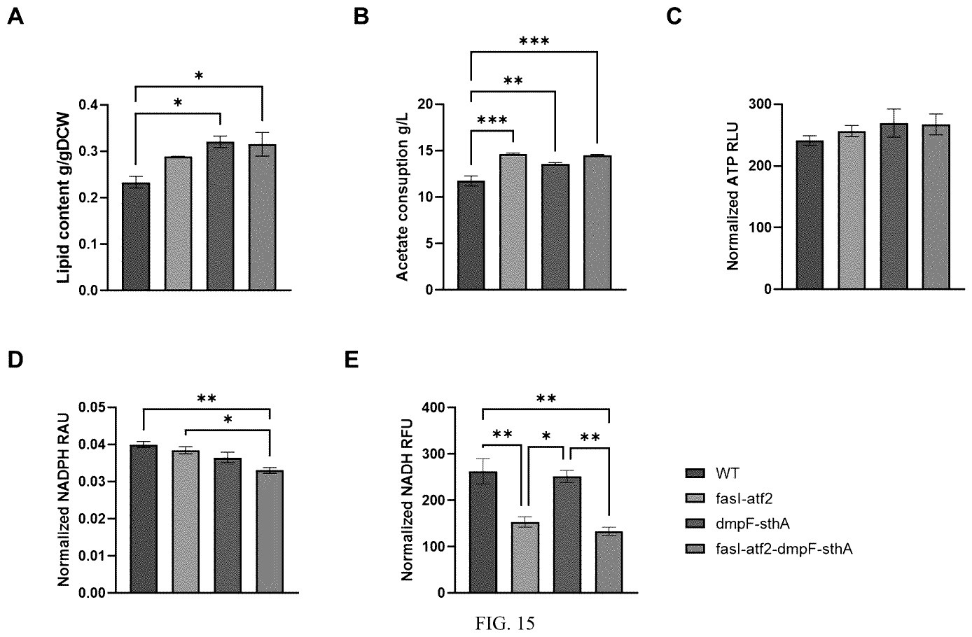

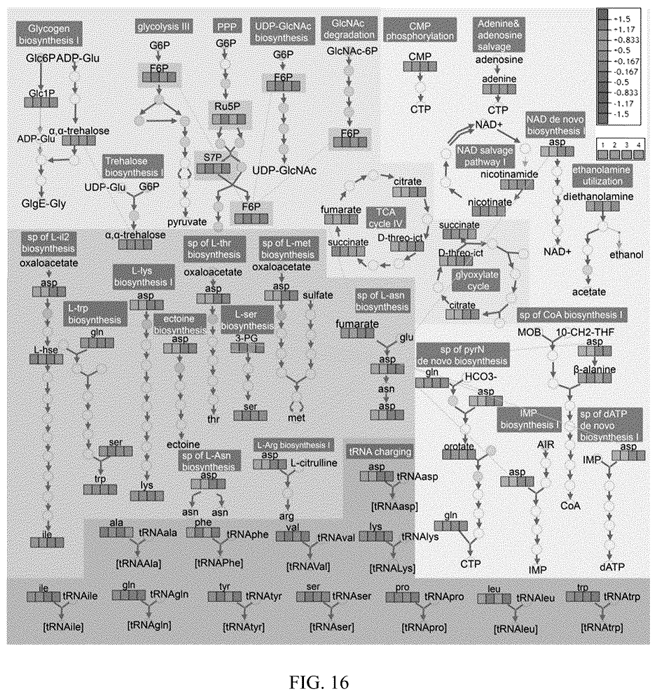

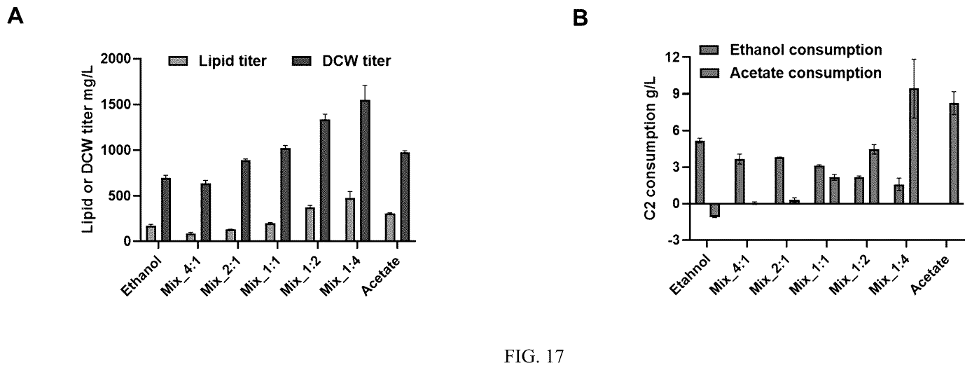

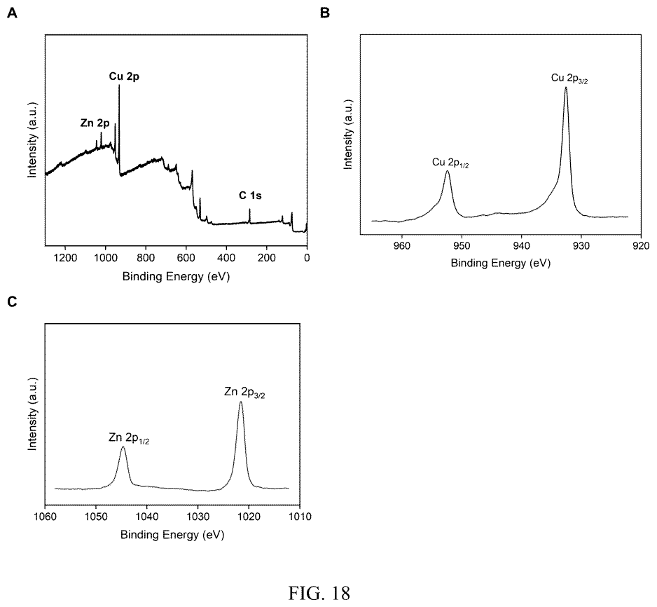

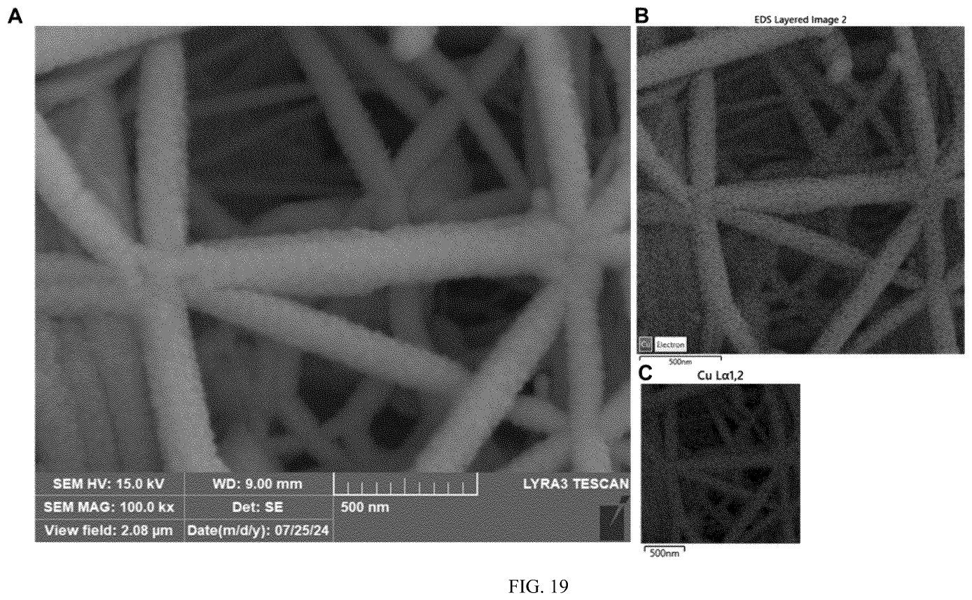

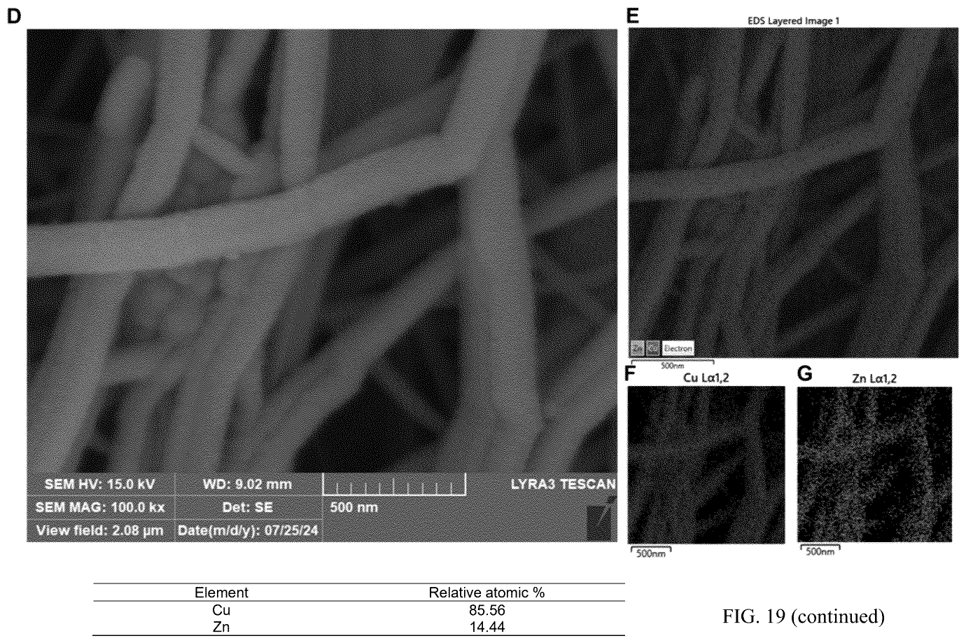

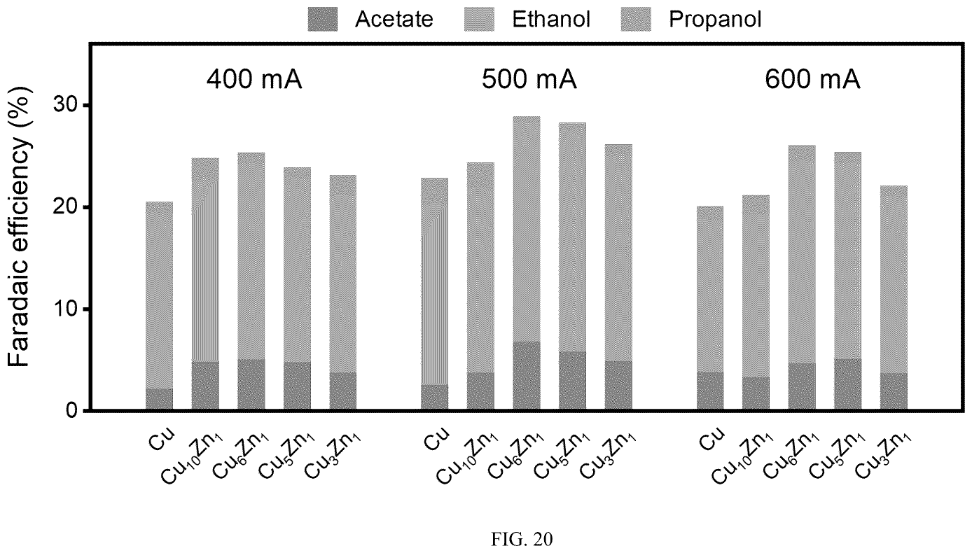

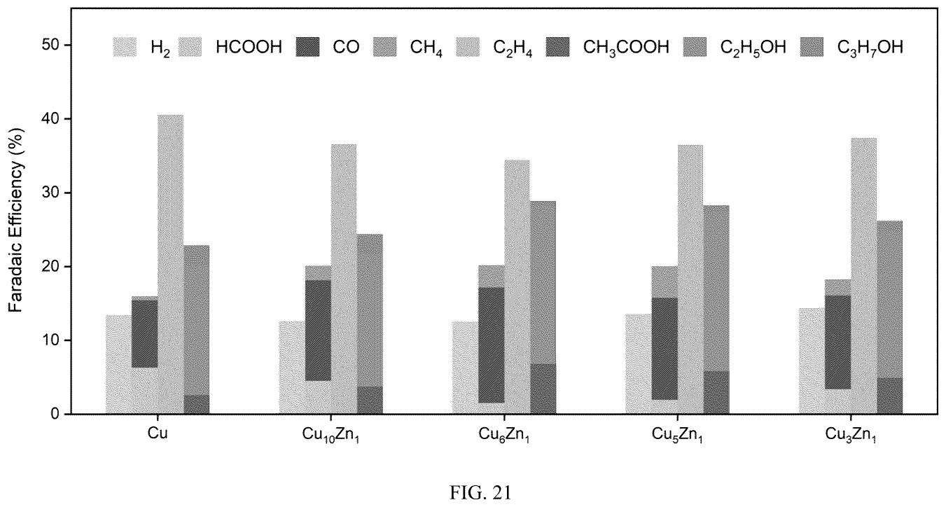

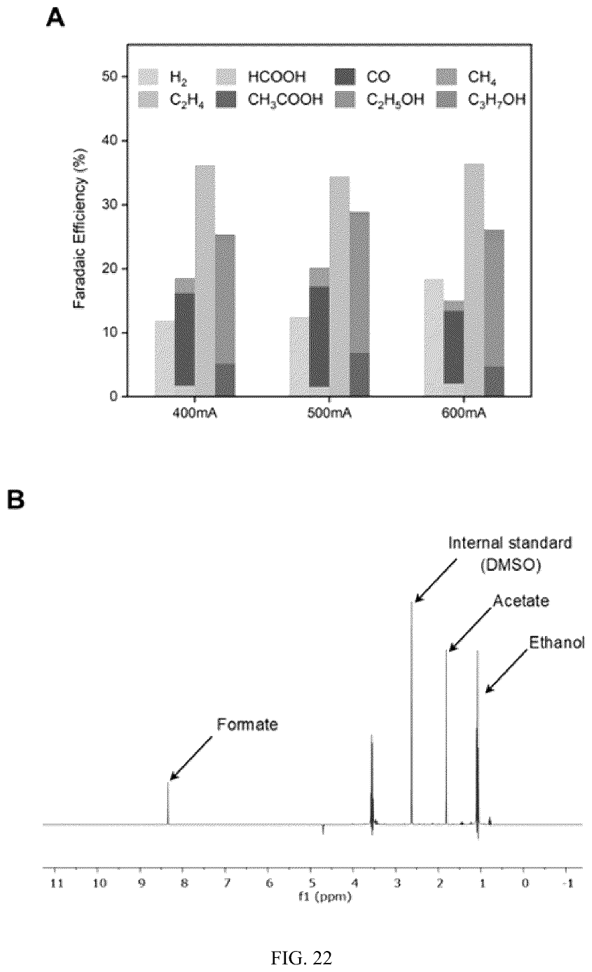

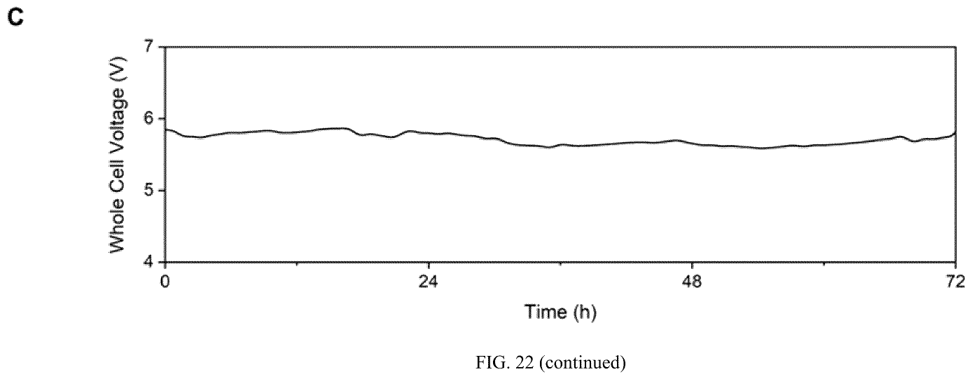

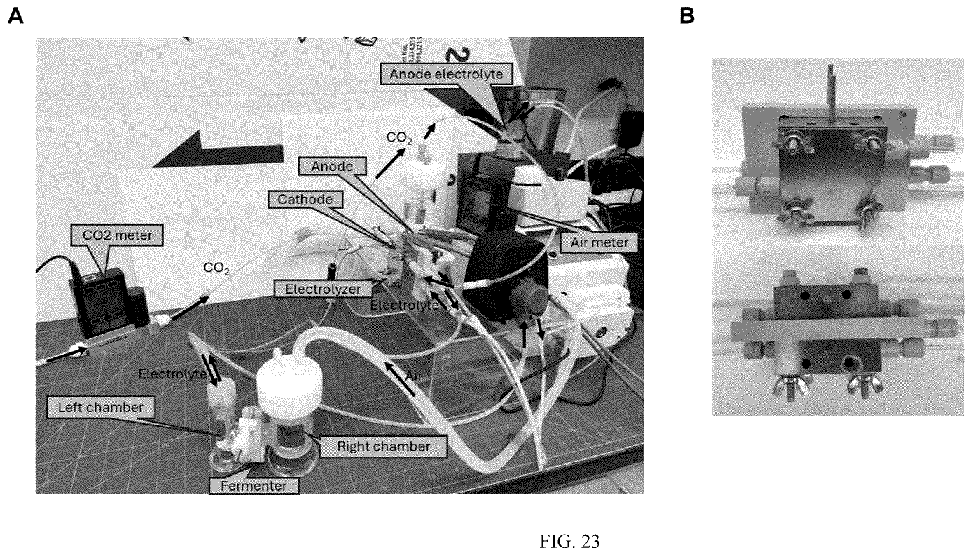

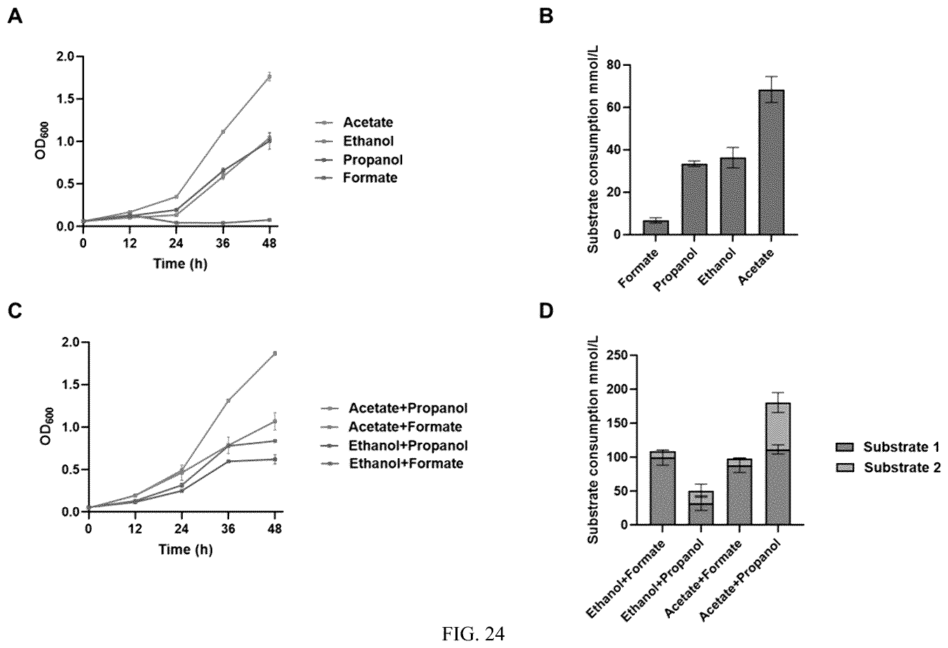

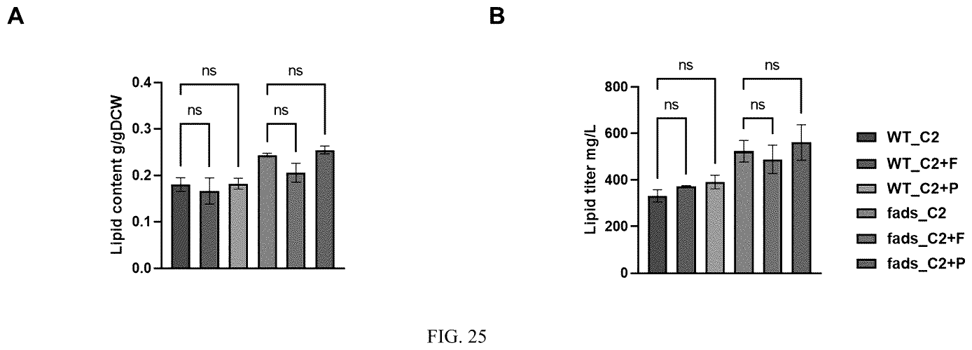

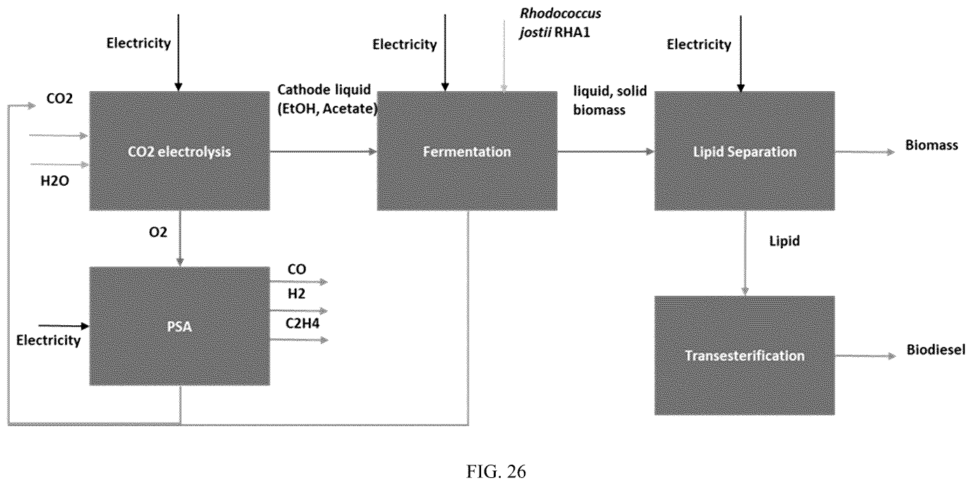

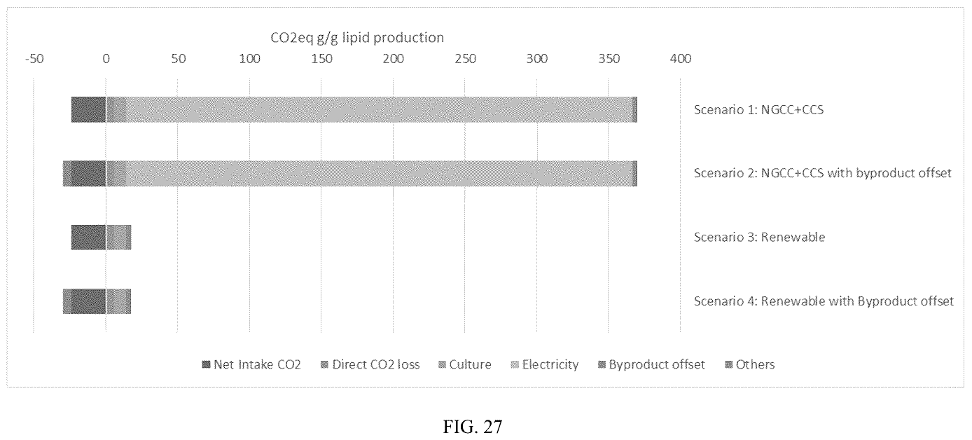

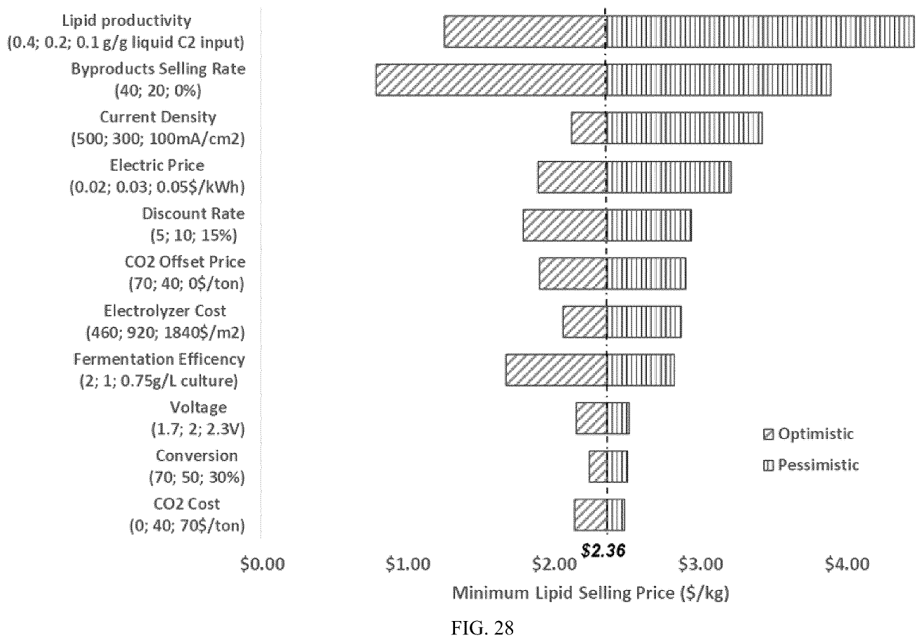

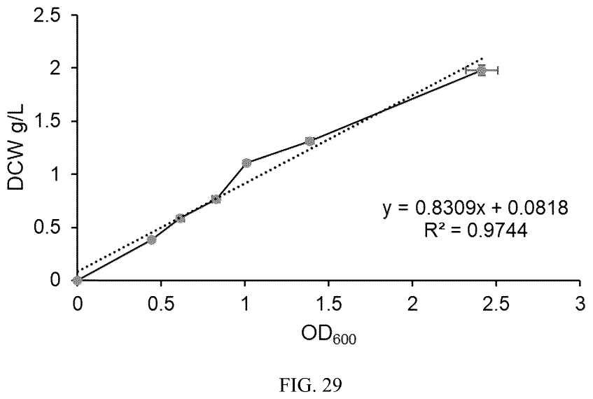

The KBase web-tool ^{23,59} was employed to conduct the GSM model construction and FBA analysis. Briefly, the complete genome of Rhodococcus jostii RHA1 was obtained from the KBase database and was annotated by means of the RAST (Rapid Annotation Subsystem Technology) tool in the platform. The annotated genome was used to generate a draft genome-scale metabolic model of RHA1 with standard parameters including an in-built gap-filling algorithm in the KBase. Additionally, the customized Rhodococcus medium with either ethanol or acetate as sole carbon source was used as the media file for construction of the model, RHA1modelPos Ethanoll Acetatel. The tool of Run Flux Balance Analysis was used to predict metabolite fluxes in the metabolic model of RHA1 grown on the customized Rhodococcus medium. Fatty acid biosynthesis was set as objective function for the FBA with a series of carbon uptake rates to simulate fatty acid production under different carbon inputs (FIGS. 2A and B).

### R.Jostii RHA1 Strain Construction for Improved Lipid Production

To improve the capacity of the RHA1 to convert ethanol to lipid, we constructed two plasmids to genetically modify the lipid biosynthesis pathway. The Rhodococcus engineering followed a previous the established protocol with some modifications. 17 The plasmids, strains, and primers used in the study are listed in Table 8 and Table 9.

The first plasmid is PDD-120-dmpF-ArtRBS-sthA to overexpress the dmpF (gene coding acetaldehyde hydrogenase from Rhodococcus jostii RHA1) and the sthA (gene coding hydrogen transferase from Escherichia coli str. K-12 sub strain MG1655) in the strain. Specifically, dmpF′ and sthA were amplified by PCR from R. jostii RHA1 genomic DNA and E. coli str. K-12 sub strain MG1655 genomic DNA, respectively. PDD 120 vector was produced by PCR amplification to remove Che9c60 and Che9c61 gene fragments from the PDD120 plasmid.⁶⁰ The PDD 120 vector, dmpF and sthA containing ribosomal binding sites (RBS) were fused by overlapping PCR to produce the PDD-120-dmpF-ArtRBS-sthA plasmid (Table 8).

The second plasmid is PBSNC9031-fasI-atf2-dmpF-sthA. Specifically, the fused gene fragment dmpF-sthA containing the constitutive promoter and RBS sites was amplified from the PDD-120-dmpF-ArtRBS-sthA plasmid via PCR. The FASI (Type I fatty acid synthase from Rhodococcus opacus PD630) and the PBSNC9031 vector were amplified from the PBSNC9031-Pben-FAS plasmid16 via PCR. The dmpF-sthA, FASI, and PBSNC9031 vector were assembled via Gibson method to produce the PBSNC9031-fasI-atf2-dmpF-sthA plasmid. Both plasmids underwent thorough verification by undergoing full plasmid sequencing conducted by Primordium Labs in the USA. Subsequently, engineered strains were created by introducing these plasmids into the RHA1 WT strain through the electroporation method.

### Cu/Zn Catalyst Manufacturing

The Cu catalyst was prepared by sputtering copper onto a porous PTFE membrane (0).45 μm, Tisch Scientific) using Kurt J. Lesker PRO Line PVD 7515. The Cu—Zn bimetallic catalysts were prepared by co-sputtering copper and zinc simultaneously. The thickness of the catalyst layer was about 200 nm by controlling the deposition time at 1041 seconds. After sputter deposition, we tested the conductivity of the catalyst layer by pinning any two points on the surface using a multimeter and making sure all the values were less than 1 Q. The bimetallic samples were denoted as CuxZny, where x:y represents the actual atomic ratios between Cu and Zn measured by SEM-EDS (FIG. 19).

### Operation of the Integrated Electro-Biodiesel System

In the integrated EBF system operation, the electrolyzer, where the CO₂ electroreduction takes place, was connected to a customized bioreactor consisting of two chambers. A phosphate-based minimal solution (the Rhodococcus growth medium) was used as electrolyte for CO₂RR and buffer solution for microbial processes. The left chamber (L) had a liquid loading volume of 15 mL, while the right chamber (R) had a liquid loading volume of 35 mL. The CO₂RR liquid products was circulated between the meddle chamber of the electrolyzer and left chamber of the bioreactor by a pump The CO₂RR liquid products were allowed to diffuse through a membrane from the left chamber to the right chamber, where the RHA1 cells use the CO₂RR products for bioconversion (FIG. 6A, FIG. 23). The copper catalyst operates at a current density of 100 mA/cm², while the Cu/Zn catalysts operate at a current density of 125 mA/cm².

The RHA1 cells are grown in the integrated system in two ways. First, we inoculate WT RHA1 cells in the integrated system at a low cell density with a nitrogen rich media (24 mmol/L (NH₄)₂SO₄) and monitor the growth curve to demonstrate the biocompatibility and biomass conversion performance of the system (FIG. 6B). Second, we introduce a relatively high cell density of RHA1 strains with a controlled high C/N ratio (2 mmol/L (NH₄)₂SO₄) to rapidly and efficiently produce lipid from CO₂ in the integrated system (FIGS. 6C, D, and E).

Specifically, in the first way, WT RHA1 was inoculated into the right chamber of the fermentation unit (initial OD₆₀₀ at about 0.3, FIG. 6B). The cell-culture compartment was put under 300 rpm, 30° C. for 42 hours. Cell density indicated by the OD₆₀₀ was monitored during the whole process using SpectraMax iD5. At the end point of time, the cells in the right chamber were collected by centrifuge (3000×g, 10 min), washed, and lyophilized to measure DCW and lipid content. The media in both chamber A and B was sampled across the 42-hour process to quantify the product profiles via 1H NMR (FIG. 6B). The normalized concentration of the product profiles was calculated as following formula:

$$Normalized conc {.}_{product} ( e . g . ethanol ) = \frac{( 15 mL * conc {.}_{product} in L + 35 mL * conc {.}_{product} in R )}{50 mL}$$

In the second way, WT or engineered fads was utilized as a whole cell catalyst to convert CO₂RR products to lipid. Specifically, RHA1 cells prepared from overnight LB culturing were inoculated into the right chamber of the bioreactor with an initial OD₆₀₀ at a range around 3.5 or 4.5 to achieve high cell density fermentation. The system operated for 24 hours, with the lipid fermentation process carried out throughout this period while the CO₂RR process performed only during the first 15 hours. This approach was adopted to align the CO₂RR product productivity and consumption rate, considering the relatively slow diffusion rate from the left chamber to the right chamber of the bioreactor. By performing the CO₂RR process in the initial 15 hours, it allowed sufficient time for the CO₂RR products to diffuse and reach the right chamber where the microorganisms were present for bioconversion. At the beginning (zero-hour) and after 24 hours, the cells were harvested for measuring DCW and lipid content (FIG. 6C). Additionally, the media was collected at these time points to measure the concentration of CO₂RR products, which was essential for calculating the energy efficiency.

Data shown in FIG. 7 are based on the following calculation. The energy conversion efficiency of each process to produce biofuel, electro-fuel, and electro-biofuel is referenced from literature. The operational photosynthetic energy conversion efficiency of for microalgal cultivation is about 1%. Most crops convert sunlight and CO₂ into plant biomass at an energy efficiency of 1% or less. The overall energy conversion efficiency from solar to algal and soybean oils is thus less than 1% (FIG. 1). The achievable energy conversion efficiency of photovoltaic technology in practical applications can reach 25% of energy efficiency. Selective CO₂ electroreduction to methanol can reach a faradic efficiency at 80%, which is up to 43% of energy conversion efficiency according to the potential difference between cathode and anode. Selective conversion of CO₂ to methane achieved a faradaic efficiency exceeding 70% under industrial current density conditions, resulting in an energy efficiency of approximately 12% with their applied cell potential. The half-cell cathodic energy efficiency of ethanol chemical productions from electrochemical CO₂ reduction can reach about 22%. The advanced CO₂-to-acetate electroreduction reached about 40%. The energy efficiency of converting solar energy into C1 or C2 fuels such as methanol, methane, and ethanol thus falls within the range of 3-11% (FIG. 7). C2 feedstocks such as ethanol and acetate can support an energetic efficiency of 35-55% for microbial cell growth. The estimated energy conversion efficiency of solar-electro-microbial biomass thus falls in the range of 1.9-5.5% (FIG. 7).

### Land Use Evaluation of Different Biodiesel Platforms

Data shown in FIG. 7 are based on the following calculation. The U.S. diesel consumption is 3.7 million barrels per day, which is referenced from EIA report, equating to 8,276,728,320,000 MJ/year. For the electro-biodiesel production platform, its electricity input ₁s sourced from maturing photovoltaic technology, which requires as low as 5.000 m² land per year to generate 1 GWh electric power. It means the power production rate is about 200 kWh/m2/year, equating 2,913,739 MJ/acre per year. Based on the assumption that land use of electro-biodiesel platform is mainly contributed by photovoltaic panel with the electro-biodiesel system being integrated under solar panel without extra land use, the energy productivity per unit land of the electro-biodiesel can be calculated by multiplying the electric power production rate by energy efficiency (EE) of downstream steps, including 49.2% EE of electricity-to-C2. 37.0% EE of C2-to-lipids, and 94.6% EE of lipid-to-biodiesel (see below section Calculation of energy efficiency for the Electro-biodiesel system), resulting in 501.774 MJ/acre per year. To fulfill the annual demand of diesel, it needs 16.5 million acres, which accounts for about 0).83% of the total land area (1.996.7 million acers) of the continental 48 states of the U.S. (Table 2). For algal biodiesel approach, the reported achievable annual production is 12.00 gallons/acre/year, according to NERL29, equating 175.104 MJ/acre/year, which thus requires about 47.3 million acres of land to meet the total diesel production (Table 2). For the soybean biodiesel approach whose annual production is around 50.6 bushels per acre according to the USDA Crop Production 2023 Summary. Based on a yield of approximately 1.5 gallons of biodiesel produced per bushel of soybean, one acre of land produces estimably 75.9 gallons of soy-diesel per year, which translates into 11,075.3 MJ/acer/year. It thus needs 747 million acres to meet the annual diesel consumption in the USA (Table 2).

The global biodiesel consumption is approximately 65.86 million tons, equating 3,003,216×10⁶ MJ. It's reported that each hectare of land (2.47 acres) can produce around 2.9 tons of palm oil. Given that 0.991 million tons of palm oil can yield 1.078 billion liters of palm methyl ester, one acre of land can produce approximately 1,276.6 liters of palm-diesel annually, translating into 49,481 MJ/acre/year, calculated by using diesel's energy content of 45.6MJ/kg and density of 0.85 kg/liter. Hence, it would require 60.7 million acres of land. In comparison, with an electro-biodiesel system capable of producing 530,417 MJ/acre/year, only 5.66 million acres would be needed to meet current global biodiesel consumption, representing less than 10% of the current land usage. More importantly, this land will not come with the deforestation of rain forest and does not need to be arable land.

### Cell Growth Monitoring and pH Measurement

For every 12 hours, 200 μL of cell culture were taken from the culture media and transferred into one well of a 96-well plate, and then the absorbance at wavelength 600 nm was measured with path check using a plate reader SpectraMax iD5 (Molecule Device, USA). The reads are referred to as optical density OD₆₀₀ in this work to indicate the cell growth. And the relation between OD₆₀₀ and DCW is shown in FIG. 8. For pH measurement, a 1.0 mL culture medium was taken from a 50 mL culture medium to a 15 mL culture tube for pH measurement using a pH meter (Apera Instruments, USA). All the data are collected with three biological replicates.

### ATP Measurement

Cellular ATP level is critical factor impacting fatty acid biosynthesis and lipid production in oleaginous microorganism such as Rhodococcus. ATP measurement was performed using the Sigma-Aldrich ATP Assay Kit (MAK135) according to the manufacturer's instructions. Specifically, biological triplicates of 10 mL of RHA1 cells were grown in Rhodococcus media with 45 mmol/L ethanol or acetate as sole carbon source in advance. ATP reagent for the assay was prepared according to the kit protocol and 90 μL of the ATP reagent solution was transferred into appropriate wells in in a 96-well plate. RHA1 cells at OD₆₀₀ about 0.2 indicating the cells had passed the lag phase and began to grow were sampled for cellular ATP measurement. Cells were prepared by dilution with PBS to OD₆₀₀ at around 0.1 and 10 μL of the cell suspension was transferred into appropriate wells. After gently mixing, the plate was incubated at room temperature for 10 min before luminescence measurement by SpectraMax iD5 (Molecule Device, USA). Reading of luminescence (relative light units) was normalized by the dilution factors and cell density (OD₆₀₀) of the cell sample to compare the relative ATP level between different samples (FIGS. 2E and 4E).

### NAD(P)H Level Measurement

Given that ethanol and acetate have different energy contents and yield varying amounts of reducing power during their metabolism, we employed a fluorescence-based method to measure the levels of NAD(P)H (NADH and NADPH) between the two C2 conditions (FIG. 13D). Specifically, biological triplicates of 10 mL of RHA1 wild-type and engineered cells were grown in Rhodococcus media with 45 mmol/L ethanol or acetate as sole carbon source in advance. 200 μL of cells were collected and used for NAD(P)H measurement. SpectraMax iD5 (Molecule Device, USA) was used for fluorescence signal measurement. The 340 nm was used for excitation wavelength, and the fluorescence emission at 460 nm wavelength was measured. Then the Reading of fluorescence was normalized by cell density (OD₆₀₀) and used to compare NAD(P)H level between samples.

### NADPH and NADH Measurement

The supply of NADPH and NADH is an important factor to support lipid production in oleaginous Rhodococcus.³⁴ The selective quantification of NADPH was done using a NADP+/NADPH Assay Kit (MAK312, Sigma). Briefly, RHA1 cells grow in the Rhodococcus media with 45 mmol/L ethanol or acetate as sole carbon source at log phase (OD₆₀₀ in range between 0.4 and 0.5) was diluted to OD₆₀₀ at around 0.2. Then the cells from three biological replicates were incubated with 80 μL of working reagents with enzymatic probes for specific detection of NADPH. Absorbance for NADPH quantification (OD₅₆₅) was measured at minute 0 and 30 using a TECAN (Molecule Device, USA) plate reader. The absorbance signal readings were normalized by cell density to allow comparison between samples. The resulting relative absorbance units (RAU) were compared between the WT and engineered strains (FIGS. 2F and 4D).

Similarly, the selective quantification of NADH was performed using the NAD+/NADH Assay Kit (MAK460), Sigma). The cells were lysed and then incubated with 50 μL of working reagents with enzymatic probes for the specific detection of NADH. Fluorescence for NADH quantification (ex=530 nm/em=585 nm) was measured at minute 0) and 10 using a TECAN (Molecule Device, USA) plate reader. The fluorescence signal readings were normalized by cell density to allow comparison between samples. The resulting relative fluorescence units (RFU) were compared between the WT and engineered strains (FIGS. 2G and 4F). Each sample was analyzed in three biological replicates.

### Acetyl-COA Measurement

Acetyl-CoA is a crucial intermediate product with a primary role in transporting the acetyl group into the Krebs cycle, where it is oxidized for energy production. The selective quantification of acetyl-CoA was performed using the Acetyl-CoA Colorimetric Assay Kit (E-BC-K652-M, Elabscience®). RHA1 cells grow in the Rhodococcus media with 45 mmol/L ethanol or acetate as the sole carbon source and were diluted to OD₆₀₀ at around 0.4. The cells were lysed and then incubated with 230 μL of working reagents with enzymatic probes for the specific detection of acetyl-CoA. Absorbance for acetyl-CoA quantification (OD₃₄₀) was measured at minute 0 and 1 using a TECAN (Molecule Device, USA) plate reader. The absorbance signal readings were normalized by cell density to allow comparison between samples. Each sample was analyzed in three biological replicates, and all samples were measured immediately or incubated according to different stages of the measurement. The resulting relative absorbance units (RAU) were compared between the WT and engineered strains (FIG. 2D).

### Lipid Extraction, Transesterification, and Quantification

The RHA1 cells were harvested through centrifugation at 5000 rpm for 10 minutes. Following that, the cells underwent two washes with ddH₂O and were subsequently lyophilized for 2 days. Total DCW of each sample was measured with analytic balance (Sartorius, Michigan). Approximately 5-10 mg of the lyophilized cells were dissolved in a mixture of 2 mL methanol-sulfuric acid (v/v=85:15) and 2 mL chloroform. The resulting solution was then incubated at 100° C. for four hours, during which acid-catalyzed methyl esterification converted the fatty acids of lipids into their corresponding methyl esters, which were dissolved in chloroform. After cooling, the samples were washed twice with 2 mL of demineralized water until no acid residual remained. The upper layer (water phase) was removed after each wash. The fatty acid methyl esters (FAME) are in the organic layer and can be obtained by evaluating the chloroform by a gentle stream of nitrogen in a fume hood.

For quantification of lipid production, the organic layer was then diluted with chloroform by 20 times and added with internal standard methyl benzoate with a concentration at 21 μg/mL. The final solution was filtered using a 0.2 μm filter before being analyzed by GC-MS (QP2010SE, Shimadzu) with a Zebron ZB-35HT Inferno column (30 m×250 μm ID×0.25 μm df). Helium was used as the carrier gas with a flow rate of 1.0 mL/min. The column temperature was programmed to start at 50° C. for 3 minutes and ramped to 300° C. at a rate of 10° C./min. The injector temperature was set to 250° C. Mass spectra were recorded with a 70 eV electron beam at an ionization current of 40 μA.

The quantification for each component was performed through calculating a response factor (RF) for each component analytical standard. For example, the RF for C16 monomer analytical standard was calculated using the following expression:

$$RF = ( {A}_{C 16} \times {C}_{in} ) / ( {A}_{in} \times {C}_{C 16} ) = ( {A}_{C 16} / {A}_{in} ) / ( {C}_{C 16} / {C}_{in} )$$

where A_{C16} is the peak area of C16 (palmitic acid) analytical standard, which was detected as methyl palmitate following transesterification, Aᵢₙ is the peak area of the internal standard methyl benzoate: C_{C16} and Cᵢₙ are the concentrations of the C16 analytical standard and internal standard methyl benzoate, respectively. Based on the peak area and concentration of internal standard, and the peak area of C16, the concentrations of C16 monomer in the 2 mL GC/MS sample were determined. The weight of lyophilized cells used for lipid extraction was then employed to calculate the lipid content in the RHA1 cells.

To analyze the transesterification yield, about 100 mg of palmitic acid (C16), the most abundant fatty acid in the lipid, was accurately weighed and dissolved in the mixture of 2 mL methanol-sulfuric acid (v/v=85:15) and 2 mL chloroform to go through the methyl esterification reaction. After washing the sample twice with demineralized water, the ester product was obtained by evaporating the organic layer in the fume hood overnight. The ester product was accurately weighed, and the reaction yield was calculated using the following formula:

$${Yield}_{transesterification} = \frac{weight of final ester product ( mg )}{theoredical weight of ester product ( mg )} \times 100 \%$$

where the theoretical weight of the ester product is the weight of methyl palmitate converted from 100 mg of palmitic acid by 100% methylation, which is 105.5 mg.

### Metabolomics Analysis of Primary Metabolism and Complex Lipids

Biological triplicates of RHA1 WT and fads (overexpression of fasI, atf2, dmpF, and sthA genes) cells cultured in Rhodococcus media with ethanol or acetate as sole carbon source were collected, immediately frozen with liquid N₂, and preserved in −80° C. freezer. The frozen samples were delivered in dry ice condition to the West Coast Metabolomics Center at University of California Davis to analyze metabolites. All the sample preparation, metabolites extraction and measurement, data acquisition and analysis were conducted at the UC Davis Metabolomics Center. Briefly. Gas chromatography time-of-flight mass spectrometer-mass spectrometry (GC-TOF-MS) in automated liner exchange cold injection system (ALEX-CIS) was used to detect primary metabolites. The sample preparation was performed utilizing their established techniques for metabolite profiling and while data acquisition employed the chromatographic parameters described in their previous study. Electrospray ionization quadrupole time of flight mass spectrometer tandem mass spectrometry (ESI QTOF MS/MS) was used to detect the complex lipids extracted from samples. The complex lipid were extracted by using the methyl-tert-butyl ether method. The general data processing workflow involved the use of MS-DIAL for initial processing, followed by blank subtraction in Microsoft Excel and data cleanup using MS-FLO. Peaks were annotated by manually comparing MS/MS spectra and accurate masses of the precursor ion to the spectra provided in the Fiehn laboratory's LipidBlast spectral library.

### Bioinformatics Analysis and Visualization

The peak intensities of all annotated chemicals were normalized based on the sample weights used for the analysis experiment. These normalized values of the identified metabolites were subsequently subjected to statistical analysis using MetaboAnalyst 5.0. Interquartile range (IQR) method and a threshold value of 5% was used for sample filtering. For the primary metabolism, fold change analysis was performed (WT_EtOH vs WT_AA, fads_EtOH vs fads_AA, WT_EtOH vs fads_EtOH, and WT_AA vs fads_AA) on all identified primary metabolites with default parameter setting to find significantly changed metabolites (FIG. 3A). To visualize the data, the values of the metabolites were standardized and mapped onto the metabolic pathways based on the R. jostii RHA1 representative genome in BioCyc database (biocyc.org/overviews Web/celOv.shtml?orgid=GCF_000014565 #) (FIG. 3A). For the complex lipids, t-test analysis was performed on the identified lipid compounds with significance level of 0.05 to find significantly changed. These lipid compounds were further classified with enrichment analysis using the main class of chemical structures library in the MetaboAnalyst 5.0 platform.² The hit numbers of lipid compounds in each category were visualized as back-to-back bar plots with R studio (FIG. 3B).

### Electrochemical CO₂ Reduction

The electrochemical CO₂ reduction reactions (CO₂RR) were performed using the Autolab PGSTAT302N potentiostat/galvanostat (METROHM) in the two-electrode mode. A customized dual-membrane electrolyzer⁴³ was used to evaluate the reaction performance. The electrolyzer configuration is shown in FIG. 6A. On cathode side, a 4 cm² Cu—Zn gas diffusion electrode (GDE) with porous PTFE base was employed. 30 standard cubic centimeters per minute (sccm) of humidified CO₂ was fed on its backside to provide CO₂ and water vapor to cathode catalyst. An anion-exchange membrane (AEM, Sustainion X37-50 RT, Dioxide Materials) was masked on the cathode to avoid the cations deposition, while allowing liquid products transportation. On anode side, 4 cm² Ni foam was employed for water oxidation using 1 M KOH as the electrolyte. A bipolar membrane (BPM, Fumasep FBM, Fuel Cell Store) was masked on the Ni foam for cation exchange while spacing out the KOH solution from the middle chamber between the AEM and the BPM. A phosphate-based minimal solution (typically, the Rhodococcus growth medium) flew through the middle chamber at a rate of 5 ml min⁻¹ to elute the CO₂RR liquid products, while the salt composition in the solution provide conductivity for the whole electrolyzer. The solution was fixed to 20 mL and continuously circulated through the middle chamber to accumulate the liquid products.

Calculation of Faradaic Efficiencies (FEs) from Product Concentration

For all liquid samples, the concentration of soluble CO₂RR products was measured by 1H NMR using D₂O as the solvent and DMSO as the internal standard. The FEs of a specific product are calculated from its concentration using the following equation²⁵:

$${FE}_{product} = \frac{{c}_{product} \times {n}_{product} \times V \times F}{{J}_{total} \times t}$$

In this equation, c_{product} represents the concentration of a specific product, and n_{product} represents the number of electrons that one product molecule gets from CO₂ electroreduction. V is the solution volume. F is Faradaic efficiency, which is 96485 C/mol. Jₜₒₜₐₗ is the total current, while t represents the reaction time.

### Calculation of Energy Efficiency for the Electro-Biodiesel System

To calculate the energy efficiency from sunlight to final products, we analyzed the whole process and divided it into three stages, which are solar to electricity via photovoltaic technology (stage 1), electricity to CO₂RR liquid products (stage 2), and CO₂RR liquid products to lipid (stage 3) as depicted in FIG. 6E.

For stage 1, the energy conversion efficiency of the photovoltaic panel available from the market was approximately 25.0%. This efficiency value was utilized to estimate the overall efficiency of stage 1 in our system. For the energy efficiency of stage 2, we employed the approach introduced by Liu et al., wherein the energy content of the liquid products (energy output) was divided by the total energy input required to produce these liquid products (formate, ethanol, acetate, and propanol), which were subsequently used for lipid production in the WT and fads strains. The calculation can be expressed as follows:

$$EE = \frac{Δ {H}_{product} \times {R}_{product} \times t}{\sum ( {R}_{product} \times {n}_{product} ) \times F \times applied voltage \times t} = \sum {EE}_{product}$$

where the ΔH_{product} (kJ mol⁻¹) is the combustion heat of each liquid product, R_{product} (M/s) indicates the production rate of each liquid product, t(s) is the specific amount of time of the CO₂RR, n_{product} is number of electron of each liquid product, F is Faradaic coefficient, which is 96,485 C mol-1, The applied voltages (V) equated 2.6 V when using Cu catalyst and 3.6 V when using Cu₆Zn₁ catalyst, which was derived from the average cell voltage after iR compensation and then converted to the values relative to the reversible hydrogen electrode (RHE). The resistances (R) were determined by Electrochemical impedance spectroscopy (EIS) in the frequency ranging from 105 to 10-1 Hz and the amplitude was set at 10 mV.⁴³

In the electrocatalysis step (with the Cu₆Zn₁ catalyst as example), the production rate of each liquid product (R_{product}), number of electrons used (n_{product}), and their combustion heats (ΔH_{product}), and calculated energy efficiency (EE) are listed as follows:

| | R_{product} | | ΔH_{product} | EE_{product} |
|---|---|---|---|---|
| Product | (mM/L/h) | n_{product} | (kJ mol⁻¹) | (%) |
| Formate | 14.8 ± 0.8 | 2 | −254.6 | 5.2 ± 0.5% |
| Ethanol | 17.0 ± 1.1 | 12 | −1367.6 | 32.5 ± 4.1% |
| Acetate | 8.0 ± 0.1 | 8 | −875.1 | 9.8 ± 0.6% |
| Propanol | 0.6 ± 0.1 | 18 | −2021.3 | 1.7 ± 0.4% |

Therefore, the energy conversion efficiency of electricity to liquid products were:

$${EE}_{electricity to products} = 49.2 \%$$

This calculation is adopted because it excludes the energy consumption at the anode and internal resistance within the electrochemical system, making it more suitable for comparison with natural photosynthesis-driven compound synthesis processes. By considering the total electric energy consumption of the entire cell, rather than focusing solely on the energy used to produce liquid products, the energy efficiency of the whole electrochemical system for liquid products is:

$$EE = \frac{\sum ( Δ {H}_{product} \times {R}_{product} ) \times volume \times t}{Current \times Whole cell voltage \times t}$$

where the R_{product} (mmol/L/h) is the production rate of each liquid product in electrolyte solution with volume of 20 mL. The current applied is 500 mA, and the whole cell voltage is 6.5 V when using Cu₆Zn₁ catalyst. The energy efficiency of the whole CO₂RR catalysis cell is:

$${EE}_{electricity to products} = 21.7 \%$$

For stage 3, CO₂RR liquid products were used as the carbon source for the RHA1 strains to produce lipid. The fads strain was used as an example for calculation here. The energy efficiency was calculated as:

$${EE}_{products to lipid} = \frac{Energy {content}_{generated lipid}}{Energy {content}_{consumed products}}$$

To accurately evaluate the Energy content_{consumed products} during the lipid fermentation process, we assume the R_{product} is stable during the CO₂RR process to calculate the Amount_{total products} from CO₂RR, and we monitored the concentration of liquid products at the starting and end points of the fermentation to calculate the amount of unconsumed products (Product_{unconsumed}). Therefore, the Energy content_{consumed liquid products} is calculated as follows:

$$Energy {content}_{consumed products} = \sum ( ( {R}_{product} \times {t}_{CO 2 RR} \times volume - {Product}_{unconsumed} ) \times Δ {H}_{product} ) + C {2}_{initial} * Δ {H}_{C 2} = {Product}_{consumed} \times Δ {H}_{product}$$

where the R_{product} (mM/L/h) indicates the production rate of each liquid product, which is measured and calculated based in volume of 20 mL. The t_{CO2RR} is the CO₂RR process performed in the integrated system, which are 15 hours. The ΔH_{product} (kJ mol⁻¹) is the combustion heat of each liquid product. The C2ᵢₙᵢₜᵢₐₗ represents the externally added C2 carbon sources, which are 0.75 mmol ethanol and acetate, respectively. The ΔH_{C2} indicate the combustion heats of ethanol and acetate. The amount of unconsumed products (Product_{unconsumed}), production rate of each liquid product (R_{product}), and their combustion heats (ΔH_{product}) are listed as follows:

| | R_{product} | Product_{unconsumed} | Product_{consumed} | ΔH_{product} |
|---|---|---|---|---|
| Product | (mM/L/h) | (mmol) | (mmol) | (kJ mol⁻¹) |
| Formate | 14.8 ± 0.8 | 1.4 ± 0.7 | 3.1 ± 0.7 | −254.6 |
| Ethanol | 17.0 ± 1.1 | 1.5 ± 0.3 | 3.9 ± 2.5 | −1367.6 |
| Acetate | 8.0 ± 0.1 | 1.7 ± 0.2 | 1.5 ± 0.2 | −875.1 |
| Propanol | 0.6 ± 0.1 | 0.07 ± 0.02 | 0.07 ± 0.02 | −2021.3 |

Therefore,

$$Energy {content}_{consumed products} = 7.58 kJ$$

The Energy content_{generated lipid} can be calculated as follows:

$$Energy {content}_{generated lipid} = {Titer}_{lipid} \times {volume}_{lipid} \times Δ {H}_{lipid} = 2.77 kJ$$

where the Titer_{lipid} (mg/L) is the averaged lipid titer produced by the fads strain in the electro-biodiesel system, equals to 1840.2±3.3 mg/L (FIG. 6C), the is the volume_{lipid} is fads culture volume to lipid production in the electro-biodiesel system, which is 40 mL, the ΔH_{lipid} (kJ/g) the combustion heat of microbial lipids, which is 37.6 kJ/g.

Therefore,

$${EE}_{products to lipid} = \frac{Energy {content}_{generated lipid}}{Energy {content}_{consumed products}} \times 100 \% = 36.5 \pm 1.5 \%$$

Therefore, the overall solar-to-lipid energy efficiency (FIG. 6E) is:

$${EE}_{Solar to lipid} = 25 \% \times 49.2 \% \times 36.5 \% = 4.5 \%$$

For stage 4, microbial lipids are methylated to produce FAME, the final biodiesel product.

The energy content of the final product is referenced to microalgae FAME, which has a calorific value of up to 39.2 MJ/kg. The energy content of microbial lipids is about 37.6 MJ/kg. Methanol, required for the methyl esterification process, has an energy content of about 22.7 MJ/kg. Considering that complete transesterification of one mole of triglycerides (e.g. TG C16: 0/C16: 1/C18: 0, molecular weight 832 g/mol) consume three moles of methanol, generates one mole of glycerol and three moles of FAME (FAME C17, FAME C17, and FAME C19), then transesterification of 1 kg of lipid will consume about 0.12 kg methanol and generate about 1 kg FAME, thus,

$${EE}_{transesterification} = \frac{Energy content of FAME}{Energy content of lipid and methanol} \times {Yield}_{transesterification} = 96. \pm 0.2 \%$$

where the Yield_{transesterification} is experimentally determined from the transesterification process of palmitic acid (C16), which is the major component of fatty acid in the lipid obtained from RHA1 cells.

Specifically, the input C16 for methyl esterification obtained FAME and the yield of transesterification are as follows:

| | | Theoretical | Obtained | |
|---|---|---|---|---|
| Sample | Consumed | FAME | FAME | |
| number | C16 (mg) | obtains (mg) | (mg) | Yield_{transesterification} |
| 1 | 105.0 | 110.7 | 109.1 | 98.5% |
| 2 | 107.9 | 113.8 | 112.4 | 98.8% |
| 3 | 102.6 | 108.2 | 107.2 | 99.1% |

Overall,

$${EE}_{Solar to electro - biodiesel} = {EE}_{Solar to lipid} \times {EE}_{transesterification} = 4.3 \%$$

### Example 1—Metabolic Responses to C2 Intermediates During Microbial Lipid Biosynthesis Indicates Potential Bioenergetic and Metabolic Limits

To evaluate the potential of C2 intermediates for supporting lipid biosynthesis, theoretical yields of fatty acid (FA) synthesis from different C2 intermediates were calculated based on their energy contents as 0.62 g FA/g ethanol, 0.29 g FA/g acetate, and 0.37 g FA/g glucose, respectively (FIG. 7).18 This calculation shows that the energy content of C2 intermediates could sustain a comparable FA yield to that of glucose, particularly ethanol carrying high energy and more electrons. Nevertheless, microbial FA synthesis not just depends on substrate energy content but is also impacted by substrate metabolism. We, therefore, carried out genome-scale metabolic (GSM) modeling to evaluate the potential FA yield in ideal microbial metabolic process.

Rhodoccocci are known engineerable oleaginous microbial species to produce high lipid content under stress conditions from diverse substrates.^{17.19-21} We first carried out an experimental evaluation of multiple oleaginous species and strains for their capacity to grow in C2 intermediates. R. jostii RHA1 has shown superior performance when growing on both acetate and ethanol substrates as compared to R. opacus PD630 and DSM1069 (FIG. 8). To investigate the bioenergetic and metabolic capacity of FA synthesis from C2 intermediates in R. jostii, we constructed a draft GSM model based on KBase platform to evaluate the utilization of ethanol or acetate as the sole carbon source.^{22.23} Result of the flux balance analysis (FBA) shows that ethanol supports a higher FA biosynthesis yield than acetate, which is consistent with the theoretical FA yield calculation (FIG. 2A, FIG. 7). The results can be established from simulated metabolism (FIG. 2B). First, ethanol assimilation does not consume a significant amount of ATP like acetate assimilation (rxn00225).²⁴ Correspondingly, metabolism simulation with ethanol exhibits lower energy metabolism including ATP synthesis, tricarboxylic acid (TCA) cycle, and CO₂ release in the simulation (FIG. 2B). This represents a significant advantage over acetate as a substrate because low energy metabolism can reduce the diversion of carbon flux away from FA biosynthesis, as shown in the simulation.²⁵ Second. ethanol assimilation generates NADH, which can power NADPH generation, and other cellular activities. Specifically, the GSM simulation shows a higher pentose phosphate (PP) pathway and glyoxylate cycle (rxn01280 and rxn01281) in ethanol substrate than those in acetate, resulting in more NADPH to drive FA biosynthesis in Rhodococcus26 (rxn 10954 in FIG. 2B). Overall, the GSM simulation reveals that ethanol could be bioenergetically and metabolically more efficient in supporting FA synthesis than acetate in an optimal metabolic scenario. However, the GSM model includes 56 gap-filling reactions²³ and could mis-represent the C2 intermediate metabolism in the RHA1 strain. To validate the modeling outcome, we carried out the experimental analysis of lipid synthesis from C2 intermediates.

The experimental validation revealed surprising contradictory results to modeling. Specifically, the cell growth, lipid accumulation and substrate consumption of RHA1 in ethanol are all significantly lower than that in acetate (FIG. 9). In particular, the actual FA accumulation rate in ethanol is calculated to be merely 3.60±0.02 mg/gDCW/h, significantly lower than the rate of 4.46±0.17 mg/gDCW/h in acetate, contrasting with the simulation results based on the C2 consumption rates (FIG. 2C). These results indicate the presence of bioenergetic and metabolic limits impeding the conversion of ethanol into lipids, and the need for substantial engineering to realize the metabolic potential of the C2 compounds, in particular for ethanol utilization.

To identify these limits, we hence investigated the levels of substrate consumption, cellular acetyl-CoA level, ATP production, and NADH, and NADPH generation, which are the critical factors to affect lipid biosynthesis.²⁶ First, RHA1 strain exhibits a significantly lower ethanol consumption rate at 4.74±0.24 g/L than that of acetate at 8.25±0.54 g/L within a 54-hour period (FIG. 9C). The result is consistent with the lower acetyl-CoA levels on ethanol substrate as compared to that on acetate substrate (FIG. 2D). The lower ethanol consumption and acetyl-CoA level suggested a limited carbon flux entering lipid biosynthesis. Second. the RHA1 cellular ATP level on ethanol substrate was significantly lower than that on acetate substrate (FIG. 2E). This could be because the low ethanol uptake has limited the ATP production via energy metabolism.²⁷ Third, the levels of NADPH and NADH on ethanol substrate are both significantly higher than those on acetate substrate (FIGS. 2F and G). However, the imbalance between the high NAD(P)H level and the low ATP level suggested an inefficient conversion of NAD(P)H into ATP. Fourth, there is a significant decrease in culture media pH (FIG. 2H), which aligns with the observed acetic acid secretion. The result suggested that ethanol utilization could cause intracellular acidification stress. It could have impacted the cellular metabolism and lipid biosynthesis.

### Example 2—Metabolomics Analysis Revealed the Need to Rebalance ATP and Reductant Generation as Well as Overcome Acidification

To gain a deeper insight into the cellular metabolism related to the acetic acid accumulation and imbalance of ATP and reducing equivalents, we carried out metabolomics analysis to analyze the complex lipid species and primary metabolites from RHA1 growing on ethanol and acetate substrates. Lipidomic analysis identified 253 lipid species, revealing a significantly lower production level for 197 species under ethanol conditions, predominantly comprising triacylglycerols (TAG) and phospholipids (FIG. 10A). The results highlighted that the lower ATP level and the lower pH of the cell may have caused impaired lipid synthesis on ethanol substrates. Metabolomic analysis reveals that, out of 27 differential metabolites. 24 exhibit significantly higher levels when utilizing ethanol as the substrate. These up-regulated metabolites included a range of amino acids, nucleotides, and carbohydrate metabolites (FIG. 3. FIG. 11). These results notably unveiled the systemic metabolic response of RHA1 to acidification and the imbalance of reducing equivalents associated with ethanol utilization. First, the higher levels of amino acids and tRNA charging indicate an increased synthesis of proteins in RHA1 in response to enhance cellular acid tolerance (FIG. 3). 28.29 These amino acids can also undergo decarboxylation to consume intracellular protons (e.g. serine in FIG. 3), or undergo deamination to generate NH3 (e.g. glutamate in FIG. 3), to reduce cellular acidification. 29-31 To verify the potential mechanism, we have carried out a supplementing assay with amino acids including serine and glutamate. The serine and glutamate supplementation both increased cell growth and pH of RHA1 culture on ethanol substrate, supporting that the increase of amino acid could be cellular responses to acidification (FIG. 12). Since they are not basic amino acids, the pH increase is not due to their dissolution but rather their metabolism contributing to cell growth and acidification alleviation. Furthermore, the conversion of serine-derived ethanolamine into diethanolamine could also potentially generates more NH₃⁺ for acidification response, and diethanolamine also can promote cell growth of RHA1 with ethanol substrate as a carbon source (FIG. 12). Second, despite the overall decrease levels in lipid species under ethanol conditions, there are significant activities in the synthesis and degradation of the amino lipid ceramides (Cer), indicated by the significantly high levels of serine (Ser), D-erythro-sphingosine (Sph), diethanolamine (DEA), and fatty acids (FIG. 3). It corroborates with the previous findings that ceramide metabolism in Solibacter usitatus shifts with pH change, highlighting RHA1's cellular response to acidification stress during ethanol utilization. 32 Third, the trehalose accumulation in carbohydrate metabolism can enhance microorganisms' tolerance to acidification.^{33.34} The trehalose supplementation significantly improved RHA1 growth on ethanol without changing the pH of the media, supporting that trehalose might have increased tolerance to acidification rather than alleviating it (FIG. 12). Fourth, the relatively low level of pantothenic acid (VB5) together with the accumulation of β-alanine on ethanol substrate underscores a suboptimal activity of pantoate-β-alanine ligase, which prefers an alkaline pH for the catalysis to generate VB5 (FIG. 3). As the precursor of CoA, the low level of VB5 correlates with the observed low acetyl-CoA level on ethanol substrate (FIG. 2D). The VB5 supplementing thus improved RHA1 growth (FIG. 12). Overall, metabolomics revealed general stress responses to combat acidification, highlighting the necessity of synthetic biology to mitigate acidification for engineering electro-biodiesel production.

Besides acidification, metabolomics analysis revealed crucial need to balance reductant and ATP. There is a broad carbonyl reduction in diverse substrates, resulting in the buildup of maltotriitol, erythritol, ribitol, from ketoses and squalene from methylerythritol phosphate (MEP) pathway (FIG. 3). This carbonyl reduction and isoprenoid biosynthesis could be a strategy for RHA1 to consume excess reducing equivalents generated from ethanol assimilation. Collectively, the acidification and reducing equivalent imbalance issues of ethanol utilization cause the carbon diversion away from lipid biosynthesis to other metabolisms. Moreover, the concomitant low ATP level also needs to be addressed to drive lipid biosynthesis.

### Example 3—Metabolic Engineering to Enhance Lipid Biosynthesis from C2

Based on the modeling and metabolomics results, we designed the synthetic biology strategy to convert to lipid more efficiently from C2 intermediates. The simulation showed that ethanol carries more electrons and energy and thus could lead to more reductant and ATP production and could enhance lipid production under a scenario where ethanol's energy is effectively utilized for lipid precursor synthesis. However, the simulation does not reflect the challenges of acidification and reducing equivalent imbalance of ethanol utilization. The comparison between the simulated and real metabolic scenarios guides our metabolic engineering strategy development. First, the simulation shows more carbon uptake can increase fatty acid biosynthesis (FIG. 2A). However. RHA1 exhibited relatively low carbon uptake when utilizing C2 as a substrate, particularly for ethanol (FIG. 9C). Second, the simulation suggests the ethanol-to-acetaldehyde-to-acetyl-CoA as a primary pathway for ethanol assimilation, which neither costs ATP nor generates acetic acid (FIG. 2B). However, in real cellular metabolism, it appears that ethanol was quickly oxidized into acetic acid and caused the acidification and reductant imbalance (FIGS. 2F, G, and H). Therefore, it prompted us to externally adjust the pH during cell culturing on the ethanol substrate to test whether it could alleviate acidification and improve lipid production. The pH adjustment significantly alleviated acidification, leading to significant improvements in cell growth and cellular ATP levels (FIGS. 13A and B). However, the effect on lipid production was limited (FIG. 13A). This may be because pH adjustment did not significantly enhance carbon consumption, failing to increase carbon flux and reducing equivalents into lipid production, which highlights the necessity of metabolic engineering (FIGS. 13C and D).

We thus hypothesize that redirecting carbon from acetic acid generation to lipid biosynthesis can improve ethanol conversion into lipid. To verify the hypothesis, we overexpressed fasI operon coding type I fatty acid synthase (FASI) and atf2 gene coding diacylglycerol acyltransferase (DGAT) in RHA1 to channel more acetyl-CoA into fatty acid and lipid biosynthesis (FIG. 4A).¹⁷ FASI is a single large, multiunit, and multifunctional enzyme complex that can conduct the condensation and elongation of fatty acids with high efficiency.³⁵ DGAT catalyzes the final step in the biosynthesis of triacylglycerols (TAGs).¹⁷ Previous studies indicated that the co-expression of the two enzymes can lead to a significant increase in lipid productivity, whereas single gene over-expression will not achieve the effect. The overexpression of both genes significantly improved the ethanol uptake of RHA1 by 45.1%±18.7%, decreased the acetic acid accumulation by 63.8±0.8%, and resulted in an 18.0%±4.1% increase in lipid accumulation (FIGS. 4B and C. FIG. 14). Consistently, the engineered strain also showed a trend of improved lipid content when using acetate as a carbon source (FIG. 15A). These results suggest that the upregulation of FASI and DGAT effectively enhanced carbon flow from C2 to lipid biosynthesis. Interestingly, the fasI-atf2 strain exhibited significantly lower levels of NADPH compared to the wild-type (WT) strain, suggesting a higher level of NADPH is needed for FA biosynthesis (FIG. 4D).²⁶

We further explored strategies to balance reducing equivalents and reduce acidification. Gene sthA coding soluble pyridine nucleotide transhydrogenase was selected to increase NADPH supply to boost lipid production (FIG. 4A).36 Simultaneously, the gene dmpF encoding the acetaldehyde dehydrogenase was overexpressed to convert acetaldehyde into acetyl-CoA, mitigate the acetaldehyde-to-acetic acid oxidation, and supply NADH for sthA (FIG. 4A). 37 As a result, overexpression of the dmpF′ and sthA significantly improved the ATP level of WT RHA1 by 78.4%±25.8% (FIG. 4E). Since the overexpression significantly mitigated the medium acidification (FIG. 14A), the ATP improvement could be attributed to the less ATP cost on proton expelling for mitigating acidification stress. 31.38 The slight increase in NADPH level and significant decrease in NADH level indicates the conversion from NADH to NADPH by the transhydrogenase (sthA) (FIGS. 4D and F). Additionally, the lipid content in the dmpF-sthA strain increased by 21.6%±0.7% compared to WT RHA1 (FIG. 4C). These results highlighted the importance of ATP and NADPH supply for lipid biosynthesis from ethanol. Additionally, when this engineered strain uses acetate as carbon source, it also shows a significant increase in lipid content and acetate consumption, indicating that the metabolic engineering also improves acetate to lipid conversion (FIGS. 15A and B). There exists a trend of ATP level improvement compared to the WT strain on acetate substrate, but the NADPH was not improved as on ethanol substrate, likely due to the relatively low cellular NADH on acetate substrate. The low reductant level makes it insufficient to drive the catalytic reaction of the transhydrogenase (sthA) to generate NADPH (FIGS. 15C, D and E). The results indicated that we might consider leveraging the reducing equivalent produced from ethanol to drive the acetate conversion to lipid.

To improve NADPH and ATP supply for lipid biosynthesis in fasI-atf2 strain, we overexpressed dmpF′ and sthA in fasI-atf2 the strain. The overexpression of dmpF′ and sthA significantly improved the NADPH and ATP levels in fasI-atf2 strain by 48.0%±20% and 47.6%±3.5%, respectively (FIGS. 4D and E). Moreover, the lipid content in the fasI-atf2-dmpF-sthA (fads) strain reached 33.1=1.0% in dry cell weight (DCW), which was 39.4%±1.5% increase compared to the WT strain (FIG. 4C). Consistently, the lipid content reached 31.5±2.5% in the fads strain when using acetate as the sole carbon source, with an increase of about 35.4±10.3% compared to the WT strain (FIG. 15A). However, the NADPH and NADH level is significantly lower than that in WT strain, indicating the insufficient energy content of acetate to effectively drive lipid biosynthesis as compared to ethanol in the fads strain (FIGS. 15D and E). The results highlighted that reducing acidification and balancing reductant and ATP production has achieved more efficient conversion of C2 compounds, especially ethanol, into lipids, verifying the metabolic capacity as revealed in the simulation.

To verify the mechanism, the metabolomics analysis of the fads strain was conducted. The primary metabolism and lipid profiles in the fads strain are changed by metabolic engineering. Primary metabolites involved in carbohydrate metabolism, amino acid, and nucleoside biosynthesis were broadly decreased while levels of lipid species were increased in the fads strain under the ethanol condition, suggesting a carbon partition redirection into lipid biosynthesis by the metabolic engineering (FIGS. 16 and 10B). Notably, the decrease in the levels of trehalose and amino acids is consistent with the observation of mitigated acidification of fads under the ethanol condition (FIGS. 16 and 14). The metabolic engineering also improved lipid levels under acetate conditions (FIG. 10C). Interestingly, more lipid species are observed at higher levels when sourced from ethanol compared to acetate (FIG. 10D), which is consistent with the lipid production results of this strain, showing a higher fatty acid biosynthesis rate in ethanol than in acetate (FIG. 4G). Additionally, the strain exhibited a significantly higher fatty acid synthesis rate than the WT strain in both ethanol and acetate substrates (FIG. 4G), highlighting that metabolic engineering has overcome the metabolic and bioenergetic limits and enabled RHA1 to use C2 substrates, in particular ethanol, more effectively for lipid production.

### Example 4-Catalyst Design to Tune Acetate/Ethanol Ratio to Improve Electro-Biodiesel Efficiency

The synthetic biology data indicated that sthA-dmpF design could substantially increase ATP and NADPH production (FIGS. 4D and E). The engineered strain also unleashed the metabolic potential of ethanol. Considering that ethanol carries more electrons and has higher energy efficiency, it is probable that the reductant produced from ethanol can drive acetate conversion to lipid, to increase the overall carbon conversion efficiency. Another impact is that the co-substrate synergy could balance the acidification effect of ethanol (FIG. 2H). The verification of this co-substrate effect will lead to a new strategy to improve electro-biodiesel productivity through the co-design of electrocatalysts and microbial engineering. We therefore investigate the synergetic effect on lipid production with mixed ethanol and acetate substrates at various ratios to verify the hypothesis using the fads strain (FIG. 5). The results showed with a total C2 substrate concentration of 180 mmol/L, when the acetate/ethanol ratio reached 0).5 or higher, the acidification of culture could be significantly relieved (FIG. 5A). Consequently, the lipid content and titer of fads using the mixed substrates reached 0.36+0.02 mg/mg DCW and 599.4+34.4 mg/L, respectively, both higher than that using pure ethanol or acetate substrates (FIGS. 5B and C). The lipid titer of the fads strain peaked at C2 with the acetate/ethanol ratio at 1.0 despite a decrease in lipid content (FIGS. 5B and C). Notably, the carbon consumption at this ratio is significantly lower than sole acetate condition, suggesting the co-substrate drives carbon into lipid production more efficiently (FIG. 5D). This phenomenon implies that ethanol in the mixed substrate could have enhanced acetate conversion into lipids by providing reductants from its assimilation. Considering acetate assimilation requires ATP, future studies on converting ethanol-derived reductants into ATP could further enhance lipid biosynthesis from these co-substrates. Collectively, a mixed C2 with the acetate/ethanol ratio ranging from 0.5 to 1.0 supports a higher lipid production than sole ethanol or acetate. Notably, similar lipid production responses were also observed when mixed C2 substrates were used to support the growth of WT RHA1, highlighting the co-substrate synergetic effect of ethanol and acetate could be an effective strategy to achieve higher lipid production titer (FIG. 17).

Our previous work has established that copper catalysts can efficiently produce ethanol as its main soluble C2 product in a biocompatible phosphate solution. 15 Based on the co-substrate effect of ethanol and acetate in the fads, we seek to tune the soluble C2 product profile from CO₂RR to facilitate C2 to lipid conversion. The bi-metallic design could improve C2+ product Faraday Efficiency in strong alkaline solutions.³⁹ We have adapted the principle in designing a new bimetallic catalyst for phosphate buffer. The doped secondary metal could promote the synthesis of CO from CO₂, improve the adsorption of key intermediate *CO on the surface, and regulate the pathway of C—C coupling in C2 synthesis.³⁹⁻⁴³ Zn is chosen as the doping metal species, considering its low toxicity to microorganisms, high selectivity towards CO, and excellent compatibility with copper species. We hence developed a series of Zn-doped Cu catalysts (CuₓZn_{y}, x:y stands for the ratio between Cu and Zn, FIG. 5E) to tune the ratios between acetate and ethanol generated from CO₂RR, and used them to conduct CO₂RR in our three-chamber flow electrolyzer with phosphate electrolyte (FIG. 5F).¹⁵ The Cu₆Zn₁ bimetallic catalysts were prepared by co-sputtering Cu and Zn on the PTFE substrate with controlling the power of copper and zinc. The co-existence of Cu and Zn on the substrate was proved by XPS (FIG. 18) and SEM-EDS (FIG. 19).

Among our Cu/Zn catalysts, the Cu₆Zn₁ achieved the highest acetate-to-ethanol ratio of 48.5% at 125 mA cm-2 (FIGS. 5G, and 20, 21). Notably, this ratio of zinc doping also achieved a maximum FE of soluble C2+ at 28.9±0.5% (FIG. 5G). Considering it takes more electrons to produce one unit of ethanol (12 electrons) than acetate (8 electrons), the Cu₆Zn₁ catalyst with a higher acetate-to-ethanol ratio capacity rendered higher overall C2+ content compared to those catalysts that selectively produce ethanol with a comparable FE performance.⁴² It suggested the Zn-doped Cu catalyst design managed to generate optimal acetate/ethanol ratio for the lipid production with our fads strain. Additionally, the Cu₆Zn₁ catalyst maintained its performance for over 70 hours (FIG. 22), demonstrating excellent stability, and thus was used for integrated bioconversion.

### Example 5-Integrated Electro-Biodiesel Enabling Rapid and Efficient CO₂-to-Lipid Conversion

With the successful design of the catalyst and microorganism, we managed to integrate the CO₂RR and bioconversion processes in a continuous compatible system to produce lipids from CO₂. (FIGS. 6A and 23).¹⁵ The system is composed of a CO₂ electrolysis unit and a bioconversion unit. In the bioconversion unit, a two-chamber design was implemented. Part of the medium is circulated between the CO₂ electrolyzer and the left chamber to accumulate CO₂RR products, while the microbes are kept in the right chamber. A non-selective filter membrane was placed between the two chambers, which allows the CO₂RR products to diffuse to the right chamber, while efficiently blocking the microbes from entering the left chamber and the electrolyzer.

We first inoculated WT RHA1 into the integrated system to evaluate the biocompatibility and biomass conversion performance of the electro-biodiesel system (FIGS. 6A and B). The WT RHA1 cells in the right chamber utilized the soluble carbon sources diffused from the left chamber to support their growth and lipid biosynthesis effectively. The whole system maintained a stable C2 concentration level and rapid cell growth for 42 hours. Specifically, the voltage of electrolyzer remained constant between 5 and 6 volts across the time, and the concentrations of ethanol and acetate kept increasing in the first 30 hours, indicating the catalyst produced sufficient C2 substrates for RHA1 to use. A rapid RHA1 growth is observed, indicated by the optical density at 600 nanometers (OD 600) increasing from 0.3 to 2.2 (FIG. 6B). Moreover, the lipid titer increased from 32.4 mg/L to 562.1 mg/L within 42 hours, equivalent to a lipid productivity rate of 302.7 mg/L/day, which is comparable to the lipid productivity of high lipid-producing algae and microalgae (FIG. 6D).⁴⁴⁻⁴⁶ To further enhance the lipid production from CO₂ in this integrated system, RHA1 strains were introduced at a relative high cell density and with the control of high carbon/nitrogen ratio.²⁶ Different combinations of RHA1 strains and catalysts are tested in the integrated system to evaluate the lipid production (FIG. 6C). First, with 4.5±0.4 g/L of WT RHA1 cell density, the system equipped with Cu catalyst achieves 785.2±34.8 mg/L lipid titer from CO₂ (FIG. 6C). When a comparable load (4.6±0).7 g/L) of fads was inoculated, the lipid titer from CO₂ increased to 1194.8±165.2 mg/L (FIG. 6C), demonstrating the better C2 utilization and lipid productivity of fads in the system. When the Cu₆Zn₁ catalyst is used, the fads strain (4.8±0.1 g/L cell density) increases the lipid titer to 1840.2±3.3 mg/L (FIG. 6C), indicating the effectiveness of the co-design of strain and catalysts. Notably, aside from C2 substrates, formate and propanol are also present in the CO₂RR products, in which formate could be catabolized to generate reducing equivalents to drive bioconversion, and propanol serves as carbon source to support both cell growth and lipid production of RHA1 (FIGS. 24 and 25). Overall, the lipid productivity of the integrated system with the Cu₆Zn₁+fads combination can produce 1840.2+3.3 mg/L/day of lipids, which is about six-fold of the highest lipid productivity achieved by algae (Chlorella sp. HS2, 289.6 mg/L/day) through photosynthesis (FIG. 6D)⁴⁴⁻⁴⁶, highlighting that the electro-biodiesel system and co-design of catalyst and microorganism can produce lipid from CO₂ faster than the nature systems.

To evaluate the energy conversion efficiency of the integrated electro-biodiesel system, we decomposed the conversion into three stages: solar to electricity, electricity to soluble carbon sources, and soluble carbon sources to lipid. For the stage I conversion, we estimated the efficiency using maturing photovoltaic technology, indicating an energy efficiency of 25% (FIG. 6E).47 The energy efficiency of the second stage electrocatalysis was calculated based on enthalpy gains for specific CO₂RR products.⁴⁸ Considering the gaseous products such as ethylene and hydrogen from electrocatalysis were not utilized by the bioconversion, we calculated the energy efficiency by dividing the energy content of all soluble products (energy output) with the electricity energy input used to produce these soluble products (see Methods).^{15,48} The Cu₆Zn₁ catalyst reached an energy efficiency of 49.2±5.6% for the conversion of electricity into soluble products (FIG. 6E). Third, the energy efficiency of bioconversion is calculated by dividing the energy content of the produced lipid by the total energy of consumed soluble products, resulting in an average efficiency of 36.5±1.5% for the fads strain in the integrated system (FIG. 6E). Fourth, the energy efficiency of transesterification is calculated by dividing the energy content of theoretically obtained fatty acid methyl ester by the energy of 1 kg of lipids and the required methanol, then multiplying by the experimentally determined reaction yield of 98.8±0.5%, resulting in an efficiency of 96.0±0.2%. Overall, the electro-biodiesel system achieves an overall energy efficiency of 4.5% for converting CO₂ to lipids, and 4.3% for converting CO₂ to biodiesel (FIG. 6E). This result approaches the theoretical maximum achievable efficiency of 5.5%, which is calculated by combining the current highest energy efficiency of CO₂ electroreduction for C2 production with the highest energy efficiency observed in microbial fermentation using C2 substrates (FIG. 1).

The solar-to-molecule efficiency of 4.5% by this study surpassed competitive platforms such as soybean biodiesel and algal biodiesel and exceeded the solar-to-biomass efficiency achieved in recent studies (see Methods). The remarkable efficiency indicates that one acre of land can theoretically yield 875,880 MJ of electro-biodiesel annually, approximately forty-eight times higher than soybean biodiesel, and three times of the achievable algal biodiesel energy production (Table 2, FIG. 1). A major advantage of electro-biodiesel over phototrophic and mixotrophic algal cultivation is overcoming the dilemma of mutual shading at high cell density in algal cultivation⁴. Our previous study has highlighted that light penetration began to limit cell growth at OD about 2 in cyanobacteria, when mutual shading will block the light and limit the growth. However, in our electro-bio integrated system, we operated at OD 4.5 to 5 to achieve high productivity and titer. Even higher productivity can be achieved when high-density cell cultivation is used. High productivity and titer are critical as they will drive better economics and efficiency. Compared with the plant and algal based biodiesel, where solar-to-molecule efficiency is limited by both photosynthesis efficiency and low carbon partition into energy-dense storage carbon for lipid production, resulting in low per-acre yields, the electro-biodiesel overcomes the low efficiency, kinetics, and carbon partition through both electrocatalytic CO₂RR to produce biocompatible high energy content electron carrier and engineering of energy and reductant balance in a highly efficient oleaginous bacteria. As compared to the previous platforms focusing on acetate production from CO and CO₂ 49.50, the mechanism-driven synthetic biology engineering and unique co-design of microorganisms and catalyst empowered the optimal composition of C2 intermediates and, consequently, highly efficient conversion. In particular, the reductant and ATP from ethanol assimilation can improve the bioenergetic and metabolic efficiency of acetate conversion. The overall bioconversion efficiency for the C2 intermediate mixture to the highly reduced lipid molecule can achieve 37%. The fundamental discoveries and the engineering designs opened new avenues for substantially improving energy and carbon conversion efficiency in both microbes and the integrated electro-bio system, providing path for efficient electron-to-molecule conversion. The high energy conversion efficiency and lipid productivity also translate into less land usage for electro-biodiesel production. Specifically, the electro-biodiesel production requires only ⅓ of the land required algal biodiesel, 1/45 of the land required by soybean biodiesel, and 1/10 of the land required by palm biodiesel, amounting to only 0.83% of land to sustain the entire U.S. diesel consumption (Table 2).

The life cycle analysis (LCA) was conducted to evaluate the global warming impact of the electro-biodiesel approach, considering the three primary processes: electrolysis, fermentation, and lipid extraction. The functional unit, system boundaries, and inventory analysis were provided in FIG. 26 and Table 4. The electricity utilization for CO₂ electroreduction, and microorganism culturing are the primary contributors to the CO₂ emissions (CO₂e) from electro-biodiesel life cycle (FIG. 27). Scenario analysis further examined the climate impact of different electricity sources and byproduct allocation, identifying the former as the primary factor to affect the carbon emission. By substituting conventional electricity with renewable sources, the electro-biodiesel approach could achieve a reduction of 1.57 g of CO₂ per gram of electro-biodiesels produced together with the by-products such as biomass, ethylene, and others (FIG. 27, Tables 5 and 6). Both lipid and by-products are of commercial value and contribute to the emission reduction by electro-biodiesel.

The results highlight the potential for electro-biodiesel to achieve negative emission, in contrast to diesel produced from petroleum fractionation (0.52 g CO₂e/g) and other biodiesel production methods, which typically have positive CO₂ emission ranging from 2.5 to 9.9 g CO₂e/g per gram of lipids produced (Table 7).⁵¹

To assess the economic performance of an scaled up electro-biodiesel system, a techno-economic analysis (TEA) was performed utilizing the experimental data and an annual output of approximately 8,000 tons, which was used in a previous TEA analysis on heterotrophic microbial lipid production process.⁵² The results for the LCA case of renewable energy source with byproduct offset credit are integrated into the TEA (FIG. 28).⁵³ Our base case analysis revealed a minimum lipid selling price at $2.36/kg with the current electro-biodiesel system's performance and the assumed production capacity (FIG. 6F). This selling price outcompeted the previous estimate of $2.5/kg for microbial lipid production at the same annual scale using glucose as feedstock.⁵² This estimated price also demonstrates that electro-biodiesel approach is cost competitiveness to microalgae lipid production in both photobioreactors ($20.53/gal) and open pounds ($9.84/gal).54 It highlighted the superior performance of the electro-biodiesel approach as compared to the traditional biorefinery-based biofuel and photosynthetic biofuel approaches. 2 The results of sensitivity analysis are shown in FIG. 14 which identifies several key parameters that would further impact the minimum lipid selling price. It has the potential for electro-biodiesel prices to compete with plant oil prices, which range from $0.5 to $1.9/kg.⁵⁵

We have taken a systemic approach to design the electro-biodiesel route, identify the fundamental limits, and improve the system efficiency, economics, and emission impacts. The new route leverages the high efficiency of electrocatalysis and synthesis of long chain fuels from microorganisms. In order to achieve high system efficiency and kinetics, we first investigated the biochemical and metabolic limits for C2+ intermediate conversion and found that ethanol conversion is unexpectedly low in experimental data as compared to the modeling. We then identified the acidification stress, reducing equivalent imbalance, and low ATP production as the metabolic limits to prevent efficient ethanol conversion into lipid in a model oleaginous microorganism R. jostii RHA1. To overcome the metabolic limits, metabolic engineering was carried out to mitigate cellular acidification, balance reductant generation, and increase ATP production, all of which has enhanced carbon flux to lipids using C2 intermediates. Using the engineered strain, we further explored the co-substrate synergy for acetate and ethanol, considering that ethanol conversion in the engineered strain could render more reducing equivalents and higher ATP to drive carbon conversion. The study revealed the synergistic effects and identified proper ratio for ethanol and acetate to achieve maximized lipid conversion. Based on co-substrate effect, we designed a new Zn—Cu bimetallic catalyst that efficiently produces C2 intermediates in biocompatible electrolyte at an optimal acetate/ethanol ratio for lipid synthesis in engineered RHA1 strain. The co-design of Zn—Cu catalyst and RHA1 strain enabled a highly efficient electro-microbial integrated system to achieve 1840.2±3.3 mg/L/day lipid productivity from CO₂ with a 4.3% solar-to-fuel overall energy efficiency, significantly surpassing the photosynthesis-based biofuel production strategy. Additionally, the utilization of C2 intermediates in electro-biodiesel route imparts the versatility to allow for the incorporation of various microorganisms to achieve diverse fuel chemical production. Moreover, the electro-biodiesel can achieve substantial emission impact reduction and at-1.57 g CO₂/g electro-biodiesel produced, and a market competitive price under large-scale production is US$2.36/kg lipids. This study thus establishes a highly productive, efficient, cost-friendly, and carbon-negative electro-biodiesel route that directly uses CO₂ as feedstock to fulfill all the U.S. diesel demands with less than 1% of land. Such land usage does not have to be arable lands, thus substantially alleviates food-energy competition and the shortage of biodiesel feedstock. The study further proves the concept for a broad platform for highly efficient conversion of renewable energy into chemicals, fuels, and materials to address the fundamental limits of human civilization.

TABLE 2
Comparison between electro-biodiesel and existing biodiesel production platforms.

| | Electro- | | Algal | | Soybean | | Palm | |
|---|---|---|---|---|---|---|---|---|
| Platforms | biodiesel | | biodiesel | | biodiesel | | biodiesel | |
| Global biodiesel | 3,003,216 × | | 3,003,216 × | | 3,003,216 × | | 3,003,216 × | |
| consumption^{§} | 10⁶ | | 10⁶ | | 10⁶ | | 10⁶ | |
| (MJ/year) | | | | | | | | |
| U.S. diesel | 8,276,728.32 × | | 8,276,728.32 × | | 8,276,728.32 × | | 8,276,728.32 × | |
| consumption | 10⁶ | | 10⁶ | | 10⁶ | | 10⁶ | |
| (MJ/year) | | | | | | | | |
| Photovoltaic energy | 2,913,739 | | N/A | | N/A | | N/A | |
| output | | | | | | | | |
| (MJ/acre/year) | | | | | | | | |
| EE_{electricity-to-biodiesel}^{¶} | 17.2% | | N/A | | N/A | | N/A | |
| Energy yield* | 501,163 | | 175,104 | | 11,075.3 | | 49,481 | |
| (MJ/acre/year) | | | | | | | | |
| Land use^{#} | 5.99 | 16.5 | 17.2 | 47.3 | 271 | 747 | 60.7 | 167.3 |
| (million acres) | | | | | | | | |
| Land use | 0.30% | 0.83% | 0.86% | 2.4% | 13.6% | 37% | 3.0% | 8.4% |
| percentage^{†} | | | | | | | | |

^{§}Worldwide biodiesel consumption is about 65.86 million tons in 2023, equivalent to 3,003,216 × 10⁶ MJ per year.⁴
^{Δ} U.S. biodiesel consumption is about 3.7 million barrels per day, which is equivalent to 8,276,728.32 × 10⁶ MJ per year.
^{¶}Energy efficiency (EE) of electricity-to-diesel of the electro-biodiesel system, calculated by multiplying EE of electricity-to-C2, EE of C2-to-lipid, and EE of lipid-to-biodiesel (See Calculation of energy efficiency for the Electro-biodiesel system).
*Values show energy production rate per year per unit area in MJ (See Land use evaluation of different biodiesel platforms).
^{#}Values denote the theoretical land acres required to sustain the global biodiesel consumption (left column) or U.S. annual diesel consumption (right column). The values are calculated by dividing the energy of global biodiesel consumption or U.S. annual diesel consumption by the energy yield of each diesel production platform.
^{†}Values are calculated by dividing the land use for each diesel production platforms by the total land area of the continental 48 states of the U.S. which is 1,996.7 million acers.

TABLE 3
Inputs and outputs data per 1 gram of lipids

| Inputs | Usage | unit |
|---|---|---|
| CO₂ | 422.70 | g |
| Culture Medium (Table 4) | 1 | L |
| Electricity | 4.63 | kWh |
| Outputs | Production | unit |
| Lipids | 1 | g |
| H₂ | 0.26 | g |
| CO | 3.19 | g |
| CH₄ | 0.02 | g |
| C₂H₄ | 2.92 | g |
| biomass | 0.76 | g |
| CO₂ (recycled) | 398.69 | g |
| CO₂ (direct) | 5.54 | g |

TABLE 4
Elements of 1 L Culture Medium and Associated CO₂e Emissions

| | | | | Emission factors | Total CO₂e |
|---|---|---|---|---|---|
| | | | Usage | (g CO₂e/g | Emissions |
| Culture | Concentration | Unit | (g) | product) | (g) |
| KH₂PO₄ | 1.70 | g/L | 1.70E+00 | 3.00 | 5.10 |
| Na₂HPO₄ | 9.80 | g/l | 9.80E+00 | 0.31 | 3.04 |
| MgSO₄ | 0.10 | mg/l | 1.00E−04 | 0.30 | 0.00 |
| FeSO₄•7H₂O | 0.95 | mg/l | 9.50E−04 | 0.18 | 0.00 |
| MgO | 10.75 | mg/l | 1.08E−02 | 0.71 | 0.01 |
| CaCO₃ | 2.00 | mg/l | 2.00E−03 | 0.01 | 0.00 |
| ZnSO₄ | 1.20 | mg/l | 1.20E−03 | 0.82 | 0.00 |
| CuSO₄ | 0.20 | mg/l | 2.00E−04 | 1.00 | 0.00 |
| CoSO₄•7H₂O | 0.15 | mg/l | 1.50E−04 | 6.36 | 0.00 |
| H₃BO₄ | 0.06 | mg/l | 6.00E−05 | 0.72 | 0.00 |
| HCl | 51.30 | ml/l | 5.13E−02 | 1.20 | 0.06 |
| | | | | Total | 8.21 |

Note:
The emission factors are sourced from the following websites: For KH₂PO₄⁶ and CaCO₃⁷ are both sourced from CarboClound. The emission factors for the rest chemicals are sourced from Winnipeg.⁸

TABLE 5
Life Cycle Impact Assessment of electro-biodiesel system

| | | Greenhouse Gas Emissions (g CO₂e/g lipid) | | | |
|---|---|---|---|---|---|
| | | Scenario | Scenario | Scenario | Scenario |
| Produce 1 g lipid | | 1 | 2 | 3 | 4 |
| CO₂ Electrolysis | Intake CO₂ | −422.7 | −422.7 | −422.7 | −422.7 |
| | Direct CO2 loss | 5.54 | 5.54 | 5.54 | 5.54 |
| | CO₂ in System | 398.69 | 398.69 | 398.69 | 398.69 |
| | Recycle | | | | |
| Fermentation | Culture (details in | 8.21 | 8.21 | 8.21 | 8.21 |
| | Table 4) | | | | |
| Extraction and | Chloroform for | 3.7 | 3.7 | 3.7 | 3.7 |
| centrifugation | extraction | | | | |
| Transesterification | Methanol-HCl and | 1.29 | 1.29 | 1.29 | 1.29 |
| | toluene for | | | | |
| | Transesterification | | | | |
| Electricity Total | CO₂ electrolysis | 188.03 | 188.03 | ~0 | ~0 |
| | PSA | 2.65 | 2.65 | ~0 | ~0 |
| | Fermentation | 114.47 | 114.47 | ~0 | ~0 |
| | Extraction and | 47.3 | 47.3 | ~0 | ~0 |
| | Decenter | | | | |

| | | | | |
|---|---|---|---|---|
| Byproducts | 3.70 | −5.75 | 3.70 | −5.75 |
| Total Greenhouse Gas Emissions | 349.59 | 343.85 | −2.86 | −8.61 |
| (g CO₂e/g lipid) | | | | |
| Total Greenhouse Gas Emissions | 350.88 | 345.14 | −1.57 | −7.32 |
| (g CO₂e/g biodiesel) | | | | |

Note:
the emission factor of chloroform is 4.13 g CO₂e/g chloroform from GREET.⁹ According to the current studies, chloroform could be recycled, and 2% loss rate is assumed.¹⁰ Scenarios 1 and 2 assumed the system used electricity from NGCC power plants with CCS equipment. Scenarios 3 and 4 assume the system is powered by renewable electricity without extra GHG emissions. Scenarios 2 and 4 made an additional assumption that the byproducts from the system can displace conventional products and offset the GHG emissions from conventional sources. The sensitivity analysis is based on the following assumptions: 1) CO₂ from bacterial culturing and lipid fermentation can be recycled for the CO₂ electroreduction; 2) Soluble C2 products that are not completely utilized to produce 1 gram lipid can be recycled and utilized by next round; 3) The amount of salts and trace elements in the medium for bacterial culturing and lipid fermentation is not recycled.

TABLE 6
Byproduct Allocation

| | | Emission |
|---|---|---|
| Byproduct (1 g) | Alternative Source | Factors |
| Biomass | 1 g biomass from corn stover | 0.06 |
| H₂ | 1 g hydrogen from renewable natural gas | 0.64 |
| C₂H₄ | 1 g ethylene from corn stover | 0.63 |
| CO | 1 g carbon monoxide from polyethylene | 0.97 |
| | terephthalate resin | |
| CH₄ | 1 g methane is equivalent to 28 g CO₂ for | 28 |
| | 100-year global warming potential | |

Note:
The emission factor of CH₄ is referenced from the web site source¹¹. The rest emission factors are referenced from GREET.⁹

TABLE 7
Comparison of carbon emissions of
different diesel production routes.

| | | CO2e Emissions | | |
|---|---|---|---|---|
| Production Route | Feedstock | (g/g) | | Reference |
| Fractionation | Petroleum | 0.52 | | GREET |
| Transesterification | Soybean | 0.73 | | GREET |
| Transesterification | Soybean/canola/ | 21-31 | g/MJ | ¹² |
| | carinata | | | |
| Hydro-processing | Soybean/canola/ | 22.68-34.1 | g/MJ | ¹² |
| | carinata | | | |

| | | | |
|---|---|---|---|
| Transesterification | Microalgae | 0.85-1.46 | ¹³ |
| Lipid only | Microalgae | 2.5-9.9 | ¹⁴ |

TABLE 8
Plasmids and strains used in this study

| Plasmids and strains | Genotype or description | Reference |
|---|---|---|
| Plasmids | | |
| PDD120 | pConstitutive-Che9c60::Che9c61: pBR322 origin | ¹⁵ |
| | of replication, km^{R}, Gene-overexpression plasmid. | |
| PBSNC9031-Pben- | pBenA-FASI: pNC903 origin of replication, Ts^{R}, | ¹⁶ |
| FAS | E. coli-Rhodococcus shuttle plasmid for fasI and | |
| | atf2 overexpression in R. opacus. | |
| PDD120-dmpF- | pConstitutive-dmpF::sthA: pBR322 origin of | This study |
| ArtRBS-sthA | replication, km^{R}, overexpression dmpF and sthA. | |
| PBSNC9031-fasI- | pBenA-FASI::pConstitutive-dmpF::sthA: pNC903 | This study |
| atf2-dmpF-sthA | origin of replication, Ts^{R}, E. coli-Rhodococcus | |
| | shuttle plasmid for overexpression of fasI, atf2, | |
| | dmpF, and sthA in R. jostii RHA1. | |
| Strains | | |
| E. coli K-12 DH 5α | K12 derivative; F⁻, λ-, hsdR(rk⁻, mk⁻), supE44, thi- | ¹⁷ |
| | 1, recAl, endAl, Δ(lacZYA-argF)U169, | |
| | Φ80dlacZ, ΔM15, deoR, nupG | |
| R. jostii RHA1 | Wild type | ¹⁸ |
| dmpF-sthA | dmpF and sthA overexpression | This study |
| fasI-atf2 | fasI and atf2 overexpression | This study |
| fasI-atf2-dmpF-sthA | fasI, atf2, dmpF and sthA overexpression | This study |
| (fads) | | |

TABLE 9
Primers used in this study

| Primers | Sequence | Purpose |
|---|---|---|
| sthA_F | ATGCCACATTCCTACGATTA (SEQ ID | Amplify sthA for |
| | NO: 1) | overlapping PCR |
| sthA_R | TTAAAACAGGCGGTTTAAA (SEQ ID | Amplify sthA for |
| | NO: 2 | overlapping PCR |
| ArtRBS-sthA_F | CCTCTGTTCCCAAGGGGAGAAGCCG | Amplify sthA and add rbs |
| | AAACATAAAAAGGAGGTCTTTTATGC | for overlapping PCR |
| | CACATTCCTACGATTA (SEQ ID NO: 3) | |
| ArtRBS-sthA_R | TTAAAACAGGCGGTTTAAA (SEQ ID | Amplify sthA and add rbs |
| | NO: 4) | for overlapping PCR |
| G2-dmpF_F1 | AttaagaaggagatatacatATGACCAAGGCAA | Amplify dmpF for |
| | GTGTGGC (SEQ ID NO: 5) | overlapping PCR. Gibson |
| | | assemble for PDD120- |
| | | dmpF-sthA |
| G2-dmpF_R1 | TCTCCCCTTGGGAACAGAGGTCATGC | Amplify dmpF for |
| | CTCCACGCTCAGGAG (SEQ ID NO: 6) | overlapping PCR. |
| | | Gibson assemble for |
| | | PDD120-dmpF-sthA |
| G3_F2 | TCCTGAGCGTGGAGGCATGACCTCTG | Gibson assemble for |
| | TTCCCAAGGGGAG (SEQ ID NO: 7) | PDD120-dmpF-sthA |
| G3_R2 | cgttgtacttttcggccttctcaaaaaagccggttcaggcc | Gibson assemble for |
| | (SEQ ID NO: 8) | PDD120-dmpF-sthA |
| G4_Fvec | gcctgaaccggcttttttgagaaggccgaaaagtacaacgac | Gibson assemble for |
| | (SEQ ID NO: 9) | PDD120-dmpF-sthA |
| G4_Rvec | GCCACACTTGCCTTGGTCATatgtatatctcc | Gibson assemble for |
| | ttcttaattaagcatgcgga (SEQ ID NO: 10) | PDD120-dmpF-sthA |
| PbenA-fasI_F | TACTCCGGGTACCTGTGCGG (SEQ ID | amplify fasI for |
| | NO: 11) | overlapping |
| PbenA-fasI_R | CTACTTGCAGCCGGGCAGACCC (SEQ | Amplify fasI for |
| | ID NO: 12) | overlapping |
| rest_F | GAACGACGGATGGGAGTTCTGG (SEQ | amplify PNSNC9031 |
| | ID NO: 13) | vector with atf2 gene for |
| | | overlapping |
| rest_R | ACATGTGAGCAAAAGGCCAGCAAAA | amplify PNSNC9031 |
| | GG (SEQ ID NO: 14) | vector with atf2 gene for |
| | | overlapping |
| pC-dmpF_F | TGTGCGGGCTCTAAC (SEQ ID NO: 15) | Amplify pConstitutive- |
| | | dmpF-ArtRBS-sthA for |
| | | overlapping |
| sthA_R | TTAAAACAGGCGGTTTAAA (SEQ ID | Amplify pConstitutive- |
| | NO: 16) | dmpF-ArtRBS-sthA for |
| | | overlapping |
| GibInte_fasI_F1 | CTGGCCTTTTGCTCACATGTTACTCCG | Gibson assemble for |
| | GGTACCTGTGC (SEQ ID NO: 17) | PBSNC9031-fasI-atf2- |
| | | dmpF-sthA |
| GibInte_fasI_R1 | gacgtgttagagcccgcacaCTACTTGCAGCCG | Gibson assemble for |
| | GG (SEQ ID NO: 18) | PBSNC9031-fasI-atf2- |
| | | dmpF-sthA |
| GibInte_pC-dmpF_F2 | GTCTGCCCGGCTGCAAGTAGtgtgcgggct | Gibson assemble for |
| | ctaac (SEQ ID NO: 19) | PBSNC9031-fasI-atf2- |
| | | dmpF-sthA |
| GibInte_pC-dmpF_R2 | AGAACTCCCATCCGTCGTTCTTAAAA | Gibson assemble for |
| | CAGGCGGTTTAAA (SEQ ID NO: 20) | PBSNC9031-fasI-atf2- |
| | | dmpF-sthA |
| GibInte_vec_F | TTTAAACCGCCTGTTTTAAGAACGAC | Gibson assemble for |
| | GGATGGGAGTTCT (SEQ ID NO: 21) | PBSNC9031-fasI-atf2- |
| | | dmpF-sthA |
| GibInte_vec_R | CCGCACAGGTACCCGGAGTAACATGT | Gibson assemble for |
| | GAGCAAAAGGCCAGCA (SEQ ID NO: | PBSNC9031-fasI-atf2- |
| | 22) | dmpF-sthA |

### SUPPLEMENTAL REFERENCES FOR FIGS. 7-29 AND TABLES 2-9

1 Xiao, Y., Ruan, Z., Liu, Z., Wu, S. G., Varman, A. M., Liu, Y. and Tang, Y. J. (2013). Engineering Escherichia coli to convert acetic acid to free fatty acids. Biochemical Engineering Journal 76, 60-69.
2 Pang, Z., Chong, J., Zhou, G., de Lima Morais, D. A., Chang, L., Barrette, M., Gauthier, C., Jacques, P.-É., Li, S. and Xia, J. (2021). MetaboAnalyst 5.0: narrowing the gap between raw spectra and functional insights. Nucleic Acids Research 49, W388-W396.
3 SRI International. Pathway Collages. https://metacyc.org/pathway-collage-info.
4 Statista. Biodiesel consumption worldwide from 2004 to 2023, with a forecast until 2030. https://www.statista.com/statistics/1440983/worldwide-consumption-of-biodiesel/#:˜:text=In % 202023%2C %20worldwide %20biodiesel %20consumption,mil lion % 20metric %20tons %20 in %202030.
5 Arkin, A. P., Cottingham, R. W., Henry, C. S., Harris, N. L., Stevens, R. L., Maslov, S., Dehal, P., Ware, D., Perez, F., Canon, S., Sneddon, M. W., Henderson, M. L., Riehl, W. J., Murphy-Olson, D., Chan, S. Y., Kamimura, R. T., Kumari, S., Drake, M. M., Brettin, T. S., Glass, E. M., Chivian, D., Gunter, D., Weston, D. J., Allen, B. H., Baumohl, J., Best, A. A., Bowen, B., Brenner, S. E., Bun, C. C., Chandonia, J.-M., Chia, J.-M., Colasanti, R., Conrad, N., Davis, J. J., Davison, B. H., DeJongh, M., Devoid, S., Dietrich, E., Dubchak, I., Edirisinghe, J. N., Fang, G., Faria, J. P., Frybarger, P. M., Gerlach, W., Gerstein, M., Greiner, A., Gurtowski, J., Haun, H. L., He, F., Jain, R., Joachimiak, M. P., Keegan, K. P., Kondo, S., Kumar, V., Land, M. L., Meyer, F., Mills, M., Novichkov, P. S., Oh, T., Olsen, G. J., Olson, R., Parrello, B., Pasternak, S., Pearson, E., Poon, S. S., Price, G. A., Ramakrishnan, S., Ranjan, P., Ronald, P. C., Schatz, M. C., Seaver, S. M. D., Shukla, M., Sutormin, R. A., Syed, M. H., Thomason, J., Tintle, N. L., Wang, D., Xia, F., Yoo, H., Yoo, S. and Yu, D. (2018). KBase: The United States Department of Energy Systems Biology Knowledgebase. Nature Biotechnology 36, 566-569.
6 CarbonCloud. Potassium phosphate E340. https://apps.carboncloud.com/climatehub/product-reports/id/71624889280.
7 CarbonCloud. Limestone (CaCO3). https://apps.carboncloud.com/climatehub/product-reports/id/43838269287.
8 Winnipeg. Emission factors in kg CO₂-equivalent per unit. https://legacy.winnipeg.ca/finance/findata/matmgt/documents/2012/682-2012/682-2012_appendix_h-wstp_south_end_plant_process_selection_report/appendix % 207.pdf.
9 Center for Transportation Research Energy Systems Division. Argonne GREET Model. https://greet.anl.gov/.
10 Vea, E. B., Fabbri, S., Spierling, S. and Owsianiak, M. (2021). Inclusion of multiple climate tipping as a new impact category in life cycle assessment of polyhydroxyalkanoate (PHA)-based plastics. Science of The Total Environment 788, 147544.
11 Intergovernmental Panel on Climate Change. CLIMATE CHANGE 2023 Synthesis Report. https://www.ipcc.ch/report/ar6/syr/downloads/report/IPCC_AR6_SYR_SPM.pdf.
12 Xu, H., Ou, L., Li, Y., Hawkins, T. R. and Wang, M. (2022). Life Cycle Greenhouse Gas Emissions of Biodiesel and Renewable Diesel Production in the United States. Environmental Science & Technology 56, 7512-7521.
13 Saranya, G. and Ramachandra, T. V. (2020). Life cycle assessment of biodiesel from estuarine microalgae. Energy Conversion and Management: X 8, 100065.
14 Parsons, S., Allen, M. J., Abeln, F., McManus, M. and Chuck, C. J. (2019). Sustainability and life cycle assessment (LCA) of macroalgae-derived single cell oils. Journal of Cleaner Production 232, 1272-1281.
15 DeLorenzo, D. M., Rottinghaus, A. G., Henson, W. R. and Moon, T. S. (2018). Molecular Toolkit for Gene Expression Control and Genome Modification in Rhodococcus opacus PD630. ACS Synth Biol 7, 727-738.
16 Xie, S., Sun, S., Lin, F., Li. M., Pu, Y., Cheng, Y., Xu, B., Liu, Z., da Costa Sousa, L., Dale, B. E., Ragauskas, A. J., Dai, S. Y. and Yuan, J. S. (2019). Mechanism-Guided Design of Highly Efficient Protein Secretion and Lipid Conversion for Biomanufacturing and Biorefining. Advance Science (Weinh) 6, 1801980.
17 Soundararajan, M., von Bunau, R. and Oelschlaeger, T. A. (2019). K5 Capsule and Lipopolysaccharide Are Important in Resistance to T4 Phage Attack in Probiotic E. coli Strain Nissle 1917. Frontier in Microbiology 10, 2783.
18 McLeod, M. P., Warren, R. L., Hsiao, W. W. L., Araki, N., Myhre, M., Fernandes, C., Miyazawa, D., Wong, W., Lillquist, A. L., Wang, D., Dosanjh, M., Hara, H., Petrescu, A., Morin, R. D., Yang, G., Stott, J. M., Schein, J. E., Shin, H., Smailus, D., Siddiqui, A. S., Marra, M. A., Jones, S. J. M., Holt, R., Brinkman, F. S. L., Miyauchi, K., Fukuda, M., Davies, J. E., Mohn, W. W. and Eltis, L. D. (2006). The complete genome of Rhodococcus sp. RHA1 provides insights into a catabolic powerhouse. Proceedings of the National Academy of Sciences 103, 15582-15587.
19 Ooms, M. D., Dinh, C. T., Sargent, E. H. and Sinton, D. (2016). Photon management for augmented photosynthesis. Nature Communication 7, 12699.
20 Blankenship, R. E., Tiede, D. M., Barber, J., Brudvig, G. W., Fleming, G., Ghirardi, M., Gunner, M. R., Junge, W., Kramer, D. M., Melis, A., Moore, T. A., Moser, C. C., Nocera, D. G., Nozik, A. J., Ort, D. R., Parson, W. W., Prince, R. C. and Sayre, R. T. (2011). Comparing Photosynthetic and Photovoltaic Efficiencies and Recognizing the Potential for Improvement. Science 332, 805-809.
21 Ahmad, L., Khordehgah, N., Malinauskaite, J. and Jouhara, H. (2020). Recent advances and applications of solar photovoltaics and thermal technologies. Energy 207.
22 Zhang, G., Wang, T., Zhang, M., Li, L., Cheng, D., Zhen, S., Wang, Y., Qin, J., Zhao, Z.-J. and Gong, J. (2022). Selective CO₂ electroreduction to methanol via enhanced oxygen bonding. Nature Communications 13, 7768.
23 Obasanjo, C. A., Gao, G., Crane, J., Golovanova, V., García de Arquer, F. P. and Dinh, C.-T. (2023). High-rate and selective conversion of CO₂ from aqueous solutions to hydrocarbons. Nature Communications 14, 3176.
24 Wang, P., Yang, H., Tang, C., Wu, Y., Zheng, Y., Cheng, T., Davey, K., Huang, X. and Qiao, S.-Z. (2022). Boosting electrocatalytic CO₂-to-ethanol production via asymmetric C-C coupling. Nature Communications 13, 3754.
25 Hann, E. C., Overa, S., Harland-Dunaway, M., Narvaez, A. F., Le, D. N., Orozco-Cárdenas, M. L., Jiao, F. and Jinkerson, R. E. (2022). A hybrid inorganic-biological artificial photosynthesis system for energy-efficient food production. Nature Food 3, 461-471.
26 Claassens, N. J., Cotton, C. A. R., Kopljar, D. and Bar-Even, A. (2019). Making quantitative sense of electromicrobial production. Nature Catalysis 2, 437-447.
27 U.S. Energy Information Administration. SHORT-TERM ENERGY OUTLOOK. https://www.eia.gov/outlooks/steo/report/BTL/2023/06-dieselmacro/article.php.
28 National Renewable Energy Laboratory. How much land will PV need to supply our electricity? https://www.nrel.gov/docs/fy04osti/35097.pdf.
29 National Renewable Energy Laboratory. The Potential for Biofuels from Algae. https://www.nrel.gov/docs/fy08osti/42414.pdf.
30 United States Department of Agriculture. Crop Production 2023 Summary. https://downloads.usda.library.cornell.edu/usda-esmis/files/k3569432s/ns065v292/8910md644/cropan24.pdf.
31 USDA Foreign Agricultural Service. Production Palm Oil. https://fas.usda.gov/data/production/commodity/4243000
32 Our World in Data. Palm Oil. https://ourworldindata.org/palm-oil.
33 USDA Agricultural Service. (2023). Biofuels Annual. chrome-extension://efaidnbmnnnibpcajpcglclefindmkaj/https://apps.fas.usda.gov/newgainapi/api/Report/DownloadReportBy FileName?fileName=Biofuels % 20Annual_Kuala %20 Lumpur_Malaysia_MY2023-0012.pdf
34 Alvarez, H. M., Herrero, O. M., Silva, R. A., Hernandez, M. A., Lanfranconi, M. P. and Villalba, M. S. (2019). Insights into the Metabolism of Oleaginous Rhodococcus spp. Applied and Environmental Microbiology 85, e00498-00419.
35 Niemann, B., Haufs-Brusberg, S., Puetz, L., Feickert, M., Jaeckstein, M. Y., Hoffmann, A., Zurkovic, J., Heine, M., Trautmann, E.-M., Müller, C. E., Tönjes, A., Schlein, C., Jafari, A., Eltzschig, H. K., Gnad, T., Blüher, M., Krahmer, N., Kovacs, P., Heeren, J. and Pfeifer, A. (2022). Apoptotic brown adipocytes enhance energy expenditure via extracellular inosine. Nature 609, 361-368.
36 Wang, X., Liu, W., Xin, C., Zheng, Y., Cheng, Y., Sun, S., Li, R., Zhu, X.-G., Dai, S. Y., Rentzepis, P. M. and Yuan, J. S. (2016). Enhanced limonene production in cyanobacteria reveals photosynthesis limitations. Proceedings of the National Academy of Sciences 113, 14225-14230.
37 Fiehn, O. and Kind, T. in Metabolomics: Methods and Protocols (ed Wolfram Weckwerth) 3-17 (Humana Press, 2007).
38 Fiehn, O., Wohlgemuth, G., Scholz, M., Kind, T., Lee, D. Y., Lu, Y., Moon, S. and Nikolau, B. (2008). Quality control for plant metabolomics: reporting MSI-compliant studies. The Plant Journal 53, 691-704.
39 Matyash, V., Liebisch, G., Kurzchalia, T. V., Shevchenko, A. and Schwudke, D. (2008). Lipid extraction by methyl-tert-butyl ether for high-throughput lipidomics. Journal of Lipid Research 49, 1137-1146.
40 Tsugawa, H., Cajka, T., Kind, T., Ma, Y., Higgins, B., Ikeda, K., Kanazawa, M., VanderGheynst, J., Fiehn, O. and Arita, M. (2015). MS-DIAL: data-independent MS/MS deconvolution for comprehensive metabolome analysis. Nature Methods 12, 523-526.
41 DeFelice, B. C., Mehta, S. S., Samra, S., Čajka, T., Wancewicz, B., Fahrmann, J. F. and Fiehn, O. (2017). Mass Spectral Feature List Optimizer (MS-FLO): A Tool To Minimize False Positive Peak Reports in Untargeted Liquid Chromatography-Mass Spectroscopy (LC-MS) Data Processing. Analytical Chemistry 89, 3250-3255.
42 Kind, T., Liu, K.-H., Lee, D. Y., DeFelice, B., Meissen, J. K. and Fiehn, O. (2013). LipidBlast in silico tandem mass spectrometry database for lipid identification. Nature Methods 10, 755-758.
43 Zhang, P., Chen, K., Xu, B., Li, J., Hu, C., Yuan, J. S. and Dai, S. Y. (2022). Chem-Bio interface design for rapid conversion of CO₂ to bioplastics in an integrated system. Chem 8, 3363-3381.
44 Liu, C., Colón, B. C., Ziesack, M., Silver, P. A. and Nocera, D. G. (2016). Water splitting-biosynthetic system with CO₂ reduction efficiencies exceeding photosynthesis. Science 352, 1210-1213.
45 Khoo, K. S., Ahmad, I., Chew, K. W., Iwamoto, K., Bhatnagar, A. and Show, P. L. (2023). Enhanced microalgal lipid production for biofuel using different strategies including genetic modification of microalgae: A review. Progress in Energy and Combustion Science 96, 101071.
46 Hossain, F. M., Rainey, T. J., Ristovski, Z. and Brown, R. J. (2018). Performance and exhaust emissions of diesel engines using microalgae FAME and the prospects for microalgae HTL biocrude. Renewable and Sustainable Energy Reviews 82, 4269-4278.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated within the scope of the invention without limitation thereto.

### REFERENCES

1 Mertens, J., Breyer, C., Arning, K., Bardow, A., Belmans, R., Dibenedetto, A., Erkman, S., Gripekoven, J., Leonard, G., Nizou, S., Pant, D., Reis-Machado, A. S., Styring, P., Vente, J., Webber, M. and Sapart, C. J. (2023). Carbon capture and utilization: More than hiding CO₂ for some time. Joule 7, 442-449.
2 Zhou, Y. J., Kerkhoven, E. J. and Nielsen, J. (2018). Barriers and opportunities in bio-based production of hydrocarbons. Nature Energy 3, 925-935.
3 Blankenship, R. E., Tiede, D. M., Barber, J., Brudvig, G. W., Fleming, G., Ghirardi, M., Gunner, M. R., Junge, W., Kramer, D. M., Melis, A., Moore, T. A., Moser, C. C., Nocera, D. G., Nozik, A. J., Ort, D. R., Parson, W. W., Prince, R. C. and Sayre, R. T. (2011). Comparing Photosynthetic and Photovoltaic Efficiencies and Recognizing the Potential for Improvement. Science 332, 805-809.
4 Long, B., Fischer, B., Zeng, Y., Amerigian, Z., Li, Q., Bryant, H., Li, M., Dai, S. Y. and Yuan, J. S. (2022). Machine learning-informed and synthetic biology-enabled semi-continuous algal cultivation to unleash renewable fuel productivity. Nature Communications 13, 541.
5 Sun, Z., Ma, T., Tao, H., Fan, Q. and Han, B. (2017). Fundamentals and Challenges of Electrochemical CO₂ Reduction Using Two-Dimensional Materials. Chem 3, 560-587.
6 Zhang, Q., Yang, C., Guan, A., Kan, M. and Zheng, G. (2022). Photocatalytic CO₂ conversion: from C1 products to multi-carbon oxygenates. Nanoscale 14, 10268-10285.
7 Liu, M., Pang, Y., Zhang, B., De Luna, P., Voznyy, O., Xu, J., Zheng, X., Dinh, C. T., Fan, F., Cao, C., de Arquer, F. P. G., Safaei, T. S., Mepham, A., Klinkova, A., Kumacheva, E., Filleter, T., Sinton, D., Kelley, S. O. and Sargent, E. H. (2016). Enhanced electrocatalytic CO₂ reduction via field-induced reagent concentration. Nature 537, 382-386.
8 Klinkova, A., De Luna, P., Dinh, C.-T., Voznyy, O., Larin, E. M., Kumacheva, E. and Sargent, E. H. (2016). Rational Design of Efficient Palladium Catalysts for Electroreduction of Carbon Dioxide to Formate. ACS Catalysis 6, 8115-8120.
9 Zhang, G., Wang, T., Zhang, M., Li, L., Cheng, D., Zhen, S., Wang, Y., Qin, J., Zhao, Z.-J. and Gong, J. (2022). Selective CO₂ electroreduction to methanol via enhanced oxygen bonding. Nature Communications 13, 7768.
10 Li, F., Li, Y. C., Wang, Z., Li, J., Nam, D.-H., Lum, Y., Luo, M., Wang, X., Ozden, A., Hung, S.-F., Chen, B., Wang, Y., Wicks, J., Xu, Y., Li, Y., Gabardo, C. M., Dinh, C.-T., Wang, Y., Zhuang, T.-T., Sinton, D. and Sargent, E. H. (2020). Cooperative CO₂-to-ethanol conversion via enriched intermediates at molecule-metal catalyst interfaces. Nature Catalysis 3, 75-82.
11 Hann, E. C., Overa, S., Harland-Dunaway, M., Narvaez, A. F., Le, D. N., Orozco-Cárdenas, M. L., Jiao, F. and Jinkerson, R. E. (2022). A hybrid inorganic-biological artificial photosynthesis system for energy-efficient food production. Nature Food 3, 461-471.
12 Chen, X., Chen, J., Alghoraibi, N. M., Henckel, D. A., Zhang, R., Nwabara, U. O., Madsen, K. E., Kenis, P. J. A., Zimmerman, S. C. and Gewirth, A. A. (2021). Electrochemical CO₂-to-ethylene conversion on polyamine-incorporated Cu electrodes. Nature Catalysis 4, 20-27.
13 Choi, M., Bong, S., Kim, J. W. and Lee, J. (2021). Formation of 1-Butanol from CO₂ without *CO Dimerization on a Phosphorus-Rich Copper Cathode. ACS Energy Letters 6, 2090-2095.
14 Cronin, S. P., Dulovic, S., Lawrence, J. A., Filsinger, K. A., Hernandez-Gonzalez, A. P., Evans, R., Stiles, J. W., Morris, J., Pelczer, I. and Bocarsly, A. B. (2023). Direct Synthesis of 1-Butanol with High Faradaic Efficiency from CO₂ Utilizing Cascade Catalysis at a Ni-Enhanced (Cr203) 3Ga203 Electrocatalyst. Journal of the American Chemical Society 145, 6762-6772.
15 Zhang, P., Chen, K., Xu, B., Li, J., Hu, C., Yuan, J. S. and Dai, S. Y. (2022). Chem-Bio interface design for rapid conversion of CO₂ to bioplastics in an integrated system. Chem 8, 3363-3381.
16 Li, H., Opgenorth, P. H., Wernick, D. G., Rogers, S., Wu, T. Y., Higashide, W., Malati, P., Huo, Y. X., Cho, K. M. and Liao, J. C. (2012). Integrated electromicrobial conversion of CO₂ to higher alcohols. Science 335, 1596.
17 Xie, S., Sun, S., Lin, F., Li, M., Pu, Y., Cheng, Y., Xu, B., Liu, Z., da Costa Sousa, L., Dale, B. E., Ragauskas, A. J., Dai, S. Y. and Yuan, J. S. (2019). Mechanism-Guided Design of Highly Efficient Protein Secretion and Lipid Conversion for Biomanufacturing and Biorefining. Advance Science (Weinh) 6, 1801980.
18 Xiao, Y., Ruan, Z., Liu, Z., Wu, S. G., Varman, A. M., Liu, Y. and Tang, Y. J. (2013). Engineering Escherichia coli to convert acetic acid to free fatty acids. Biochemical Engineering Journal 76, 60-69.
19 Round, J. W., Roccor, R. and Eltis, L. D. (2019). A biocatalyst for sustainable wax ester production: re-wiring lipid accumulation in Rhodococcus to yield high-value oleochemicals. Green Chemistry 21, 6468-6482.
20 Round, J. A.-O., Robeck, L. A.-O. and Eltis, L. A.-O. (An Integrative Toolbox for Synthetic Biology in Rhodococcus. ACS Synthetic Biology 10, 2383-2395.
21 Liang, Y. and Yu, H. (2021). Genetic toolkits for engineering Rhodococcus species with versatile applications. Biotechnology Advances 49, 107748.
22 Henry, C. S., DeJongh, M., Best, A. A., Frybarger, P. M., Linsay, B. and Stevens, R. L. (2010). High-throughput generation, optimization and analysis of genome-scale metabolic models. Nature Biotechnology 28, 977-982.
23 Arkin, A. P., Cottingham, R. W., Henry, C. S., Harris, N. L., Stevens, R. L., Maslov, S., Dehal, P., Ware, D., Perez, F., Canon, S., Sneddon, M. W., Henderson, M. L., Riehl, W. J., Murphy-Olson, D., Chan, S. Y., Kamimura, R. T., Kumari, S., Drake, M. M., Brettin, T. S., Glass, E. M., Chivian, D., Gunter, D., Weston, D. J., Allen, B. H., Baumohl, J., Best, A. A., Bowen, B., Brenner, S. E., Bun, C. C., Chandonia, J.-M., Chia, J.-M., Colasanti, R., Conrad, N., Davis, J. J., Davison, B. H., DeJongh, M., Devoid, S., Dietrich, E., Dubchak, I., Edirisinghe, J. N., Fang, G., Faria, J. P., Frybarger, P. M., Gerlach, W., Gerstein, M., Greiner, A., Gurtowski, J., Haun, H. L., He, F., Jain, R., Joachimiak, M. P., Keegan, K. P., Kondo, S., Kumar, V., Land, M. L., Meyer, F., Mills, M., Novichkov, P. S., Oh, T., Olsen, G. J., Olson, R., Parrello, B., Pasternak, S., Pearson, E., Poon, S. S., Price, G. A., Ramakrishnan, S., Ranjan, P., Ronald, P. C., Schatz, M. C., Seaver, S. M. D., Shukla, M., Sutormin, R. A., Syed, M. H., Thomason, J., Tintle, N. L., Wang, D., Xia, F., Yoo, H., Yoo, S. and Yu, D. (2018). KBase: The United States Department of Energy Systems Biology Knowledgebase. Nature Biotechnology 36, 566-569.
24 Sun, S., Ding, Y., Liu, M., Xian, M. and Zhao, G. (2020). Comparison of Glucose, Acetate and Ethanol as Carbon Resource for Production of Poly (3-Hydroxybutyrate) and Other Acetyl-CoA Derivatives. Front Bioeng Biotechnol 8, 833.
25 Miao, Y., Liu, J., Wang, X., Liu, B., Liu, W. and Tao, Y. (2022). Fatty acid feedstocks enable a highly efficient glyoxylate-TCA cycle for high-yield production of β-alanine. mLife 1, 171-182.
26 Alvarez, H. M., Herrero, O. M., Silva, R. A., Hernandez, M. A., Lanfranconi, M. P. and Villalba, M. S. (2019). Insights into the Metabolism of Oleaginous Rhodococcus spp. Applied and Environmental Microbiology 85, e00498-00419.
27 Mori, M., Marinari, E. and De Martino, A. (2019). A yield-cost tradeoff governs Escherichia coli's decision between fermentation and respiration in carbon-limited growth. NPJ Systems Biology and Applications 5, 16.
28 De Angelis, M., Bini, L., Pallini, V., Cocconcelli, P. S. and Gobbetti, M. (2001). The acid-stress response in Lactobacillus sanfranciscensis CB1. Microbiology 147, 1863-1873.
29 Bearson, S., Bearson, B. and Foster, J. W. (1997). Acid stress responses in enterobacteria. FEMS Microbiology Letters 147, 173-180.
30 Wei, H., Shan, X., Wu, L., Zhang, J., Saleem, M., Yang, J., Liu, Z. and Chen, X. (2023). Microbial cell membrane properties and intracellular metabolism regulate individual level microbial responses to acid stress. Soil Biology and Biochemistry 177, 108883.
31 Lund, P., Tramonti, A. and De Biase, D. (2014). Coping with low pH: molecular strategies in neutralophilic bacteria. FEMS Microbiology Reviews 38, 1091-1125.
32 Halamka, T. A., Garcia, A., Evans, T. W., Schubert, S., Younkin, A., Hinrichs, K.-U. and Kopf, S. (2024). Occurrence of ceramides in the Acidobacterium Solibacter usitatus: implications for bacterial physiology and sphingolipids in soils. Frontiers in Geochemistry 2, 2024.
33 Yoshiyama, Y., Tanaka, K., Yoshiyama, K., Hibi, M., Ogawa, J. and Shima, J. (2015). Trehalose accumulation enhances tolerance of Saccharomyces cerevisiae to acetic acid. Journal of Bioscience and Bioengineering 119, 172-175.
34 Carvalho Ana, L., Cardoso Filipa, S., Bohn, A., Neves Ana, R. and Santos, H. (2011). Engineering Trehalose Synthesis in Lactococcus lactis for Improved Stress Tolerance. Applied and Environmental Microbiology 77, 4189-4199.
35 Schweizer, E. and Hofmann, J. (Microbial type I fatty acid synthases (FAS): major players in a network of cellular FAS systems. Microbiology and Molecular Biology Reviews 68, 501-517.
36 Long, C. P., Gonzalez, J. E., Feist, A. M., Palsson, B. O. and Antoniewicz, M. R. (2018). Dissecting the genetic and metabolic mechanisms of adaptation to the knockout of a major metabolic enzyme in Escherichia coli. Proceedings of the National Academy of Sciences 115, 222-227.
37 Shingler, V., Powlowski J Fau-Marklund, U. and Marklund, U. (Nucleotide sequence and functional analysis of the complete phenol/3,4-dimethylphenol catabolic pathway of Pseudomonas sp. strain CF600. Journal of Bacteriology 174, 711-724.
38 Papapetridis, I., van Dijk, M., Dobbe, A. P., Metz, B., Pronk, J. T. and van Maris, A. J. (2016). Improving ethanol yield in acetate-reducing Saccharomyces cerevisiae by cofactor engineering of 6-phosphogluconate dehydrogenase and deletion of ALD6. Microb Cell Fact 15, 67.
39 Chen, C., Yu, S., Yang, Y., Louisia, S., Roh, I., Jin, J., Chen, S., Chen, P.-C., Shan, Y. and Yang, P. (2022). Exploration of the bio-analogous asymmetric C-C coupling mechanism in tandem CO₂ electroreduction. Nature Catalysis 5, 878-887.
40 Zhou, Y., Che, F., Liu, M., Zou, C., Liang, Z., De Luna, P., Yuan, H., Li, J., Wang, Z., Xie, H., Li, H., Chen, P., Bladt, E., Quintero-Bermudez, R., Sham, T.-K., Bals, S., Hofkens, J., Sinton, D., Chen, G. and Sargent, E. H. (2018). Dopant-induced electron localization drives CO₂ reduction to C2 hydrocarbons. Nature Chemistry 10, 974-980.
41 Ren, D., Ang, B. S.-H. and Yeo, B. S. (2016). Tuning the Selectivity of Carbon Dioxide Electroreduction toward Ethanol on Oxide-Derived CuxZn Catalysts. ACS Catalysis 6, 8239-8247.
42 Wang, P., Yang, H., Tang, C., Wu, Y., Zheng, Y., Cheng, T., Davey, K., Huang, X. and Qiao, S.-Z. (2022). Boosting electrocatalytic CO₂-to-ethanol production via asymmetric C-C coupling. Nature Communications 13, 3754.
43 Li, Y., Shan, W., Zachman, M. J., Wang, M., Hwang, S., Tabassum, H., Yang, J., Yang, X., Karakalos, S., Feng, Z., Wang, G. and Wu, G. (2022). Atomically Dispersed Dual-Metal Site Catalysts for Enhanced CO₂ Reduction: Mechanistic Insight into Active Site Structures. Angewandte Chemie International Edition 61, e202205632.
44 Yang, Y., Ge, S., Pan, Y., Qian, W., Wang, S., Zhang, J. and Zhuang, L.-L. (2023). Screening of microalgae species and evaluation of algal-lipid stimulation strategies for biodiesel production. Science of The Total Environment 857, 159281.
45 Song, X., Liu, B.-F., Kong, F., Ren, N.-Q. and Ren, H.-Y. (2022). Overview on stress-induced strategies for enhanced microalgae lipid production: Application, mechanisms and challenges. Resources, Conservation and Recycling 183, 106355.
46 Singh, P., Kumari, S., Guldhe, A., Misra, R., Rawat, I. and Bux, F. (2016). Trends and novel strategies for enhancing lipid accumulation and quality in microalgae. Renewable and Sustainable Energy Reviews 55, 1-16.
47 Ahmad, L., Khordehgah, N., Malinauskaite, J. and Jouhara, H. (2020). Recent advances and applications of solar photovoltaics and thermal technologies. Energy 207, 118254.
48 Liu, C., Colón, B. C., Ziesack, M., Silver, P. A. and Nocera, D. G. (2016). Water splitting-biosynthetic system with CO₂ reduction efficiencies exceeding photosynthesis. Science 352, 1210-1213.
49 Zheng, T., Zhang, M., Wu, L., Guo, S., Liu, X., Zhao, J., Xue, W., Li, J., Liu, C., Li, X., Jiang, Q., Bao, J., Zeng, J., Yu, T. and Xia, C. (2022). Upcycling CO₂ into energy-rich long-chain compounds via electrochemical and metabolic engineering. Nature Catalysis 5, 388-396.
50 Bi, H., Wang, K., Xu, C., Wang, M., Chen, B., Fang, Y., Tan, X., Zeng, J. and Tan, T. (2023). Biofuel synthesis from carbon dioxide via a bio-electrocatalysis system. Chem Catalysis 3, 100557.
51 Parsons, S., Allen, M. J., Abeln, F., McManus, M. and Chuck, C. J. (2019). Sustainability and life cycle assessment (LCA) of macroalgae-derived single cell oils. Journal of Cleaner Production 232, 1272-1281.
52 Bonatsos, N., Marazioti, C., Moutousidi, E., Anagnostou, A., Koutinas, A. and Kookos, I. K. (2020). Techno-economic analysis and life cycle assessment of heterotrophic yeast-derived single cell oil production process. Fuel 264, 116839.
53 Shin, H., Hansen, K. U. and Jiao, F. (2021). Techno-economic assessment of low-temperature carbon dioxide electrolysis. Nature Sustainability 4, 911-919.
54 Davis, R., Aden, A. and Pienkos, P. T. (2011). Techno-economic analysis of autotrophic microalgae for fuel production. Applied Energy 88, 3524-3531.
55 Karamerou, E. E., Parsons, S., McManus, M. C. and Chuck, C. J. (2021). Using techno-economic modelling to determine the minimum cost possible for a microbial palm oil substitute. Biotechnol Biofuels 14, 57.
56 Seto, M., Kimbara, K., Shimura, M., Hatta, T., Fukuda, M. and Yano, K. (1995). A Novel Transformation of Polychlorinated Biphenyls by Rhodococcus sp. Strain RHA1. Applied and Environmental Microbiology 61, 3353-3358.
57 Trček, J., Mira, N. P. and Jarboe, L. R. (2015). Adaptation and tolerance of bacteria against acetic acid. Applied Microbiology and Biotechnology 99, 6215-6229.
58 Ingram, L. O. (1989). Ethanol Tolerance in Bacteria. Critical Reviews in Biotechnology 9, 305-319.
59 Allen, B. H., Gupta, N., Edirisinghe, J. N., Faria, J. P. and Henry, C. S. in Microbial Systems Biology: Methods and Protocols (ed Ali Navid) 291-320 (Springer US, 2022).
60 DeLorenzo, D. M., Rottinghaus, A. G., Henson, W. R. and Moon, T. S. (2018). Molecular Toolkit for Gene Expression Control and Genome Modification in Rhodococcus opacus PD630. ACS Synth Biol 7, 727-738.

We claim:

1. A composition for use in the production of diesel fuel lipid feedstock from CO₂, wherein the composition comprises an oleaginous microorganism, an electrocatalyst and a growth medium.

2. The composition of claim 1, wherein the oleaginous microorganism expresses or overexpresses one or more of the following enzymes: type 1 fatty acid synthase (FASI), diacylglycerol acyltransferase (DGAT), soluble pyridine nucleotide transhydrogenase and/or acetaldehyde dehydrogenase.

3. The composition of claim 1, wherein the oleaginous microorganism's genome comprises fasI and atf2.

4. The composition of claim 1, wherein the oleaginous microorganism's genome comprises sthA and dmpF.

5. The composition of claim 1, wherein the microorganism's genome comprises each of fasI, atf2, sthA and dmpF.

6. The composition of claim 1, wherein the microorganism is a Rhodococcus sp. selected from R. opacus and R. jostii.

7. The composition of claim 1, wherein the microorganism is Rhodoccocus jostii RHA1, and wherein the microorganism's genome is engineered to comprise each of fasI, atf2, sthA and dmpF.

8. The composition of claim 1, wherein the growth medium comprises ethanol and acetate as sole carbon sources.

9. The composition of claim 1, wherein the electrocatalyst has a formula expressed as CuₓZn_{y}, wherein X and Y are each any positive integer between 1-10.

10. The composition of claim 9, wherein the electrocatalyst is Cu₆Zn₁.

11. A method for producing a lipid biodiesel feedstock, the method comprising an electrocatalysis step, said electrocatalysis step comprising supplying electricity and CO₂ to an electrocatalyst to produce CO₂ intermediates via a reduction reaction between the CO₂ and the electrocatalyst; and
a bioconversion step, said bioconversion step comprising inoculating a growth medium with an oleaginous microorganism, supplying the growth medium with the CO₂ intermediates produced using the electrocatalyst, and allowing the microorganism to grow and synthesize lipids from the CO₂ intermediates into the growth medium, wherein oleaginous microorganism that expresses or overexpresses one or more of the following enzymes: type 1 fatty acid synthase (FASI), diacylglycerol acyltransferase (DGAT), soluble pyridine nucleotide transhydrogenase and acetaldehyde dehydrogenase.

12. The method of claim 11, wherein the electrocatalyst comprises a formula CuₓZn_{y}, wherein X and Y are each any positive integer between 1-10.

13. The method of claim 12, wherein the electrocatalysis operates at a current density of 100 to 125 mA/cm².

14. The method of claim 11, wherein CO₂ intermediates comprise ethanol, acetate, propanol and/or formate.

15. The method of claim 14, wherein the ethanol and acetate are generated at a ratio of 0.5 to 2.0, acetate/ethanol.

16. The method of claim 11, wherein the microorganism's genome comprises each of fasI, atf2, sthA and dmpF.

17. The method of claim 11, wherein the oleaginous microorganism is Rhodoccocus jostii RHA1, and wherein the microorganism's genome is engineered to comprise each of fasI, atf2, sthA and dmpF.

18. The method of claim 11, wherein the growth medium is inoculated with the microorganism at a cell density of 4 to 5 g/L.

19. The method of claim 11, further comprising extracting the lipids from the growth medium and generating a biodiesel from the lipids.

20. A system for continuous production of lipids from CO₂, the system comprising a two-chamber bioconversion unit and a CO₂ electrolysis unit containing an electrolyzer and an electrocatalyst.
